(19) United States
(12) Patent Application Publication
(10) Pub. No.: US 2023/0293509 A1
(43) Pub. Date: Sep. 21, 2023

(54) METHOD AND COMPOUND FOR USE, IN TREATING AND/OR PREVENTING NETOSIS

(71) Applicant: JUBILANT EPIPAD LLC, Bedminster, NJ (US)

(72) Inventors: Syed M I KAZMI, Bedminster, NJ (US); Sridharan RAJAGOPAL, Bangalore, Karnataka (IN); Dhanalakshmi SIVANANDAN, Bangalore, Karnataka (IN)

(21) Appl. No.: 18/041,393

(22) PCT Filed: Aug. 12, 2021
(86) PCT No.: PCT/IN2021/050776

§ 371 Date: Feb. 10, 2023

Related U.S. Application Data
(60) Provisional application No. 63/064,576, filed on Aug. 12, 2020.

(51) Int. Cl. A61K 31/4545; A61K 45/06; A61P 35/00; A61P 1/04; A61P 11/00; A61K 31/437; A61K 31/5383; A61K 31/497

(52) U.S. Cl. CPC A61K 31/4545; A61K 45/06; A61P 35/00; A61P 1/04; A61P 11/00; A61K 31/437; A61K 31/5383; A61K 31/497

(57) ABSTRACT

Described herein is a method for treating and/or preventing a disease or disorder or condition associated with NETosis, the method comprising: administering to a subject a compound as disclosed herein, or its polymorph, stereoisomer, prodrug, solvate, co-crystal, intermediate, pharmaceutically acceptable salt, metabolite, or composition thereof. The compound or its polymorph, stereoisomer, prodrug, solvate, co-crystal, intermediate, pharmaceutically acceptable salt, metabolite, or composition thereof may be used in for treating and/or preventing a disease or disorder or condition associated with NETosis.

This application claims the benefit of U.S. provisional patent application No. 63/064,576, filed on Aug. 12, 2020; the specifications of which are here incorporated by reference in their entirely.

### TECHNICAL FIELD

The present invention relates to methods of treating/preventing a disease or disorder or condition associated with NETosis. The present invention also related to compounds, its polymorph, stereoisomer, prodrug, solvate, co-crystal, intermediate, pharmaceutically acceptable salt, metabolite or composition thereof, for use in the treatment of a disease or disorder or condition ameliorated by inhibition of NETosis.

### BACKGROUND OF THE INVENTION

The protein arginine deiminase (PAD) consists of a family of enzymes that affect the process of citrullination in living tissues (J. E. Jones, et al., Curr. Opin. Drug Discov. Devel., 2009, 12, 616-627). The PAD family consists of PAD1, PAD2, PAD3, PAD4, and PAD6 individual enzymes. The enzymes of PAD family affect and regulate various physiological and pathological processes in human body and thus are important. The elevation in the levels of these enzymes has been implicated in various ailments, for example, cell differentiation (K. Nakashima et al., J. Biol. Chem., 1999, 274, 27786-27792), stem cell pluripotency (M. A. Christophorou et al., Nature, 2014, 507, 104-108), apoptosis (G. Y. Liu, Apoptosis, 2006, 11, 183-196), neutrophil extracellular trap (NET) formation (Y. Wang et al., J. Cell Biol., 2009, 184, 205-213), transcriptional regulation (P. Li et al., Mol. Cell Biol., 2008, 28, 4745-4758), antigen processing in autophagy (J. M. Ireland et al., J. Exp. Med., 2011, 208, 2625-2632), inflammation (D. Makrygiannakis et al., Ann. Rheum. Dis., 2006, 65, 1219-1222), the cornification of skin (E. Candi et al., Nat. Rev. Mol. Cell Biol., 2005, 6, 328-340), demyelination in multiple sclerosis (F. G. Mastronardi et al., J. Neurosci., 2006, 26, 11387-11396), chemokine regulation (T. Loos et al., Blood, 2008, 112, 2648-2656), spinal cord injury repair (S. Lange et al., Dev. Biol., 2011, 355, 205-214), and various normal cellular processes. Neutrophils are known to be recruited to the sites of infection where they kill various pathogens such as bacteria, fungi, and viruses by oxidative burst and phagocytosis (Schonrich et al., Front. Immunol., 2016, 7, 1-7). However, neutrophils also have another means of killing pathogens by the formation of NETs (Brinkmann et al., Science., 2004, 303 (5663), 1532-1535).

NETs are web like structures with DNA and proteins expelled from the neutrophils to capsulate pathogens. Although not completely understood, NET formation is well a regulated process. Peptidyl arginine deiminase type 4 (PAD4), which citrullinates histones to facilitate the decondensation and release of the chromosomal DNA plays an important role in this process of NET formation or NETosis. This process in fact forms a component of the innate immune system and the first line of defence against invading pathogens (Borregaard et al., Immunity, 2010, 33, 657-670).

NETs are composed of nuclear DNA that are decorated with a variety of nuclear and granular proteins, actively thrown out into the extracellular space, and result in death of the NET-producing cell (Brinkmann et al., Science., 2004, 303 (5663), 1532-1535). Cell death by this mechanism is unique from apoptosis and necrosis and has been termed “NETosis” (Zawrotniak et al., 2013, 60 (3), 277-284). Although NETs are beneficial in the host defence against pathogens, collateral damage from sustained NET formation also stimulates many disease processes, including those that occur during viral infections (Schonrich et al., Front. Immunol., 2016, 7, 1-7). Indeed, excessive NET formation can trigger a cascade of inflammatory reactions that promotes cancer cell metastasis, destroys surrounding tissues, facilitates microthrombosis, and results in permanent organ damage to the pulmonary, cardiovascular, and renal systems. Neutrophils have been shown to promote metastasis through several mechanisms, and predominantly through formation of NETs. Cytokines and chemokines secreted by tumors CXCL1 and GCSF are known to induce NET formation by neutrophils, which in turn leads to clustering of circulating tumor cells, enhances cell adhesion, and extravasation. In cancer cells without PAD4 expression, lung metastasis has been shown to be significantly delayed. PAD4 mediated citrullinated proteins have been shown to be a major driver of colon cancer liver metastasis. In colon cancer, PAD4 expression is much higher in liver metastasis compared to adjacent normal tissue or primary colon cancer and several ECM proteins have been shown to be highly citrullinated. In experimental models of liver metastasis, knock down or pharmacological inhibition of PAD4 lead to reduced citrullination and liver metastasis.

Neutrophil extracellular traps (NETs) are extracellular webs of chromatin, microbicidal proteins, and oxidant enzymes that are released by neutrophils to contain infections. However, NETs dysregulation in the formation or clearance will have the potential to propagate inflammation and microvascular thrombosis including in the lungs of patients with acute respiratory distress syndrome. Recently, several reports relate the role of NET's in ARDS and microvascular thrombosis from the sera of COVID-19 patients (Sigrun Lange, et. al., Int J Mol Sci., 2020, 21(13), 4662; Chiara Mozzini, et. al., Thromb Res., 2020, 191, 26-27; Betsy J. Barnes, et. al., J Exp Med., 2020, 217(6): e20200652; Carl Nathan., J Exp Med., 2020, 217(9), e20201439; Yu Zuo, et. al., JCI Insight., 2020, 5(11), e138999). Citrullinated histone 3 (Cit H3) and myeloperoxidase (MPO)-DNA are elevated in sera of COVID-19 patients which are specific markers of NET.

Recently several literatures report the involvement of NETs in various disease conditions such as RA, psoriasis, thrombosis, SLE, ARDS, Idiopathic pulmonary fibrosis, cystic fibrosis, etc. PAD4 is known for its involvement in the formation of neutrophil extracellular traps (NETs) and more specifically in the histone citrullination that occurs during NETosis (Cedervall., J et al., Oncoscience, 2015, 2(11), 900-901). Thus, PAD4 enzyme is linked to diseases characterized by abnormal levels of neutrophil extracellular traps (NETs).

Anti-neutrophil cytoplasmic antibody (ANCA) associated vasculitis (AAV) is an autoimmune disease characterized by inflammation of the small blood vessels. In vasculitis, there is loss of tolerance to enzyme myeloperoxidase (MPO) and proteinase (PR3) which lead to formation of ANCA to either MPO or PR3. In AAV, microscopic polyangiitis (MPA) is a subtype which is majorly driven by MPO intolerance and kidney is the major organ affected by MPA. In this MPO-ANCA vasculitis, NET formation is a key driver of pathogenesis and components of this pathway could be valuable prognostic markers as well as therapeutic targets for this disease.

Hidradenitis suppurativa (HS) is a painful and debilitating chronic inflammatory disease disorder of the pilosebaceous unit and apocrine gland-rich anatomical regions. It affects 1%-4% of the Western population, is characterized by recurrent painful abscess-like nodules with malodorous purulent drainage, sinus tracts, and scarring in the axillae, groin, anogenital region, and other intertriginous areas. Abundance of neutrophils has been reported in lesional skin of HS patients. HS neutrophils are primed to form NETs and that these structures associate with disease severity and progression. Further, HS sera contain autoantibodies targeting NET components and citrullinated proteins and increased circulating plasma cells. Therefore, putative role of NETs in immune dysregulation and pathophysiology of HS suggests them as potential new therapeutic targets for the treatment of this disease.

Therefore, ways of preventing or averting NETosis or neutralizing NETs are also of considerable interest.

PAD4 is predominantly expressed in granulocytes and is strongly linked to diverse diseases. In multiple tumors, PAD4 is found to be overexpressed affecting the p53 function and downstream pathways. Calcium binding to PAD4 promotes the bioactive conformation, increasing PAD4 activity by ten thousand times.

Slack et al. demonstrated the use of PAD4 inhibitors in the treatment of cancers (J. L. Slack et al., Cellular and Molecular Life Sciences, 2011, 68(4), 709-720). Overexpression of PAD4 had already been demonstrated in numerous cancers (X. Chang et al., BMC Cancer, 2009, 9, 40). It is suggested that PAD4 inhibitors have an anti-proliferative role as well. PAD4 deiminases arginine residues in histones at the promoters of p53-target genes such as p21, which are involved in cell cycle arrest and induction of apoptosis (P. Li et al., Molecular & Cell Biology, 2008, 28(15), 4745-4758).

PAD inhibition is a viable strategy for the treatment of numerous diseases mentioned above. The use of PAD inhibitors in various other diseases where dysregulated PAD activity is implicated needs to be explored. Although a definitive role for dysregulated PAD activity in these diseases has not been established, a direct link is plausible. However, there remains an unmet need to identify and develop PAD4 inhibitors which may treat PAD4 mediated disorders with efficacy. Specifically, for the prevention and treatment of liver cancer metastasis originating from colorectal cancer and pancreatic cancer, lung metastasis originating from breast cancer, and omentum metastasis originating from ovarian cancer. In particular, for the treatment of colorectal cancer patient with isolated liver metastasis who are candidate for surgery or other regional treatments to avoid the recurrence, therefore, disease-free survival.

### SUMMARY OF INVENTION

The present invention discloses agents which can bind specifically to the PAD4 protein and helps to reduce the comorbidities associated with viral infection specifically COVID-19 such as ARDS, increased pulmonary inflammation, and micro thrombosis.

In one aspect, the present disclosure provides a method for treating and/or preventing a disease or disorder or condition associated with NETosis, the method comprising: administering to a subject a compound selected from a group consisting of Formula (I), (R)-(3-aminopiperidin-1-yl)(2-(3-ethylbenzo[b]thiophen-2-yl)-3-methylimidazo[1,2-a]pyridin-7-yl)methanone (19), (R)-(3-aminopiperidin-1-yl)(2-(1-ethyl-5-phenyl-1H-pyrrol-2-yl)-3-methylimidazo[1,2-a]pyridin-7-yl)methanone (22), (R)-(3-aminopiperidin-1-yl)(2-(2-ethylphenyl)-3-methylimidazo[1,2-a]pyridin-7-yl)methanone (31), and (R)-(3-aminopiperidin-1-yl)(2-(3-ethylbenzo[b]thiophen-2-yl)-3,4-dihydro-5-oxa-1,2a-diazaacenaphthylen-7-yl)methanone (98), or its polymorph, stereoisomer, prodrug, solvate, co-crystal, intermediate, pharmaceutically acceptable salt, metabolite, or composition thereof;

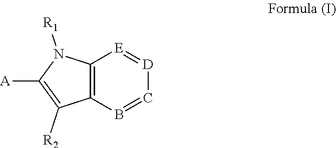

wherein,
A represents

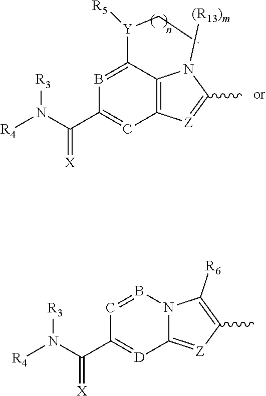

n is 0-2;
X is selected from 0 or S;
Y is selected from O, N, S, S(O), SO₂ or C;
Z is selected from N or CR₇;
B is selected from N or CR₈;
C is selected from N or CR₉;
D is selected from N or CR₁₀;
E is selected from N or CR₁₁;
R₁ is selected from a group consisting of hydrogen, C₁₋₆ alkyl, C₁₋₆ haloalkyl, C₁₋₆ alkoxy, C₃₋₆ cycloalkyl, C(O)C₁₋₆ alkyl, C(O)C₁₋₆ haloalkyl, C(O)NR₁₂, C(O)C₁₋₆ alkylamino, SO₂C₁₋₆ alkyl, SO₂C₁₋₆ haloalkyl, SO₂NR₁₂, SO₂NC₁₋₆ alkylamino, C₅₋₆ aryl, C₁₋₆ heteroaryl, C(O)C₅₋₆ aryl, C(O)C₁₋₆ heteroaryl, SO₂C₅₋₆ aryl, and SO₂C₁₋₆ heteroaryl;
wherein C₁₋₆ alkyl, C₁₋₆ alkoxy, C₃₋₆ cycloalkyl, C₅₋₆ aryl, C₁₋₆ heteroaryl, (CO)C₁₋₆ alkyl, C(O)C₁₋₆ haloalkyl, and SO₂C₁₋₆ alkyl, is optionally substituted with one or more groups selected from hydrogen, C₁₋₆ alkyl, C₁₋₆ alkoxy, C₃₋₈ cycloalkyl, C₅₋₆ aryl, C₁₋₆ heteroaryl, C₁₋₆ heterocyclyl, halogen, hydroxyl, —COOH, and cyano, and wherein C₁₋₆ alkyl, C₁₋₆ alkoxy, C₃₋₈ cycloalkyl, C₅₋₆ aryl, C₁₋₆ heteroaryl, C₁₋₆ heterocyclyl optionally further substituted one or more substituents selected from halogen, cyano, oxo, C₁₋₆ alkyl, C₁₋₆ alkoxy, and C₁₋₆ alkylhydroxy;
R₂, R₆, R₈, R₉, R₁₀, R₁₁, and R₁₃ are independently selected from hydrogen, hydroxyl, cyano, amino, halogen, C₁₋₆ alkyl, C₂₋₆ alkenyl, C₁₋₆ alkoxy, C₃₋₆ cycloalkyl, C₁₋₆ haloalkyl, C₁₋₆ haloalkoxy, C₁₋₆ acylamino, C₁₋₆ alkylamino, C₅₋₁₀ aryl, C₁₋₆ heterocyclyl, or C₁₋₆ heteroaryl;
wherein C₁₋₆ alkyl, C₂₋₆ alkenyl, C₁₋₆ alkoxy, C₁₋₆ heteroaryl, and C₁₋₆ haloalkoxy, is optionally substituted with one or more of the groups selected from hydrogen, oxo, C₁₋₆ alkyl, C₁₋₆ alkoxy, C₁₋₆ haloalkyl, C₃₋₆ cycloalkyl, C₅₋₁₀ aryl, C₁₋₆ heterocyclyl, C₁₋₆ heteroaryl, cyano, and hydroxyl;
m is 0 to 2;
R₃ is hydrogen;
R₄ is selected from C₁₋₆ alkylamino, and 5-10 membered monocyclic or bicyclic saturated heterocyclic ring with 1-3 heteroatoms selected from N, S or O; or
R₃ and R₄ can be taken together to form a 5-10 membered monocyclic or bicyclic saturated or unsaturated heterocyclic ring with 1-5 heteroatoms selected from N, S or 0;
wherein the 5-10 membered monocyclic or bicyclic saturated or unsaturated heterocyclic ring is optionally substituted with one or more substituents selected from a group consisting of amino, C₁₋₆ alkylamino, C₁₋₆ acylamino, —NHC(NH)CH₂Cl, —NH(CO)CH═CH—CH₂—N(CH₃)₂, C₁₋₆ alkyl, halogen, C₁₋₆ alkoxy, and hydroxyl;
R₅ is absent or is selected from a group consisting of hydrogen, C₁₋₆ alkyl, C₃₋₆ cycloalkyl, C₁₋₆ alkylamino, C₁₋₆ haloalkyl, C(O)C₁₋₆ alkyl, C(O)C₁₋₆ haloalkyl, C(O)NR₁₂, C(O)C₁₋₆ alkylamino, SO₂C₁₋₆ alkyl, SO₂C₁₋₆ haloalkyl, SO₂C₃₋₆ cycloalkyl, SO₂NR₁₂, SO₂NC₁₋₆ alkylamino, C₅₋₆ aryl, C₁₋₆ heteroaryl, C(O)C₅₋₆ aryl, C(O)C₁₋₆ heteroaryl, SO₂C₅₋₆ aryl, and SO₂C₁₋₆ heteroaryl;
wherein C₁₋₆ alkyl, (CO)C₁₋₆ alkyl, C(O)C₁₋₆ haloalkyl, SO₂C₅₋₆ aryl, and SO₂C₁₋₆ alkyl, is optionally substituted with C₁₋₆ alkoxy, halogen, C₅₋₆ aryl, and C₁₋₆ heteroaryl;
R₇ is selected from a group consisting of hydrogen, hydroxyl, cyano, halogen, C₁₋₆ alkyl, C₁₋₆ alkoxy, C₁₋₆ haloalkyl, C₁₋₆ haloalkoxy, C₁₋₆ acylamino, C₁₋₆ alkylamino, C₅₋₆ aryl, and C₁₋₆ heteroaryl;
wherein, C₁₋₆ alkoxy, and C₁₋₆ haloalkoxy, is optionally substituted with one or more of the groups selected from hydrogen, C₁₋₆ alkyl, C₁₋₆ alkoxy, C₁₋₆ haloalkyl, C₁₋₆ alkylhydroxy, cyano, and hydroxyl; and
R₁₂ is independently selected from hydrogen, and C₁₋₆ alkyl.

In another aspect of the present disclosure, there is provided a compound selected from a group consisting of Formula (I), (R)-(3-aminopiperidin-1-yl)(2-(3-ethylbenzo[b]thiophen-2-yl)-3-methylimidazo[1,2-a]pyridin-7-yl)methanone (19), (R)-(3-aminopiperidin-1-yl)(2-(1-ethyl-5-phenyl-1H-pyrrol-2-yl)-3-methylimidazo[1,2-a]pyridin-7-yl)methanone (22), (R)-(3-aminopiperidin-1-yl)(2-(2-ethylphenyl)-3-methylimidazo[1,2-a]pyridin-7-yl)methanone (31), and (R)-(3-aminopiperidin-1-yl)(2-(3-ethylbenzo[b]thiophen-2-yl)-3,4-dihydro-5-oxa-1,2a-diazaacenaphthylen-7-yl)methanone (98), or its polymorph, stereoisomer, prodrug, solvate, co-crystal, intermediate, pharmaceutically acceptable salt, metabolite or composition thereof, as described above, for use in the treatment of a disease or disorder or condition ameliorated by inhibition of NETosis.

These and other features, aspects, and advantages of the present subject matter will become better understood with reference to the following description. This summary is provided to introduce a selection of concepts in a simplified form. This summary is not intended to identify key features or essential features of the disclosure, nor is it intended to be used to limit the scope of the subject matter.

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions and compounds referred to or indicated in this specification, individually or collectively, and any and all combinations of any or more of such steps or features.

### Definitions

For convenience, before further description of the present disclosure, certain terms employed in the specification, and examples are collected here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The term “or” means “and/or” unless stated otherwise.

Throughout the description and the claims which follow, unless the context requires otherwise, the word “comprise”, and variations such as “comprises” and “comprising”, will be understood to imply the inclusion of a stated integer or step or group of integers but not to the exclusion of any other integer or step or group of integers or steps.

The term “including” as well as other forms, such as “include”, “includes’ and “included” is not limiting. For example, “including” used to mean “including but not limited to”. “Including” and “including but not limited to” are used interchangeably.

In the structural formulae given herein and throughout the present disclosure, the following terms have been indicated meaning, unless specifically stated otherwise.

The compounds as disclosed herein and its polymorphs, stereoisomers, prodrugs, solvates (including hydrates), complexes, co-crystals, radio-labelled derivatives, intermediates, pharmaceutically acceptable salts, and metabolites thereof can also be referred as “compound of the present disclosure”.

The compounds according to Formula (I) contain one or more asymmetric centres (also referred to as a chiral centres) and may, therefore, exist as individual enantiomers, diastereoisomers, epimers or other stereoisomeric forms such as d-isomers and i-isomers and mixtures thereof. Additionally, the compounds of the present invention may exist as geometric isomers. The present invention includes all cis, trans, syn, anti, entgegen (E) and zusammen (Z) isomers as well as the appropriate mixtures thereof. Chiral centres, such as chiral carbon atoms, may also be present in a substituent such as an alkyl group. Where the stereochemistry of a chiral centre present in Formula (I) or in any chemical structure illustrated herein, is not specified, the structure is intended to encompass any stereoisomer and all mixtures thereof. Thus, compounds according to Formula (I) and other compounds containing one or more chiral centres may be used as racemic modifications including racemic mixtures and racemates, enantiomerically-enriched mixtures, or as enantiomerically-pure individual stereoisomers.

Individual stereoisomers of a compound according to Formula (I) which contain one or more asymmetric centres may be resolved by methods known to those skilled in the art. For example, such resolution may be carried out (1) by formation of diastereoisomeric salts, complexes or other derivatives; (2) by selective reaction with a stereoisomer-specific reagent, for example by enzymatic oxidation or reduction; or (3) by gas-liquid or liquid chromatography in a chiral environment, for example, on a chiral support such as silica with a bound chiral ligand or in the presence of a chiral solvent. It will be appreciated that where the desired stereoisomer is converted into another chemical entity by one of the separation procedures described above, a further step is required to liberate the desired form.

Alternatively, specific stereoisomers may be synthesised by asymmetric synthesis using optically active reagents, substrates, catalysts or solvents, or by converting one enantiomer to the other by asymmetric transformation.

It is to be understood that the references herein to compounds of the present disclosure, and salts thereof covers the compounds as free bases, or as salts thereof, for example as pharmaceutically acceptable salts thereof. Thus, in one embodiment the invention is directed to a method for treating and/or preventing a disease or disorder or condition associated with NETosis, the method comprising: administering to a subject compounds of Formula (I) as the free base. In another embodiment, the invention is directed to a method for treating and/or preventing a disease or disorder or condition associated with NETosis, the method comprising: administering to a subject a compound as disclosed herein and salts thereof. In a further embodiment, the invention is directed to a method for treating and/or preventing a disease or disorder or condition associated with NETosis, the method comprising: administering to a subject compounds of the present disclosure and pharmaceutically acceptable salts thereof.

It will be appreciated that pharmaceutically acceptable salts of the compounds of the present disclosure may be prepared. Indeed, in certain embodiments of the invention, pharmaceutically acceptable salts of the compounds according to Formula (I) may be preferred over the respective free base because such salts impart greater stability or solubility to the molecule thereby facilitating formulation into a dosage form. Accordingly, the invention is further directed to compounds of Formula (I) and pharmaceutically acceptable salts thereof for use in the treatment of a disease or disorder or condition ameliorated by inhibition of NETosis.

“Enantiomeric excess” (ee) is the excess of one enantiomer over the other expressed as a percentage. In a racemic modification, since both enantiomers are present in equal amounts, the enantiomeric excess is zero (0% ee). However, if one enantiomer were enriched such that it constitutes 95% of the product, then the enantiomeric excess would be 90% ee (the amount of the enriched enantiomer, 95%, minus the amount of the other enantiomer, 5%).

“Enantiomerically enriched” refers to products whose enantiomeric excess (ee) is greater than zero. For example, ‘enantiomerically enriched’ refers to products whose enantiomeric excess is greater than 50% ee, greater than 75% ee, and greater than 90% ee. ‘Enantiomerically pure’ refers to products whose enantiomeric excess is 99% or greater.

The compounds of the invention may exist in solid or liquid form. In the solid state, the compounds of the invention may exist in crystalline or non-crystalline form, or as a mixture thereof. For compounds of the invention that are in crystalline form, the skilled artisan will appreciate that pharmaceutically acceptable solvates may be formed wherein solvent molecules are incorporated into the crystalline lattice during crystallization. Solvates may involve non-aqueous solvents such as ethanol, iso-propyl alcohol, N, N-dimethyl sulfoxide (DMSO), acetic acid, ethanolamine, and ethyl acetate, or they may involve water as the solvent that is incorporated into the crystalline lattice. Solvates wherein water is the solvent that is incorporated into the crystalline lattice are typically referred to as ‘hydrates’. Hydrates include stoichiometric hydrates as well as compositions containing variable amounts of water. The invention includes all such solvates.

It will be further appreciated that certain compounds of the invention that exist in crystalline form, including the various solvates thereof, may exhibit polymorphism (i.e. the capacity to occur in different crystalline structures). These different crystalline forms are typically known as ‘polymorphs’. The invention includes such polymorphs. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. It will be appreciated that different polymorphs may be produced, for example, by changing or adjusting the reaction conditions or reagents, used in making the compound. For example, changes in temperature, pressure, or solvent may result in polymorphs. In addition, one polymorph may spontaneously convert to another polymorph under certain conditions.

The invention also includes isotopically-labelled compounds, which are identical to the compounds of Formula (I) and salts thereof, but for the fact that one or more atoms are replaced by an atom having an atomic mass or mass number different from the atomic mass or mass number most commonly found in nature. Examples of isotopes that can be incorporated into the compounds of the invention include isotopes of hydrogen, carbon, nitrogen, oxygen and fluorine, such as ³H, ¹¹C, ¹⁴C and ⁸F.

The term “co-crystals” refers to solids that are crystalline single phase materials composed of two or more different molecular and/or ionic compounds generally in a stoichiometric ratio which are neither solvates nor simple salts.

As used herein, the term “substituted” or “optionally substituted” in reference to a group indicates that a hydrogen atom attached to a member atom within a group is replaced. It should be understood that the term ‘substituted’ includes the implicit provision that such substitution be in accordance with the permitted valence of the substituted atom and the substituent and that the substitution results in a stable compound (i.e. one that does not spontaneously undergo transformation such as rearrangement, cyclisation, or elimination). In certain embodiments, a single atom may be substituted with more than one substituent as long as such substitution is in accordance with the permitted valence of the atom.

As used herein, the term “substituted” or “optionally substituted” refers to replacement of one or more hydrogen radicals in a given structure with a radical of a specified substituent including, but not limited to: halo, alkyl, alkenyl, alkynyl, aryl, heterocyclyl, thiol, alkylthio, arylthio, alkylthioalkyl, arylthioalkyl, alkylsulfonyl, alkylsulfonylalkyl, arylsulfonylalkyl, alkoxy, aryloxy, aralkoxy, aminocarbonyl, alkylaminocarbonyl, arylaminocarbonyl, alkoxycarbonyl, aryloxycarbonyl, haloalkyl, amino, trifluoromethyl, cyano, nitro, alkylamino, arylamino, alkylaminoalkyl, arylaminoalkyl, aminoalkylamino, hydroxy, alkoxyalkyl, carboxyalkyl, alkoxycarbonylalkyl, aminocarbonylalkyl, acyl, aralkoxycarbonyl, carboxylic acid, sulfonic acid, sulfonyl, phosphonic acid, cycloalkyl, heteroaryl, and aliphatic. It is understood that the substituent may be further substituted.

As used herein, the term “polymorphs” refers to crystal forms of the same molecule, and different polymorphs may have different physical properties such as, for example, melting temperatures, heats of fusion, solubilities, dissolution rates and/or vibrational spectra as a result of the arrangement or conformation of the molecules in the crystal lattice.

As used herein, the term “prodrugs” refers to the precursor of the compound of Formula (I) which on administration undergoes chemical conversion by metabolic processes before becoming active pharmacological substances. In general, such prodrugs will be functional derivatives of a compound of the invention, which are readily convertible in vivo into a compound of the invention.

As used herein, unless otherwise defined the term “alkyl” alone or in combination with other term(s) means saturated aliphatic hydrocarbon chain having the specified number of carbon atoms. For example, C₁-C₁₀ straight or C₁-C₁₀ branched alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Representative branched alkyl groups have one, two, or three branches. Examples of “alkyl” include but are not limited to methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “alkenyl” refers to aliphatic hydrocarbon chain having the specified number of carbon atoms with one or more double bond in the hydrocarbon chain between carbon atoms. C₂₋₆ alkenyl in the present disclosure refers to the hydrocarbon with 2 to 6 carbon atoms with one, two or more double bond. The alkenyl group may be linear or branched and may be optionally substituted.

As used herein, the term “cyano” refers to a —CN group.

As used herein, the term “oxo” refers to a ═O group.

As used herein, the term “amino” refers to a —NH₂ group.

As used herein, the term “hydroxy” or “hydroxyl” refers to a —OH group.

As used herein, the term “C(O) alkyl” refers to an alkyl group as defined above attached via carbonyl linkage to the rest of the molecule. For example, C(O)C₁₋₆ alkyl refers to an alkyl group having from 1-6 carbon atoms, or 1-3 carbon atoms attached via carbonyl linkage to the rest of the molecule. Preferred C(O) alkyl groups include, without limitation, —C(O)CH₃¬, —C(O)CH₂CH₃, and the like.

The term “SO₂ alkyl” refers to an alkyl group as defined above attached via sulfonyl linkage to the rest of the molecule. For example, SO₂C₁₋₆ alkyl refers to an alkyl group having from 1-6 carbon atoms, or 1-3 carbon atoms attached via sulfonyl linkage to the rest of the molecule. Preferred SO₂ alkyl groups include, without limitation, —SO₂CH₃¬, —SO₂CH₂CH₃, and the like.

The term “alkoxy” refers to an alkyl group attached via an oxygen linkage to the rest of the molecule. For example, C₁₋₆ alkoxy refers to an alkyl group having from 1-6 carbon atoms, or 1-3 carbon atoms attached via an oxygen linkage to the rest of the molecule. Preferred alkoxy groups include, without limitation, —OCH₃ (methoxy), —OC₂H₅(ethoxy) and the like.

The term “alkylamino” refers to an alkyl group as defined above attached via amino linkage to the rest of the molecule. For example, C₁₋₆ alkylamino refers to an alkyl group having from 1-6 carbon atoms, or 1-3 carbon atoms attached via amino linkage to the rest of the molecule. Preferred alkylamino groups include, without limitation, —NHCH₃¬, —N(CH₃)₂, and the like.

The term “alkylhydroxy” refers to an alkyl group as defined above attached hydroxyl group. For example, C₁₋₆ alkylhydroxy refers to an alkyl group having from 1-6 carbon atoms, or 1-3 carbon atoms attached to hydroxyl group. Preferred alkylhydroxy groups include, without limitation, —CH₂OH, —CH₂—CH₂—OH, —(CH₃)₂—CH₂—OH, and the like.

The term “C(O)NR” refers to an alkylamino group as defined above attached via a carbonyl linkage to the rest of the molecule. Preferred C(O)NR groups include, C(O)NCH₃, C(O)NCH₂CH₃, and the like.

The term “SO₂NR” refers to an alkylamino group as defined above attached via a sulfonyl linkage to the rest of the molecule. Preferred SO₂NR groups include, SO₂NCH₃, SO₂NCH₂CH₃, and the like.

The term “C(O) alkylamino” refers to an alkylamino group as defined above attached via carbonyl linkage to the rest of the molecule. For example, C(O)C₁₋₆ alkylamino refers to an alkylamino group having from 1-6 carbon atoms, or 1-3 carbon atoms attached via carbonyl linkage to the rest of the molecule. Preferred C(O) alkylamino groups include, without limitation, —C(O)NHCH₃¬, —C(O)N(CH₃)₂, and the like.

The term “SO₂ alkylamino” refers to an alkylamino group as defined above attached via sulfonyl linkage to the rest of the molecule. For example, SO₂C₁₋₆ alkylamino refers to an alkylamino group having from 1-6 carbon atoms, or 1-3 carbon atoms attached via sulfonyl linkage to the rest of the molecule. Preferred S02 alkylamino groups include, without limitation, —SO₂NHCH₃¬, —SO₂N(CH₃)₂, and the like.

The term “acylamino” refers to an acyl group attached via amino linkage to the rest of the molecule. For example, C₁₋₆ acylamino refers to an acyl group having from 1-6 carbon atoms, or 1-3 carbon atoms attached via amino linkage to the rest of the molecule. Preferred acylamino groups include, without limitation, —(CO)NHCH₃¬, —(CO)N(CH₃)₂, and the like.

The term “halo” or “halogen” refers to a halogen radical, alone or in combination with other term(s), for example, fluorine, chlorine, bromine, or iodine.

The term “haloalkyl” refers to an alkyl group as defined above attached via halo linkage to the rest of the molecule. For example, C₁₋₆ haloalkyl refers to an alkyl group having from 1-6 carbon atoms, or 1-3 carbon atoms attached via halo linkage to the rest of the molecule. Preferred haloalkyl groups include, without limitation, —CH₂¬Cl¬, —CHCl₂, and the like.

The term “C(O) haloalkyl” refers to an haloalkyl group as defined above attached via carbonyl linkage to the rest of the molecule. For example, C(O)C₁₋₆ haloalkyl refers to an haloalkyl group having from 1-6 carbon atoms, or 1-3 carbon atoms attached via carbonyl linkage to the rest of the molecule. Preferred C(O) haloalkyl groups include, without limitation, —C(O)CH₂Cl, —C(O)CHCl₂, and the like.

The term “SO₂ haloalkyl” refers to an haloalkyl group as defined above attached via sulfonyl linkage to the rest of the molecule. For example, SO₂C₁₋₆ haloalkyl refers to an haloalkyl group having from 1-6 carbon atoms, or 1-3 carbon atoms attached via sulfonyl linkage to the rest of the molecule. Preferred SO₂ haloalkyl groups include, without limitation, —SO₂CH₂Cl, —SO₂CHCl₂, and the like.

The term “haloalkoxy” refers to an alkoxy group as defined above attached via halo linkage to the rest of the molecule. For example, C₁₋₆ haloalkoxy refers to an alkoxy group having from 1-6 carbon atoms, or 1-3 carbon atoms attached via halo linkage to the rest of the molecule. Preferred haloalkoxy groups include, without limitation, —OCH₂Cl, —OCHCl₂, and the like.

The term “cycloalkyl” refers to a saturated hydrocarbon ring having a specified number of carbon atoms. For example, which are not limited, C₃₋₈ cycloalkyl refers to a cycloalkyl group having from 3 to 8 member atoms, or 3 member atoms. Preferred cycloalkyl groups include, without limitation, cyclopropyl, cyclobutyl, cyclopentyl, cyclohexyl, cycloheptyl, cyclooctyl, groups and the like.

The term “SO₂C₃₋₆ cycloalkyl” refers to an cycloalkyl group as defined above attached via sulfonyl linkage to the rest of the molecule. For example, SO₂C₃₋₆ cycloalkyl refers to an cycloalkyl group having from 3-6 carbon atoms attached via sulfonyl linkage to the rest of the molecule. Preferred SO₂ cycloalkyl groups include, without limitation, —SO₂C₃ cycloalkyl, and the like.

The term “aryl” refers to aromatic ring having a specified number of carbon atoms. For example, C₅₋₆ aryl refers to an aryl group having 5 or 6 member atoms, or 6 member atoms. Preferred aryl groups include, without limitation, phenyl, and the like.

The term “C(O) aryl” refers to an aryl group as defined above attached via carbonyl linkage to the rest of the molecule. For example, C(O)C₅₋₆ aryl refers to an alkyl group having from 5-6 carbon atoms attached via carbonyl linkage to the rest of the molecule. Preferred C(O) aryl groups include, without limitation, —C(O) C₆H₅¬, —C(O) C₅H₅, and the like.

The term “SO₂ aryl” refers to an aryl group as defined above attached via sulfonyl linkage to the rest of the molecule. For example, SO₂C₅₋₆ aryl refers to an aryl group having from 5-6 carbon atoms attached via sulfonyl linkage to the rest of the molecule. Preferred SO₂ aryl groups include, without limitation, —SO₂C₆H₅¬, —SO₂C₅H₅, and the like.

As used herein, the term “heteroaryl” refers to an aromatic heterocyclic ring system containing 5 to 20 ring atoms, suitably 5 to 10 ring atoms, which may be a single ring (monocyclic) or multiple rings (bicyclic, tricyclic or polycyclic) fused together or linked covalently. Preferably, “heteroaryl” is a 5- to 6-membered ring. Further, the “C₁₋₆ heteroaryl” rings having 1 or 6 carbon as member atoms. The “heteroaryl” includes pyridinyl, tetrazolyl and pyrazolyl. The rings may contain from 1 to 4 heteroatoms selected from N, O and S, wherein the N or S atom is optionally oxidized or the N atom is optionally quarternized. Any suitable ring position of the heteroaryl moiety may be covalently linked to the defined chemical structure. Examples of “heteroaryl” include but are not limited to furanyl, thienyl, pyrrolyl, pyrazolyl, imidazolyl, oxazolyl, cinnolinyl, isoxazolyl, thiazolyl, isothiazolyl, 1H-tetrazolyl, oxadiazolyl, triazolyl, pyridyl, 3-fluoropyridyl, pyrimidinyl, pyrazinyl, pyridazinyl, benzoxazolyl, benzisoxazolyl; benzothiazolyl, benzofuranyl, benzothienyl, benzotriazinyl, phthalazinyl, thianthrene, dibenzofuranyl, dibenzothienyl, benzimidazolyl, indolyl, isoindolyl, indazolyl, quinolinyl, isoquinolinyl, quinazolinyl, quinoxalinyl, purinyl, pteridinyl, 9H-carbazolyl, α-carbolinyl, indolizinyl, benzoisothiazolyl, benzoxazolyl, pyrrolopyridyl, furopyridinyl, purinyl, benzothiadiazolyl, benzooxadiazolyl, benzotriazolyl, benzotriadiazolyl, carbazolyl, dibenzothienyl, acridinyl and the like. Heteroaryl group may be optionally further substituted.

The term “heteroatom” as used herein designates a sulfur, nitrogen or oxygen atom.

The term “C(O) heteroaryl” refers to an heteroaryl group as defined above attached via carbonyl linkage to the rest of the molecule. For example, C(O)C₁₋₆ heteroaryl refers to an alkyl group having from 1-6 carbon atoms attached via carbonyl linkage to the rest of the molecule. Preferred C(O) heteroaryl groups include, without limitation, —C(O) pyridinyl, —C(O) pyrazolyl, and the like.

The term “SO₂ heteroaryl” refers to an aryl group as defined above attached via sulfonyl linkage to the rest of the molecule. For example, SO₂C₁₋₆ heteroaryl refers to an aryl group having from 1-6 carbon atoms attached via sulfonyl linkage to the rest of the molecule. Preferred SO₂ heteroaryl groups include, without limitation, —SO₂ pyridinyl, —SO₂ pyrazolyl, and the like.

The term “heterocyclic” and “heterocyclyl” refer to saturated or unsaturated monocyclic aliphatic rings containing 1 to 6 carbon atoms including 1 to 5 heteroatoms which may be saturated or unsaturated bicyclic aliphatic rings containing 5, 6 or 7 ring members including 1 or 2 heteroatoms. In certain embodiments, ‘heterocyclyl’ groups are saturated. In other embodiments, ‘heterocyclyl’ groups are unsaturated. ‘Heterocyclyl’ groups containing more than one heteroatom may contain different heteroatoms. ‘Heterocyclyl’ groups may be substituted with one or more substituents as defined herein. ‘Heterocyclyl’ includes piperidinyl, tetrahydropyranyl, azepinyl, oxazepinyl, azabicyclo[3.1.0]hexanyl.

The phrase “pharmaceutically acceptable” refers to those compounds, materials, compositions, and dosage forms which are, within the scope of sound medical judgment, suitable for use in contact with the tissues of human beings and animals without excessive toxicity, irritation, or other problem or complication, commensurate with a reasonable benefit/risk ratio.

As used herein, the term “pharmaceutically acceptable salts” refers to salts that retain the desired biological activity of the subject compound and exhibit minimal undesired toxicological effects. These pharmaceutically acceptable salts may be prepared in situ during the final isolation and purification of the compound, or by separately reacting the purified compound in its free base form with a suitable acid.

As used herein, the term “composition” is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

As used herein, the term “pharmaceutical composition” refers to a composition(s) containing a therapeutically effective amount of at least one compound of the present disclosure or its pharmaceutically acceptable salt; and a conventional pharmaceutically acceptable carrier.

The pharmaceutical composition(s) of the present invention can be administered orally, for example in the form of tablets, coated tablets, pills, capsules, granules or elixirs. Administration, however, can also be carried out rectally, for example in the form of suppositories, or parenterally, for example intravenously, intramuscularly or subcutaneously, in the form of injectable sterile solutions or suspensions, or topically, for example in the form of ointments or creams or transdermals, in the form of patches, or in other ways, for example in the form of aerosols or nasal sprays.

As used here in “pharmaceutical composition” refers to a composition which may be a PAD4 inhibitor and can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain an effective amount of the pharmaceutically acceptable salt of the compounds of the present disclosure, and may be prepared by methods of pharmacy well known to those skilled in the art.

The pharmaceutical composition(s) usually contain(s) 1% to 99%, for example, 5% to 75%, or from 10% to 30% by weight of the compound of formula (I) or pharmaceutically acceptable salts thereof. The amount of the compound of formula (I) or pharmaceutically acceptable salts thereof in the pharmaceutical composition(s) can range from 1 mg to 1000 mg or from 2.5 mg to about 500 mg or from 5 mg to 250 mg or in any range falling within the broader range of 1 mg to 1000 mg or higher or lower than the afore mentioned range.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder or condition. The term “treating” includes reducing or alleviating at least one adverse effect or symptom of a condition, disease or disorder or condition described herein. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “therapeutically effective amount” refers to that amount of a compound or its polymorph, stereoisomer, prodrug, solvate, co-crystal, intermediate, pharmaceutically acceptable salt, metabolite, or composition thereof; or a composition comprising the compound or its polymorph, stereoisomer, prodrug, solvate, co-crystal, intermediate, pharmaceutically acceptable salt or metabolite thereof, effective in producing the desired therapeutic response in a particular patient suffering from a diseases or disorder, in particular their use in diseases or disorder associated with cancer. Particularly, the term “therapeutically effective amount” includes the amount of the compound or its polymorph, stereoisomer, prodrug, solvate, co-crystal, intermediate, pharmaceutically acceptable salt, metabolite, or composition thereof, when administered, that induces a positive modification in the disease or disorder or condition to be treated or is sufficient to prevent development of, or alleviate to some extent, one or more of the symptoms of the disease or disorder or condition being treated in a subject. In respect of the therapeutic amount of the compound, the amount of the compound used for the treatment of a subject is low enough to avoid undue or severe side effects, within the scope of sound medical judgment can also be considered. The therapeutically effective amount of the compound or composition will be varied with the particular condition being treated, the severity of the condition being treated or prevented, the duration of the treatment, the nature of concurrent therapy, the age and physical condition of the end-user, the specific compound or composition employed the particular pharmaceutically acceptable carrier utilized.

Salts and solvates having non-pharmaceutically acceptable counter-ions or associated solvents are within the scope of the present invention, for example, for use as intermediates in the preparation of other compounds of of the present disclosure and its pharmaceutically acceptable salts. Thus, one embodiment of the invention embraces compounds of Formula (I) and salts thereof. Compounds according to Formula (I) contain a basic functional group and are therefore capable of forming pharmaceutically acceptable acid addition salts by treatment with a suitable acid. Suitable acids include pharmaceutically acceptable inorganic acids and pharmaceutically acceptable organic acids. Representative pharmaceutically acceptable acid addition salts include hydrochloride, hydrobromide, nitrate, methylnitrate, sulfate, bisulfate, sulfamate, phosphate, acetate, hydroxyacetate, phenyl acetate, propionate, butyrate, iso-butyrate, valerate, maleate, hydroxymaleate, acrylate, fumarate, malate, tartrate, citrate, salicylate, glycollate, lactate, heptanoate, phthalate, oxalate, succinate, benzoate, o-acetoxybenzoate, chlorobenzoate, methylbenzoate, dinitrobenzoate, hydroxybenzoate, methoxybenzoate, naphthoate, hydroxynaphthoate, mandelate, tannate, formate, stearate, ascorbate, palmitate, oleate, pyruvate, pamoate, malonate, laurate, glutarate, glutamate, estolate, methanesulfonate (mesylate), ethanesulfonate (esylate), 2-hydroxyethanesulfonate, benzenesulfonate (besylate), aminobenzenesulfonate, p-toluenesulfonate (tosylate), and naphthalene-2-sulfonate.

The term “PAD inhibitor” or “inhibitor of PAD” is used to identify a compound, which is capable of interacting with neutrophil extracellular traps (NETs) and more specifically in the histone citrullination that occurs during NETosis. Inhibiting PAD4 enzymatic activity means reducing the ability of PAD4 enzyme to inhibit the formation of citrulline through citrullination process. Preferably, such inhibition is specific to PAD enzyme.

The term “NET” or “neutrophil extracellular trap” refers to an extracellular complex of nucleosomes and proteins, e.g. proteins having antimicrobial activity. Upon activation, neutrophils and other cells undergo a cell death program termed “NETosis” and release portions of nuclear DNA in the form of nucleosomes in complex with various proteins having antimicrobial activity (i.e. NETs). Release of NETs from neutrophils has been associated with inflammation and microthrombosis during sepsis and non-infectious diseases and demonstrated to contribute to the pathology of various diseases described herein. NETosis refers to the release of NETs without concomitant cell death of the neutrophil.

A term once described, the same meaning applies for it, throughout the disclosure.

Treatment and/or prevention of NETosis by protein arginine deiminase (PAD) inhibition significantly reduced mortality, tissue damage, and inflammation therefore PAD4 inhibitors may be used for reducing the comorbidities associated with viral infection specifically COVID-19 (corona virus disease of 2019) such as ARDS, anti neutrophil cytoplasmic antibody (ANCA) associated vasculitis (AAV), hidradenitis suppurativa (HS), increased pulmonary inflammation, and micro thrombosis.

In an embodiment of the present disclosure, there is provided a method for treating and/or preventing a disease or disorder or condition associated with NETosis, the method comprising: administering to a subject a compound selected from a group consisting of Formula (I), (R)-(3-aminopiperidin-1-yl)(2-(3-ethylbenzo[b]thiophen-2-yl)-3-methylimidazo[1,2-a]pyridin-7-yl)methanone (19), (R)-(3-aminopiperidin-1-yl)(2-(1-ethyl-5-phenyl-1H-pyrrol-2-yl)-3-methylimidazo[1,2-a]pyridin-7-yl)methanone (22), (R)-(3-aminopiperidin-1-yl)(2-(2-ethylphenyl)-3-methylimidazo[1,2-a]pyridin-7-yl)methanone (31), and (R)-(3-aminopiperidin-1-yl)(2-(3-ethylbenzo[b]thiophen-2-yl)-3,4-dihydro-5-oxa-1,2a-diazaacenaphthylen-7-yl)methanone (98), or its polymorph, stereoisomer, prodrug, solvate, co-crystal, intermediate, pharmaceutically acceptable salt, metabolite or composition thereof;

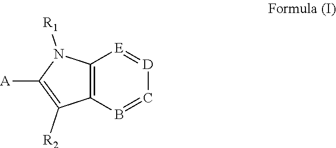

wherein,
A represents;

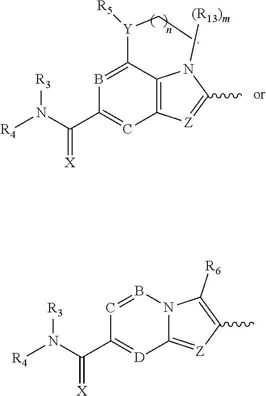

n is 0-2;
X is selected from O or S;
Y is selected from O, N, S, S(O), SO₂ or C;
Z is selected from N or CR₇;
B is selected from N or CR₈;
C is selected from N or CR₉;
D is selected from N or CR₁₀;
E is selected from N or CR₁₁;
R₁ is selected from a group consisting of hydrogen, C₁₋₆ alkyl, C₁₋₆ haloalkyl, C₁₋₆ alkoxy, C₃₋₆ cycloalkyl, C(O)C₁₋₆ alkyl, C(O)C₁₋₆ haloalkyl, C(O)NR₁₂, C(O)C₁₋₆ alkylamino, SO₂C₁₋₆ alkyl, SO₂C₁₋₆ haloalkyl, SO₂NR₁₂, SO₂NC₁₋₆ alkylamino, C₅₋₆ aryl, C₁₋₆ heteroaryl, C(O)C₅₋₆ aryl, C(O)C₁₋₆ heteroaryl, SO₂C₅₋₆ aryl, and SO₂C₁₋₆ heteroaryl; wherein, C₁₋₆ alkyl, C₁₋₆ alkoxy, C₃₋₆ cycloalkyl, C₅₋₆ aryl, C₁₋₆ heteroaryl, (CO)C₁₋₆ alkyl, C(O)C₁₋₆ haloalkyl, and SO₂C₁₋₆ alkyl, is optionally substituted with one or more groups selected from hydrogen, C₁₋₆ alkyl, C₁₋₆ alkoxy, C₃₋₈ cycloalkyl, C₅₋₆ aryl, C₁₋₆ heteroaryl, C₁₋₆ heterocyclyl, halogen, hydroxyl, —COOH, and cyano; wherein C₁₋₆ alkyl, C₁₋₆ alkoxy, C₃₋₈ cycloalkyl, C₅₋₆ aryl, C₁₋₆ heteroaryl, C₁₋₆ heterocyclyl optionally further substituted one or more substituents selected from halogen, cyano, oxo, C₁₋₆ alkyl, C₁₋₆ alkoxy, and C₁₋₆ alkylhydroxy;
R₂, R₆, R₈, R₉, R₁₀, R₁₁, and R₁₃ are independently selected from hydrogen, hydroxyl, cyano, amino, halogen, C₁₋₆ alkyl, C₂₋₆ alkenyl, C₁₋₆ alkoxy, C₃₋₆ cycloalkyl, C₁₋₆ haloalkyl, C₁₋₆ haloalkoxy, C₁₋₆ acylamino, C₁₋₆ alkylamino, C₅₋₁₀ aryl, C₁₋₆ heterocyclyl, or C₁₋₆ heteroaryl; wherein, C₁₋₆ alkyl, C₂₋₆ alkenyl, C₁₋₆ alkoxy, C₁₋₆ heteroaryl, and C₁₋₆ haloalkoxy, is optionally substituted with one or more of the groups selected from hydrogen, oxo, C₁₋₆ alkyl, C₁₋₆ alkoxy, C₁₋₆ haloalkyl, C₃₋₆ cycloalkyl, C₅₋₁₀ aryl, C₁₋₆ heterocyclyl, C₁₋₆ heteroaryl, cyano, and hydroxyl;
m is 0 to 2;
R₃ is hydrogen;
R₄ is selected from C₁₋₆ alkylamino, and 5-10 membered monocyclic or bicyclic saturated heterocyclic ring with 1-3 heteroatoms selected from N, S or O, or R₃ and R₄ can be taken together to form a 5-10 membered monocyclic or bicyclic saturated or unsaturated heterocyclic ring with 1-5 heteroatoms selected from N, S or O; wherein, the 5-10 membered monocyclic or bicyclic saturated or unsaturated heterocyclic ring is optionally substituted with one or more substituents selected from a group consisting of amino, C₁₋₆ alkylamino, C₁₋₆ acylamino, —NHC(NH)CH₂Cl, —NH(CO)CH═CH—CH₂—N(CH₃)₂, C₁₋₆ alkyl, halogen, C₁₋₆ alkoxy, and hydroxyl;
R₅ is absent or is selected from a group consisting of hydrogen, C₁₋₆ alkyl, C₃₋₆ cycloalkyl, C₁₋₆ alkylamino, C₁₋₆ haloalkyl, C(O)C₁₋₆ alkyl, C(O)C₁₋₆ haloalkyl, C(O)NR₁₂, C(O)C₁₋₆ alkylamino, SO₂C₁₋₆ alkyl, SO₂C₁₋₆ haloalkyl, SO₂C₃₋₆ cycloalkyl, SO₂NR₁₂, SO₂NC₁₋₆ alkylamino, C₅₋₆ aryl, C₁₋₆ heteroaryl, C(O)C₅₋₆ aryl, C(O)C₁₋₆ heteroaryl, SO₂C₅₋₆ aryl, and SO₂C₁₋₆ heteroaryl; wherein, C₁₋₆ alkyl, (CO)C₁₋₆ alkyl, C(O)C₁₋₆ haloalkyl, SO₂C₅₋₆ aryl, and SO₂C₁₋₆ alkyl, is optionally substituted with C₁₋₆ alkoxy, halogen, C₅₋₆ aryl, and C₁₋₆ heteroaryl;
R₇ is selected from a group consisting of hydrogen, hydroxyl, cyano, halogen, C₁₋₆ alkyl, C₁₋₆ alkoxy, C₁₋₆ haloalkyl, C₁₋₆ haloalkoxy, C₁₋₆ acylamino, C₁₋₆ alkylamino, C₅₋₆ aryl, and C₁₋₆ heteroaryl; wherein, C₁₋₆ alkoxy, and C₁₋₆ haloalkoxy, is optionally substituted with one or more of the groups selected from hydrogen, C₁₋₆ alkyl, C₁₋₆ alkoxy, C₁₋₆ haloalkyl, C₁₋₆ alkylhydroxy, cyano, and hydroxyl; and
R₁₂ is independently selected from hydrogen, and C₁₋₆ alkyl.

In an embodiment of the present disclosure, there is provided a method for treating and/or preventing a disease or disorder or condition associated with NETosis, the method comprising: administering to a subject a compound selected from a group consisting of Formula (I), (R)-(3-aminopiperidin-1-yl)(2-(3-ethylbenzo[b]thiophen-2-yl)-3-methylimidazo[1,2-a]pyridin-7-yl)methanone (19), (R)-(3-aminopiperidin-1-yl)(2-(1-ethyl-5-phenyl-1H-pyrrol-2-yl)-3-methylimidazo[1,2-a]pyridin-7-yl)methanone (22), (R)-(3-aminopiperidin-1-yl)(2-(2-ethylphenyl)-3-methylimidazo[1,2-a]pyridin-7-yl)methanone (31), and (R)-(3-aminopiperidin-1-yl)(2-(3-ethylbenzo[b]thiophen-2-yl)-3,4-dihydro-5-oxa-1,2a-diazaacenaphthylen-7-yl)methanone (98), or its polymorph, stereoisomer, prodrug, solvate, co-crystal, intermediate, pharmaceutically acceptable salt, metabolite or composition thereof,

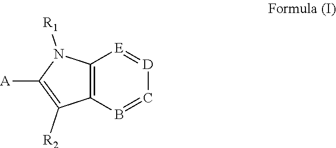

wherein,
A represents

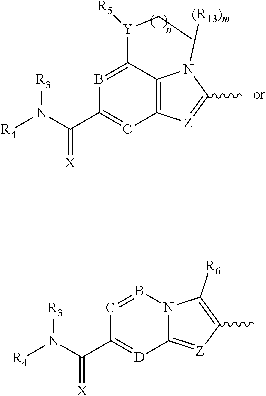

n is 0-2;
X is selected from O or S;
Y is selected from O, N, or C;
Z is selected from N or CR₇;
B is selected from N or CR₈;
C is selected from N or CR₉;
D is selected from N or CR₁₀;
E is selected from N or CR₁₁;
R₁ is selected from a group consisting of hydrogen, C₁₋₆ alkyl, C₁₋₆ haloalkyl, C₁₋₆ alkoxy, C₃₋₆ cycloalkyl, C₅₋₆ aryl, C₁₋₆ heteroaryl, C(O)C₅₋₆ aryl, C(O)C₁₋₆ heteroaryl, SO₂C₅₋₆ aryl, and SO₂C₁₋₆ heteroaryl; wherein, C₁₋₆ alkyl, C₁₋₆ alkoxy, C₃₋₆ cycloalkyl, C₅₋₆ aryl, C₁₋₆ heteroaryl, SO₂C₅₋₆ aryl, and SO₂C₁₋₆ heteroaryl, is optionally substituted with one or more groups selected from hydrogen, C₁₋₆ alkyl, C₁₋₆ alkoxy, C₃₋₈ cycloalkyl, C₅₋₆ aryl, C₁₋₆ heteroaryl, C₁₋₆ heterocyclyl, halogen, hydroxyl, —COOH, and cyano, and wherein C₁₋₆ alkyl, C₁₋₆ alkoxy, C₃₋₈ cycloalkyl, C₅₋₆ aryl, C₁₋₆ heteroaryl, C₁₋₆ heterocyclyl optionally further substituted one or more substituents selected from halogen, cyano, oxo, C₁₋₆ alkyl, C₁₋₆ alkoxy, and C₁₋₆ alkylhydroxy;
R₂, R₆, R₈, R₉, R₁₀, R₁₁, and R₁₃ are independently selected from hydrogen, C₁₋₆ alkyl, C₂₋₆ alkenyl, C₁₋₆ alkoxy, C₃₋₆ cycloalkyl, C₁₋₆ haloalkyl, C₁₋₆ haloalkoxy, C₅₋₁₀ aryl, C₁₋₆ heterocyclyl, or C₁₋₆ heteroaryl; wherein, C₁₋₆ alkyl, C₂₋₆ alkenyl, C₁₋₆ alkoxy, or C₁₋₆ heteroaryl, is optionally substituted with one or more of the groups selected from hydrogen, oxo, C₁₋₆ alkyl, C₁₋₆ alkoxy, C₁₋₆ haloalkyl, C₃₋₆ cycloalkyl, C₅₋₁₀ aryl, C₁₋₆ heterocyclyl, C₁₋₆ heteroaryl, cyano, and hydroxyl;
m is 0 to 2;
R₃ and R₄ can be taken together to form a 5-10 membered monocyclic or bicyclic saturated or unsaturated heterocyclic ring with 1-5 heteroatoms selected from N, S or O; wherein, the 5-10 membered monocyclic or bicyclic saturated or unsaturated heterocyclic ring is substituted with one or more substituents selected from a group consisting of amino, C₁₋₆ alkylamino, C₁₋₆ acylamino, —NHC(NH)CH₂Cl, —NHC(O)CH═CH—CH₂—N(CH₃)₂, C₁₋₆ alkyl, halogen, C₁₋₆ alkoxy, and hydroxyl;
R₅ is absent or is selected from a group consisting of hydrogen, C₁₋₆ alkyl, C₃₋₆ cycloalkyl, C(O)C₁₋₆ alkyl, C(O)C₁₋₆ haloalkyl, C(O)NR₁₂, C(O)C₁₋₆ alkylamino, SO₂C₁₋₆ alkyl, SO₂C₃₋₆ cycloalkyl, C₅₋₆ aryl, C₁₋₆ heteroaryl, C(O)C₅₋₆ aryl, C(O)C₁₋₆ heteroaryl, SO₂C₅₋₆ aryl, and SO₂C₁₋₆ heteroaryl; wherein, C₁₋₆ alkyl, (CO)C₁₋₆ alkyl, C(O)C₁₋₆ haloalkyl, SO₂C₅₋₆ aryl, and SO₂C₁₋₆ alkyl, is optionally substituted with C₁₋₆ alkoxy, halogen, C₅₋₆ aryl, or C₁₋₆ heteroaryl;
R₇ is selected from a group consisting of hydrogen, hydroxyl, cyano, halogen, or C₁₋₆ alkyl; and
R₁₂ is independently selected from hydrogen, and C₁₋₆ alkyl.

In an embodiment of the present disclosure, there is provided a method as described herein, wherein the compound is selected from a group consisting of:
(R)-(3-aminopiperidin-1-yl)(2-(3-bromo-1-(cyclopropylmethyl)-1H-indol-2-yl)-3-methylimidazo[1,2-a]pyridin-7-yl)methanone (1),
(R)-(3-aminopiperidin-1-yl)(2-(1-(cyclopropylmethyl)-1H-indol-2-yl)-3-methylimidazo[1,2-a]pyridin-7-yl)methanone (2),
(R)-(3-aminopiperidin-1-yl)(2-(1-benzyl-1H-indol-2-yl)-3-methylimidazo[1,2-a]pyridin-7-yl)methanone (3),
(R)-(3-aminopiperidin-1-yl)(2-(1-ethyl-1H-indol-2-yl)-3-methylimidazo[1,2-a]pyridin-7-yl)methanone (4),
(R)-(3-aminopiperidin-1-yl)(2-(1-ethyl-3-phenyl-1H-indol-2-yl)-3-methylimidazo[1,2-a]pyridin-7-yl)methanone (5),
(R)-(3-aminopyrrolidin-1-yl)(2-(1-ethyl-3-phenyl-1H-indol-2-yl)-3-methylimidazo[1,2-a]pyridin-7-yl)methanone (6),
(R)-(3-aminopyrrolidin-1-yl)(2-(1-ethyl-1H-indol-2-yl)-3-methylimidazo[1,2-a]pyridin-7-yl)methanone (7),
(R)-(3-aminopyrrolidin-1-yl)(2-(1-(cyclopropylmethyl)-1H-indol-2-yl)-3-methylimidazo[1,2-a]pyridin-7-yl)methanone (8),
(2-(1-(cyclopropylmethyl)-1H-indol-2-yl)-3-methylimidazo[1,2-a]pyridin-7-yl)(hexahydro-2H-pyrido[4,3-b][1,4]oxazin-6(5H)-yl)methanone (9),
(R)-(3-aminopiperidin-1-yl)(3-methyl-2-(1-(pyridin-4-ylmethyl)-1H-indol-2-yl)imidazo[1,2-a]pyridin-7-yl)methanone (10),
(R)-(3-aminopiperidin-1-yl)(3-methyl-2-(1-((tetrahydro-2H-pyran-4-yl)methyl)-1H-indol-2-yl)imidazo[1,2-a]pyridin-7-yl)methanone (11),
(R)-(3-aminopiperidin-1-yl)(2-(1-(4-methoxybenzyl)-1H-indol-2-yl)-3-methylimidazo[1,2-a]pyridin-7-yl)methanone (12),
(R)-(3-aminopiperidin-1-yl)(2-(1-(4-fluorobenzyl)-1H-indol-2-yl)-3-methylimidazo[1,2-a]pyridin-7-yl)methanone (13),
(R)-4-((2-(7-(3-aminopiperidine-1-carbonyl)-3-methylimidazo[1,2-a]pyridin-2-yl)-1H-indol-1-yl)methyl)benzonitrile (14),
(R)-(3-aminopiperidin-1-yl)(3-methyl-2-(1-(pyridin-3-ylmethyl)-1H-indol-2-yl)imidazo[1,2-a]pyridin-7-yl)methanone (15),
(R)-(3-aminopiperidin-1-yl)(3-methyl-2-(1-(pyridin-2-ylmethyl)-1H-indol-2-yl)imidazo[1,2-a]pyridin-7-yl)methanone (16),
(R)-(3-aminopiperidin-1-yl)(3-methyl-2-(1-(2,2,2-trifluoroethyl)-1H-indol-2-yl)imidazo[1,2-a]pyridin-7-yl)methanone (17),
(R)-4-((2-(7-(3-aminopiperidine-1-carbonyl)-3-methylimidazo[1,2-a]pyridin-2-yl)-1H-indol-1-yl)methyl)-1-methylpyridin-2(1H)-one (18),
(R)-(3-aminopiperidin-1-yl)(2-(3-ethylbenzo[b]thiophen-2-yl)-3-methylimidazo[1,2-a]pyridin-7-yl)methanone (19),
(R)-(3-aminopiperidin-1-yl)(2-(1-(4-chlorobenzyl)-1H-indol-2-yl)-3-methylimidazo[1,2-a]pyridin-7-yl)methanone (20),
(R)-(3-aminopiperidin-1-yl)(2-(1-(2-fluorobenzyl)-1H-indol-2-yl)-3-methylimidazo[1,2-a]pyridin-7-yl)methanone (21),
(R)-(3-aminopiperidin-1-yl)(2-(1-ethyl-5-phenyl-1H-pyrrol-2-yl)-3-methylimidazo[1,2-a]pyridin-7-yl)methanone (22),
(R)-(3-aminopiperidin-1-yl)(2-(1-(cyclopropylmethyl)-6-methoxy-1H-indol-2-yl)-3-methylimidazo[1,2-a]pyridin-7-yl)methanone (23),
(R)-(3-aminopiperidin-1-yl)(2-(1-ethyl-1H-pyrrolo[2,3-b]pyridin-2-yl)-3-methylimidazo[1,2-a]pyridin-7-yl)methanone (24),
(R)-(3-aminopiperidin-1-yl)(2-(1-(cyclopropylmethyl)-1H-indol-2-yl)-3,5-dimethylimidazo[1,2-a]pyridin-7-yl)methanone (25),
(R)-(3-aminopiperidin-1-yl)(2-(1-benzyl-1H-indol-2-yl)-3,5-dimethylimidazo[1,2-a]pyridin-7-yl)methanone (26),
(R)-(3-aminopiperidin-1-yl)(3,5-dimethyl-2-(1-(pyridin-2-ylmethyl)-1H-indol-2-yl)imidazo[1,2-a]pyridin-7-yl)methanone (27),
(R)-(3-aminopiperidin-1-yl)(2-(1-(cyclopropylmethyl)-6-fluoro-1H-indol-2-yl)-3-methylimidazo[1,2-a]pyridin-7-yl)methanone (28),
(R)-(3-aminopiperidin-1-yl)(2-(1-(cyclopropylmethyl)-5-fluoro-1H-indol-2-yl)-3-methylimidazo[1,2-a]pyridin-7-yl)methanone (29),
(R)-(3-aminopiperidin-1-yl)(2-(1-(cyclopropylmethyl)-7-methyl-1H-indol-2-yl)-3-methylimidazo[1,2-a]pyridin-7-yl)methanone (30),
(R)-(3-aminopiperidin-1-yl)(2-(2-ethylphenyl)-3-methylimidazo[1,2-a]pyridin-7-yl)methanone (31),
(R)-(3-aminopiperidin-1-yl)(2-(1-(cyclopropylmethyl)-1H-indol-2-yl)-3-phenylimidazo[1,2-a]pyridin-7-yl)methanone (32),
(R)-(3-aminopiperidin-1-yl)(3-cyclopropyl-2-(1-(cyclopropylmethyl)-1H-indol-2-yl)imidazo[1,2-a]pyridin-7-yl)methanone (33),
(R)-(3-aminopiperidin-1-yl)(2-(1-(cyclopropylmethyl)-1H-indol-2-yl)-3-(methoxymethyl)imidazo[1,2-a]pyridin-7-yl)methanone (34),
(R)-(3-aminopiperidin-1-yl)(2-(1-(3-fluorobenzyl)-1H-indol-2-yl)-3-methylimidazo[1,2-a]pyridin-7-yl)methanone (35),
(R)-(3-aminopiperidin-1-yl)(3-methyl-2-(1-(thiophen-3-ylmethyl)-1H-indol-2-yl)imidazo[1,2-a]pyridin-7-yl)methanone (36),
(R)-(3-aminopiperidin-1-yl)(2-(1-(furan-3-ylmethyl)-1H-indol-2-yl)-3-methylimidazo[1,2-a]pyridin-7-yl)methanone (37),
(R)-3-aminopiperidin-1-yl)(2-(1-(1-(4-fluorophenyl)ethyl)-1H-indol-2-yl)-3-methylimidazo[1,2-a]pyridin-7-yl)methanone (38),
(R)-(3-aminopiperidin-1-yl)(2-(1-ethyl-5-fluoro-1H-indol-2-yl)-3-methylimidazo[1,2-a]pyridin-7-yl)methanone (39),
(R)-(3-aminopiperidin-1-yl)(2-(1-(cyclopropylmethyl)-4-fluoro-1H-indol-2-yl)-3-methylimidazo[1,2-a]pyridin-7-yl)methanone (40),
(R)-(3-aminopiperidin-1-yl)(3-methyl-2-(1-((4-methylthiazol-2-yl)methyl)-1H-indol-2-yl)imidazo[1,2-a]pyridin-7-yl)methanone (41),
(R)-(3-aminopiperidin-1-yl)(2-(1-(3-methoxybenzyl)-1H-indol-2-yl)-3-methylimidazo[1,2-a]pyridin-7-yl)methanone (42),
(R)-(3-aminopiperidin-1-yl)(2-(5-bromo-1-(cyclopropylmethyl)-1H-indol-2-yl)-3-methylimidazo[1,2-a]pyridin-7-yl)methanone (43),
(R)-(3-aminopiperidin-1-yl)(2-(7-chloro-1-(cyclopropylmethyl)-1H-indol-2-yl)-3-methylimidazo[1,2-a]pyridin-7-yl)methanone (44),
(R)-(3-aminopiperidin-1-yl)(2-(1-(4-fluorobenzyl)-1H-indol-2-yl)-3-methylimidazo[1,2-a]pyridin-7-yl)methanethione (45),
(R)-(3-aminopiperidin-1-yl)(3-methyl-2-(1-methyl-1H-indol-2-yl)imidazo[1,2-a]pyridin-7-yl)methanone (46),
(R)-(3-aminopiperidin-1-yl)(2-(1-(cyclopropylmethyl)-7-methoxy-1H-indol-2-yl)-3-methylimidazo[1,2-a]pyridin-7-yl)methanone (47),
(R)-(3-aminopiperidin-1-yl)(2-(1-((2,4-dimethylthiazol-5-yl)methyl)-1H-indol-2-yl)-3-methylimidazo[1,2-a]pyridin-7-yl)methanone (48),
(R)-(3-aminopiperidin-1-yl)(3-methyl-2-(1-((2-methylthiazol-5-yl)methyl)-1H-indol-2-yl)imidazo[1,2-a]pyridin-7-yl)methanone (49),
(R)-(3-aminopiperidin-1-yl) (2-(1-(3-methoxybenzyl)-1H-indol-2-yl)-3-methylimidazo[1,2-a]pyridin-7-yl)methanone trifluoroacetic acid salt (50),
(R)-(3-aminopiperidin-1-yl)(2-(6-methoxy-1-(pyridin-3-ylmethyl)-1H-indol-2-yl)-3-methylimidazo[1,2-a]pyridin-7-yl)methanone (51),
(R)-(3-aminopiperidin-1-yl)(2-(1-(cyclopropylmethyl)-6-fluoro-1H-indol-2-yl)-3-methylimidazo[1,2-a]pyridin-7-yl)methanone trifluoroacetic acid salt (52),
(R)-(3-aminopiperidin-1-yl)(2-(5-fluoro-1-(3-methoxybenzyl)-1H-indol-2-yl)-3-methylimidazo[1,2-a]pyridin-7-yl)methanone (53),
(R)-(3-aminopiperidin-1-yl)(2-(6-fluoro-1-(4-fluorobenzyl)-1H-indol-2-yl)-3-methylimidazo[1,2-a]pyridin-7-yl)methanone (54),
(R)-(3-aminopiperidin-1-yl)(2-(5,6-difluoro-1-(3-methoxybenzyl)-1H-indol-2-yl)-3-methylimidazo[1,2-a]pyridin-7-yl)methanone (55),
(R,E)-4-(dimethylamino)-N-(1-(2-(1-(4-fluorobenzyl)-1H-indol-2-yl)-3-methylimidazo[1,2-a]pyridine-7-carbonyl)piperidin-3-yl)but-2-enamide (56),
(R)-(3-aminopiperidin-1-yl)(3-methyl-2-(1-(pyrazin-2-ylmethyl)-1H-indol-2-yl)imidazo[1,2-a]pyridin-7-yl)methanone (57),
(R)-(3-aminopiperidin-1-yl)(3-methyl-2-(1-(pyrimidin-5-ylmethyl)-1H-indol-2-yl)imidazo[1,2-a]pyridin-7-yl)methanone trifluoroacetic acid salt (58),
(R)-(3-aminopiperidin-1-yl)(3-methyl-2-(1-(pyridazin-3-ylmethyl)-1H-indol-2-yl)imidazo[1,2-a]pyridin-7-yl)methanone (59),
(R)-(3-aminopiperidin-1-yl)(2-(1-isobutyl-1H-indol-2-yl)-3-methylimidazo[1,2-a]pyridin-7-yl)methanone (60),
(R)-(3-aminopiperidin-1-yl)(2-(1-(cyclobutylmethyl)-1H-indol-2-yl)-3-methylimidazo[1,2-a]pyridin-7-yl)methanone (61),
(R)-(3-aminopiperidin-1-yl)(2-(1-((3-fluoropyridin-2-yl)methyl)-1H-indol-2-yl)-3-methylimidazo[1,2-a]pyridin-7-yl)methanone (62),
(R)-(3-aminopiperidin-1-yl)(2-(1-((5-methoxypyridin-2-yl)methyl)-1H-indol-2-yl)-3-methylimidazo[1,2-a]pyridin-7-yl)methanone (63),
(R)-(3-aminopiperidin-1-yl)(2-(1-(2-methoxyethyl)-1H-indol-2-yl)-3-methylimidazo[1,2-a]pyridin-7-yl)methanone (64),
(R)-(3-aminopiperidin-1-yl)(2-(1-(2-hydroxyethyl)-1H-indol-2-yl)-3-methylimidazo[1,2-a]pyridin-7-yl)methanone (65),
(R)-(3-aminopiperidin-1-yl)(2-(6-methoxy-1-(pyridin-4-ylmethyl)-1H-indol-2-yl)-3-methylimidazo[1,2-a]pyridin-7-yl)methanone (66),
(R)-(3-aminopiperidin-1-yl)(2-(1-ethyl-6-methoxy-1H-indol-2-yl)-3-methylimidazo[1,2-a]pyridin-7-yl)methanone (67),
(R)-(3-aminopiperidin-1-yl)(2-(6-fluoro-1-(pyridin-4-ylmethyl)-1H-indol-2-yl)-3-methylimidazo[1,2-a]pyridin-7-yl)methanone (68),
(R)-(3-aminopiperidin-1-yl)(2-(6-fluoro-1-(pyridin-3-ylmethyl)-1H-indol-2-yl)-3-methylimidazo[1,2-a]pyridin-7-yl)methanone (69),
(R)-(3-aminopiperidin-1-yl)(2-(6-fluoro-1-(3-methoxybenzyl)-1H-indol-2-yl)-3-methylimidazo[1,2-a]pyridin-7-yl)methanone (70),
(R)-(3-aminopiperidin-1-yl)(2-(6-fluoro-1-(4-methoxybenzyl)-1H-indol-2-yl)-3-methylimidazo[1,2-a]pyridin-7-yl)methanone (71),
(R)-(3-aminopiperidin-1-yl)(2-(1-ethyl-6-fluoro-1H-indol-2-yl)-3-methylimidazo[1,2-a]pyridin-7-yl)methanone (72),
(R)-(3-aminopiperidin-1-yl)(2-(6-fluoro-1-isobutyl-1H-indol-2-yl)-3-methylimidazo[1,2-a]pyridin-7-yl)methanone (73),
(R)-(3-aminopiperidin-1-yl)(2-(5-fluoro-1-(pyridin-4-ylmethyl)-1H-indol-2-yl)-3-methylimidazo[1,2-a]pyridin-7-yl)methanone (74),
(R)-(3-aminopiperidin-1-yl)(2-(5-fluoro-1-(4-methoxybenzyl)-1H-indol-2-yl)-3-methylimidazo[1,2-a]pyridin-7-yl)methanone (75),
(R)-(3-aminopiperidin-1-yl)(2-(5-fluoro-1-(4-fluorobenzyl)-1H-indol-2-yl)-3-methylimidazo[1,2-a]pyridin-7-yl)methanone (76),
(R)-(3-aminopiperidin-1-yl)(2-(7-chloro-1-(4-fluorobenzyl)-1H-indol-2-yl)-3-methylimidazo[1,2-a]pyridin-7-yl)methanone (77),
(R)-(3-aminopiperidin-1-yl)(2-(1-(2,2-difluoroethyl)-1H-indol-2-yl)-3-methylimidazo[1,2-a]pyridin-7-yl)methanone trifluoroacetic acid salt (78),
(R)-(3-aminopiperidin-1-yl)(2-(1-((5-fluoropyridin-2-yl)methyl)-1H-indol-2-yl)-3-methylimidazo[1,2-a]pyridin-7-yl)methanone (79),
(R)-(3-aminopiperidin-1-yl)(2-(1-(4-fluorobenzyl)-6-methoxy-1H-indol-2-yl)-3-methylimidazo[1,2-a]pyridin-7-yl)methanone (80),
(R)-(3-aminopiperidin-1-yl)(2-(1-(4-(hydroxymethyl)benzyl)-1H-indol-2-yl)-3-methylimidazo[1,2-a]pyridin-7-yl)methanone (81),
(R)-(3-aminopiperidin-1-yl)(2-(1-isobutyl-6-methoxy-1H-indol-2-yl)-3-methylimidazo[1,2-a]pyridin-7-yl)methanone (82),
(R)-(3-aminopiperidin-1-yl)(2-(1-(2,2-difluoroethyl)-6-methoxy-1H-indol-2-yl)-3-methylimidazo[1,2-a]pyridin-7-yl)methanone (83),
(R)-(3-aminopiperidin-1-yl)(2-(5-fluoro-1-isobutyl-1H-indol-2-yl)-3-methylimidazo[1,2-a]pyridin-7-yl)methanone (84),
(R)-(3-aminopiperidin-1-yl)(2-(1-(4-fluoro-3-methoxybenzyl)-1H-indol-2-yl)-3-methylimidazo[1,2-a]pyridin-7-yl)methanone (85).
(R)-(3-aminopiperidin-1-yl)(2-(1-(cyclopropylmethyl)-6-methoxy-1H-indol-2-yl)-3-(methoxymethyl)imidazo[1,2-a]pyridin-7-yl)methanone (86)
(R)-(3-aminopiperidin-1-yl)(2-(1-(cyclopropylmethyl)-1H-indol-2-yl)-3,4-dihydro-5-oxa-1,2a-diazaacenaphthylen-7-yl)methanone (87),
(R)-(3-aminopyrrolidin-1-yl)(2-(1-(cyclopropylmethyl)-1H-indol-2-yl)-3,4-dihydro-5-oxa-1,2a-diazaacenaphthylen-7-yl)methanone (88),
(R)-(3-aminopiperidin-1-yl)(2-(1-ethyl-1H-indol-2-yl)-3,4-dihydro-5-oxa-1,2a-diazaacenaphthylen-7-yl)methanone (89),
(2-(aminomethyl)piperidin-1-yl)(2-(1-(cyclopropylmethyl)-1H-indol-2-yl)-3,4-dihydro-5-oxa-1,2a-diazaacenaphthylen-7-yl)methanone (90),
(R)-(3-aminopiperidin-1-yl)(2-(1-ethyl-1H-pyrrolo[2,3-b]pyridin-2-yl)-3,4-dihydro-5-oxa-1,2a-diazaacenaphthylen-7-yl)methanone (91),
(R)-(3-aminopiperidin-1-yl)(1-(1-(cyclopropylmethyl)-1H-indol-2-yl)-8,9-dihydro-7H-6-oxa-2,9a-diazabenzo[cd]azulen-4-yl)methanone (92),
(R)-(3-aminopyrrolidin-1-yl)(1-(1-(cyclopropylmethyl)-1H-indol-2-yl)-8,9-dihydro-7H-6-oxa-2,9a-diazabenzo[cd]azulen-4-yl)methanone (93),
(R)-(3-aminopiperidin-1-yl)(2-(1-(cyclopropylmethyl)-7-methyl-1H-indol-2-yl)-3,4-dihydro-5-oxa-1,2a-diazaacenaphthylen-7-yl)methanone (94),
(R)-(3-aminopiperidin-1-yl)(2-(1-(cyclopropylmethyl)-5-fluoro-1H-indol-2-yl)-3,4-dihydro-5-oxa-1,2a-diazaacenaphthylen-7-yl)methanone (95),
(R)-(3-aminopiperidin-1-yl)(2-(1-(pyridin-4-ylmethyl)-1H-indol-2-yl)-3,4-dihydro-5-oxa-1,2a-diazaacenaphthylen-7-yl)methanone (96),
(R)-(3-aminopiperidin-1-yl)(2-(1-(pyridin-2-ylmethyl)-1H-indol-2-yl)-3,4-dihydro-5-oxa-1,2a-diazaacenaphthylen-7-yl)methanone (97),
(R)-(3-aminopiperidin-1-yl)(2-(3-ethylbenzo[b]thiophen-2-yl)-3,4-dihydro-5-oxa-1,2a-diazaacenaphthylen-7-yl)methanone (98),
(R)-(3-aminopiperidin-1-yl)(2-(1-(4-chlorobenzyl)-1H-indol-2-yl)-3,4-dihydro-5-oxa-1,2a-diazaacenaphthylen-7-yl)methanone (99),
(R)-(3-aminopiperidin-1-yl)(2-(1-(2-fluorobenzyl)-1H-indol-2-yl)-3,4-dihydro-5-oxa-1,2a-diazaacenaphthylen-7-yl)methanone (100),
(R)-(3-aminopiperidin-1-yl)(2-(1-(4-fluorobenzyl)-1H-indol-2-yl)-3,4-dihydro-5-oxa-1,2a-diazaacenaphthylen-7-yl)methanone (101),
(R)-(3-aminopiperidin-1-yl)(2-(1-(pyridin-3-ylmethyl)-1H-indol-2-yl)-3,4-dihydro-5-oxa-1,2a-diazaacenaphthylen-7-yl)methanone (102),
(R)-(3-aminopiperidin-1-yl)(2-(1-(cyclopropylmethyl)-5,6-dimethoxy-1H-indol-2-yl)-3,4-dihydro-5-oxa-1,2a-diazaacenaphthylen-7-yl)methanone (103),
(R)-(3-aminopiperidin-1-yl)(2-(1-benzyl-1H-indol-2-yl)-3,4-dihydro-5-oxa-1,2a-diazaacenaphthylen-7-yl)methanone (104),
(R)-(3-aminopiperidin-1-yl)(2-(1-(4-methoxybenzyl)-1H-indol-2-yl)-3,4-dihydro-5-oxa-1,2a-diazaacenaphthylen-7-yl)methanone (105),
(R)-(3-aminopiperidin-1-yl)(2-(1-(2-methoxyethyl)-1H-indol-2-yl)-3,4-dihydro-5-oxa-1,2a-diazaacenaphthylen-7-yl)methanone (106),
(R)-(3-aminopiperidin-1-yl)(2-(6-methoxy-1-(2-methoxyethyl)-1H-indol-2-yl)-3,4-dihydro-5-oxa-1,2a-diazaacenaphthylen-7-yl)methanone (107),
(R)-(3-aminopiperidin-1-yl)(2-(1-(2-hydroxyethyl)-1H-indol-2-yl)-3,4-dihydro-5-oxa-1,2a-diazaacenaphthylen-7-yl)methanone (108),
(R)-(3-aminopiperidin-1-yl)(2-(1-(cyclopropylmethyl)-6-methoxy-1H-indol-2-yl)-3,4-dihydro-5-oxa-1,2a-diazaacenaphthylen-7-yl)methanone (109),
(R)-(3-aminopiperidin-1-yl)(2-(1-((3-fluoropyridin-4-yl)methyl)-1H-indol-2-yl)-3,4-dihydro-5-oxa-1,2a-diazaacenaphthylen-7-yl)methanone (110),
(R)-(3-aminopiperidin-1-yl)(2-(1-(pyrazin-2-ylmethyl)-1H-indol-2-yl)-3,4-dihydro-5-oxa-1,2a-diazaacenaphthylen-7-yl)methanone (111),
(R)-(3-aminopiperidin-1-yl)(2-(1-((3-fluoropyridin-2-yl)methyl)-1H-indol-2-yl)-3,4-dihydro-5-oxa-1,2a-diazaacenaphthylen-7-yl)methanone (112),
(R)-(3-aminopiperidin-1-yl)(2-(1-(pyrimidin-2-ylmethyl)-1H-indol-2-yl)-3,4-dihydro-5-oxa-1,2a-diazaacenaphthylen-7-yl)methanone (113),
(R)-(3-aminopiperidin-1-yl)(2-(1-(cyclopropylmethyl)-6-fluoro-1H-indol-2-yl)-3,4-dihydro-5-oxa-1,2a-diazaacenaphthylen-7-yl)methanone (114),
(R)-(3-aminopiperidin-1-yl)(2-(1-(pyrimidin-5-ylmethyl)-1H-indol-2-yl)-3,4-dihydro-5-oxa-1,2a-diazaacenaphthylen-7-yl)methanone (115),
(R)-(3-aminopiperidin-1-yl)(2-(1-(pyridazin-3-ylmethyl)-1H-indol-2-yl)-3,4-dihydro-5-oxa-1,2a-diazaacenaphthylen-7-yl)methanone (116),
(R)-(3-aminopiperidin-1-yl)(2-(1-isobutyl-1H-indol-2-yl)-3,4-dihydro-5-oxa-1,2a-diazaacenaphthylen-7-yl)methanone (117),
(R)-(3-aminopiperidin-1-yl)(2-(1-(cyclopropylmethyl)-5,6-difluoro-1H-indol-2-yl)-3,4-dihydro-5-oxa-1,2a-diazaacenaphthylen-7-yl)methanone (118),
(R)-(3-aminopiperidin-1-yl)(2-(1-((3-fluoropyridin-2-yl)methyl)-6-methoxy-1H-indol-2-yl)-3,4-dihydro-5-oxa-1,2a-diazaacenaphthylen-7-yl)methanone (119),
(R)-(3-aminopiperidin-1-yl)(2-(7-chloro-1-((3-fluoropyridin-2-yl)methyl)-1H-indol-2-yl)-3,4-dihydro-5-oxa-1,2a-diazaacenaphthylen-7-yl)methanone (120),
(R)-(3-aminopiperidin-1-yl)(2-(6-fluoro-1-((3-fluoropyridin-2-yl)methyl)-1H-indol-2-yl)-3,4-dihydro-5-oxa-1,2a-diazaacenaphthylen-7-yl)methanone (121),
(R)-(3-aminopiperidin-1-yl)(2-(7-chloro-1-(2-methoxyethyl)-1H-indol-2-yl)-3,4-dihydro-5-oxa-1,2a-diazaacenaphthylen-7-yl)methanone (122),
(R)-(3-aminopiperidin-1-yl)(2-(1-(4-(hydroxymethyl)benzyl)-1H-indol-2-yl)-3,4-dihydro-5-oxa-1,2a-diazaacenaphthylen-7-yl)methanone trifluoroacetic acid salt (123),
(R,E)-N-(1-(2-(1-(cyclopropylmethyl)-1H-indol-2-yl)-3,4-dihydro-5-oxa-1,2a-diazaacenaphthylene-7-carbonyl)piperidin-3-yl)-4-(dimethylamino)but-2-enamide trifluoroacetic acid salt (124),
(R)-(3-aminopiperidin-1-yl)(2-(7-chloro-1-(4-methoxybenzyl)-1H-indol-2-yl)-3,4-dihydro-5-oxa-1,2a-diazaacenaphthylen-7-yl)methanone (125),
(R)-(3-aminopiperidin-1-yl)(2-(1-((tetrahydro-2H-pyran-4-yl)methyl)-1H-indol-2-yl)-3,4-dihydro-5-oxa-1,2a-diazaacenaphthylen-7-yl)methanone (126),
(R)-(3-aminopiperidin-1-yl)(2-(1-(cyclobutylmethyl)-1H-indol-2-yl)-3,4-dihydro-5-oxa-1,2a-diazaacenaphthylen-7-yl)methanone (127),
(R)-2-(2-(7-(3-aminopiperidine-1-carbonyl)-3,4-dihydro-5-ox1,2adiazaacenaphthylen-2-yl)-1H-indol-1-yl)acetic acid (128),
(R)-(3-aminopiperidin-1-yl)(2-(1-(piperidin-4-ylmethyl)-1H-indol-2-yl)-3,4-dihydro-5-oxa-1,2a-diazaacenaphthylen-7-yl)methanone (129),
(R)-(3-aminopiperidin-1-yl)(2-(1-(oxetan-3-ylmethyl)-1H-indol-2-yl)-3,4-dihydro-5-oxa-1,2a-diazaacenaphthylen-7-yl)methanone (130),
(R)-(3-aminopiperidin-1-yl)(2-(1-((1-methylpiperidin-4-yl)methyl)-1H-indol-2-yl)-3,4-dihydro-5-oxa-1,2a-diazaacenaphthylen-7-yl)methanone (131),
(R)-(3-aminopiperidin-1-yl)(2-(5-fluoro-1-(4-methoxybenzyl)-1H-indol-2-yl)-3,4-dihydro-5-oxa-1,2a-diazaacenaphthylen-7-yl)methanone (132),
(R)-(3-aminopiperidin-1-yl)(2-(1-(2,2-difluoroethyl)-1H-indol-2-yl)-3,4-dihydro-5-oxa-1,2a-diazaacenaphthylen-7-yl)methanone (133),
(R)-(3-aminopiperidin-1-yl)(2-(5-fluoro-1-(2-methoxyethyl)-1H-indol-2-yl)-3,4-dihydro-5-oxa-1,2a-diazaacenaphthylen-7-yl)methanone (134),
(R)-(3-aminopiperidin-1-yl)(2-(6-fluoro-1-(4-fluorobenzyl)-1H-indol-2-yl)-3,4-dihydro-5-oxa-1,2a-diazaacenaphthylen-7-yl)methanone (135),
(R)-(3-aminopiperidin-1-yl)(2-(6-fluoro-1-(4-methoxybenzyl)-1H-indol-2-yl)-3,4-dihydro-5-oxa-1,2a-diazaacenaphthylen-7-yl)methanone (136),
(R)-(3-aminopiperidin-1-yl)(2-(1-(4-fluorobenzyl)-6-methoxy-1H-indol-2-yl)-3,4-dihydro-5-oxa-1,2a-diazaacenaphthylen-7-yl)methanone (137),
3-aminopiperidin-1-yl)(2-(6-fluoro-1-(2-methoxyethyl)-1H-indol-2-yl)-3,4-dihydro-5-oxa-1,2a-diazaacenaphthylen-7-yl)methanone (138),
(R)-(3-aminopiperidin-1-yl)(2-(7-chloro-1-(cyclobutylmethyl)-1H-indol-2-yl)-3,4-dihydro-5-oxa-1,2a-diazaacenaphthylen-7-yl)methanone (139),
(R)-(3-aminopiperidin-1-yl)(2-(5,6-difluoro-1-(2-methoxyethyl)-1H-indol-2-yl)-3,4-dihydro-5-oxa-1,2a-diazaacenaphthylen-7-yl)methanone (140),
(R)-(3-aminopiperidin-1-yl)(2-(7-chloro-1-isobutyl-1H-indol-2-yl)-3,4-dihydro-5-oxa-1,2a-diazaacenaphthylen-7-yl)methanone (141),
(R)-(3-aminopiperidin-1-yl)(2-(7-chloro-1-(2,2-difluoroethyl)-1H-indol-2-yl)-3,4-dihydro-5-oxa-1,2a-diazaacenaphthylen-7-yl)methanone (142),
(R)-(3-aminopiperidin-1-yl)(2-(7-chloro-1-(cyclopropylmethyl)-1H-indol-2-yl)-3,4-dihydro-5-oxa-1,2a-diazaacenaphthylen-7-yl)methanone (143),
(R,E)-(3-aminopiperidin-1-yl)(2-(1-(cyclopropylmethyl)-5-styryl-1H-indol-2-yl)-3,4-dihydro-5-oxa-1,2a-diazaacenaphthylen-7-yl)methanone (144),
(R)-(3-aminopiperidin-1-yl)(2-(1-((4-methylthiazol-2-yl)methyl)-1H-indol-2-yl)-3,4-dihydro-5-oxa-1,2a-diazaacenaphthylen-7-yl)methanone (145),
(R)-(3-aminopiperidin-1-yl)(2-(1-(cyclopropylmethyl)-5-methoxy-1H-indol-2-yl)-3,4-dihydro-5-oxa-1,2a-diazaacenaphthylen-7-yl)methanone (146),
(R)-(3-aminopiperidin-1-yl)(2-(1-(cyclopropylmethyl)-6-(hydroxymethyl)-1H-indol-2-yl)-3,4-dihydro-5-oxa-1,2a-diazaacenaphthylen-7-yl) methanone (147),
(R)-(3-aminopiperidin-1-yl)(2-(1-(cyclopropylmethyl)-1H-indol-2-yl)-3,3-dimethyl-3,4-dihydro-5-oxa-1,2a-diazaacenaphthylen-7-yl)methanone (148),
(R)-(3-aminopiperidin-1-yl)(2-(1-(cyclopropylmethyl)-1H-indol-2-yl)-5,6-dihydro-4H-imidazo[1,5,4-de]quinoxalin-8-yl)methanone (149),
(R)-(3-aminopiperidin-1-yl)(2-(1-(cyclopropylmethyl)-5-fluoro-1H-indol-2-yl)-5,6-dihydro-4H-imidazo[1,5,4-de]quinoxalin-8-yl)methanone (150),
(R)-(3-aminopiperidin-1-yl)(2-(1-(cyclopropylmethyl)-6-fluoro-1H-indol-2-yl)-5,6-dihydro-4H-imidazo[1,5,4-de]quinoxalin-8-yl)methanone (151),
(R)-(3-aminopiperidin-1-yl)(2-(1-(pyridin-3-ylmethyl)-1H-indol-2-yl)-5,6-dihydro-4H-imidazo[1,5,4-de]quinoxalin-8-yl)methanone (152),
(R)-(3-aminopiperidin-1-yl)(2-(5-bromo-1-(cyclopropylmethyl)-1H-indol-2-yl)-5,6-dihydro-4H-imidazo[1,5,4-de]quinoxalin-8-yl)methanone (153),
(R)-(3-aminopiperidin-1-yl)(2-(1-(cyclopropylmethyl)-5-(pyridin-3-yl)-1H-indol-2-yl)-5,6-dihydro-4H-imidazo[1,5,4-de]quinoxalin-8-yl)methanone (154),
(R)-(3-aminopiperidin-1-yl)(2-(1-(pyridin-2-ylmethyl)-1H-indol-2-yl)-5,6-dihydro-4H-imidazo[1,5,4-de]quinoxalin-8-yl)methanone (155),
(R)-(3-aminopiperidin-1-yl)(2-(1-(2-fluorobenzyl)-1H-indol-2-yl)-5,6-dihydro-4H-imidazo[1,5,4-de]quinoxalin-8-yl)methanone (156),
(R)-(3-aminopiperidin-1-yl)(2-(1-(pyridin-4-ylmethyl)-1H-indol-2-yl)-5,6-dihydro-4H-imidazo[1,5,4-de]quinoxalin-8-yl)methanone (157),
(R)-(3-aminopiperidin-1-yl)(2-(1-(cyclopropylmethyl)-1H-indol-2-yl)-6-methyl-5,6-dihydro-4H-imidazo[1,5,4-de]quinoxalin-8-yl)methanone (158),
(R)-(3-aminopiperidin-1-yl)(2-(1-(cyclopropylmethyl)-6-methoxy-1H-indol-2-yl)-5,6-dihydro-4H-imidazo[1,5,4-de]quinoxalin-8-yl)methanone (159),
(R)-(3-aminopiperidin-1-yl)(2-(1-(cyclopropylmethyl)-6-methoxy-1H-indol-2-yl)-6-methyl-5,6-dihydro-4H-imidazo[1,5,4-de]quinoxalin-8-yl)methanone (160),
(R)-(3-aminopiperidin-1-yl)(2-(1-(cyclopropylmethyl)-5-fluoro-1H-indol-2-yl)-6-methyl-5,6-dihydro-4H-imidazo[1,5,4-de]quinoxalin-8-yl)methanone (161),
(R)-(3-aminopiperidin-1-yl)(2-(1-(cyclopropylmethyl)-1H-indol-2-yl)-6-(methylsulfonyl)-5,6-dihydro-4H-imidazo[1,5,4-de]quinoxalin-8-yl)methanone (162),
(R)-(3-aminopiperidin-1-yl)(2-(1-(cyclopropylmethyl)-5,6-difluoro-1H-indol-2-yl)-5,6-dihydro-4H-imidazo[1,5,4-de]quinoxalin-8-yl)methanone (163),
(R)-(3-aminopiperidin-1-yl)(6-cyclopropyl-2-(1-(cyclopropylmethyl)-1H-indol-2-yl)-5,6-dihydro-4H-imidazo[1,5,4-de]quinoxalin-8-yl)methanone (164),
(R)-(3-aminopiperidin-1-yl)(2-(1-(cyclopropylmethyl)-1H-indol-2-yl)-6-(phenethylsulfonyl)-5,6-dihydro-4H-imidazo[1,5,4-de]quinoxalin-8-yl)methanone (165),
(R)-(3-aminopiperidin-1-yl)(2-(1-(cyclopropylmethyl)-4-fluoro-1H-indol-2-yl)-5,6-dihydro-4H-imidazo[1,5,4-de]quinoxalin-8-yl)methanone (166),
(R)-(3-aminopiperidin-1-yl)(6-((4-chlorophenyl)sulfonyl)-2-(1-(cyclopropylmethyl)-1H-indol-2-yl)-5,6-dihydro-4H-imidazo[1,5,4-de]quinoxalin-8-yl)methanone (167),
(R)-(3-aminopiperidin-1-yl)(2-(1-(cyclopropylmethyl)-1H-indol-2-yl)-6-(cyclopropylsulfonyl)-5,6-dihydro-4H-imidazo[1,5,4-de]quinoxalin-8-yl)methanone (168),
(R)-(3-aminopiperidin-1-yl)(2-(1-(cyclopropylmethyl)-1H-indol-2-yl)-6-((2-ethoxyethyl)sulfonyl)-5,6-dihydro-4H-imidazo[1,5,4-de]quinoxalin-8-yl)methanone (169),
(R)-(3-aminopiperidin-1-yl)(2-(1-(cyclopropylmethyl)-7-methyl-1H-indol-2-yl)-5,6-dihydro-4H-imidazo[1,5,4-de]quinoxalin-8-yl)methanone (170),
(R)-1-(8-(3-aminopiperidine-1-carbonyl)-2-(1-(cyclopropylmethyl)-1H-indol-2-yl)-4,5-dihydro-6H-imidazo[1,5,4-de]quinoxalin-6-yl)ethan-1-one (171),
(R)-(3-aminopiperidin-1-yl)(2-(1-(cyclopropylmethyl)-1H-indol-2-yl)-5,6-dihydro-4H-imidazo[1,5,4-de]quinoxalin-8-yl)methanethione (172),
(R)-(3-aminopiperidin-1-yl)(2-(7-chloro-1-(cyclopropylmethyl)-6-fluoro-1H-indol-2-yl)-3,4-dihydro-5-oxa-1,2a-diazaacenaphthylen-7-yl)methanone (173), and
(R)-(3-aminopiperidin-1-yl)(2-(7-chloro-1-(pyrimidin-5-ylmethyl)-1H-indol-2-yl)-3,4-dihydro-5-oxa-1,2a-diazaacenaphthylen-7-yl)methanone (174).

In an embodiment of the present disclosure, there is provided a method as described herein, wherein the disease or disorder or condition is selected from acute respiratory distress syndrome (ARDS), COVID-19 (corona virus disease of 2019) related lung infection, multi-organ failure or multi-organ dysfunction syndrome from ARDS, anti neutrophil cytoplasmic cntibody (ANCA) associated vasculitis (AAV), cancer, cancer metastasis, immune disorder, immunodeficiency disorder, inflammatory disorder, transplant rejection, arthritis, rheumatoid arthritis (RA), hidradenitis suppurativa (HS), diabetes, thrombosis, stroke, chronic obstructive pulmonary disease (COPD), cystic fibrosis, pulmonary disease, inflammatory bowel disease (IBS), crohn's disease, ulcerative colitis, indeterminate colitis, or alzheimer's Disease.

In an embodiment of the present disclosure, there is provided a method as described herein, wherein a disease or disorder or condition is selected from cancer, cancer metastasis, immune disorder, immunodeficiency disorder, inflammatory disorder and transplant rejection.

In an embodiment of the present disclosure, there is provided a method as described herein, wherein the disease or disorder or condition is cancer.

In yet another embodiment of the present disclosure, there is provided a method as described herein, wherein the disease or disorder or condition is selected from systemic inflammatory response syndrome, decreasing Neutrophil Extra cellular traps (NETs) release, acute lung injury (ALI), acute respiratory distress syndrome (ARDS), COVID-19 related lung infection, multi-organ failure or multi-organ dysfunction syndrome from ARDS, Anti Neutrophil Cytoplasmic Antibody (ANCA) associated vasculitis (AAV), Hidradenitis suppurativa (HS), sepsis, infections, or cytokine storms induced by drugs or any agent, ischemic or haemorrhagic stroke, ischemic or drug-induced haemorrhagic transformation in the brain, haemorrhagic encephalopathy, traumatic brain injury, anoxic brain injury, chronic kidney disease, diabetes, deep vein thrombosis, systemic microthrombosis, atherosclerotic thrombosis, thromboembolism, systemic lupus erythematosus (SLE), rheumatoid arthritis, COPD; cystic fibrosis, pulmonary disease, inflammatory bowel disease (IBS), Crohn's disease, ulcerative colitis, indeterminate colitis or Alzheimer's Disease. In one another embodiment of the present disclosure, wherein the disease or disorder or condition is selected from systemic inflammatory response syndrome, decreasing Neutrophil Extra cellular traps (NETs) release, acute lung injury (ALI), acute respiratory distress syndrome (ARDS), COVID-19 related lung infection, multi-organ failure or multi-organ dysfunction syndrome from ARDS, Anti Neutrophil Cytoplasmic Antibody (ANCA) associated vasculitis (AAV), cystic fibrosis, or ulcerative colitis.

In further embodiment of the present disclosure, there is provided a method as described herein, wherein the disease or disorder or condition is cancer. In yet another embodiment of the present disclosure, there is provided a method as described herein, wherein the disease or disorder or condition is lung cancer. In yet another embodiment of the present disclosure, there is provided a method as described herein, wherein the disease or disorder or condition is breast cancer.

In yet another embodiment of the present disclosure, there is provided a method as described herein, wherein the cancer is selected from breast cancer, prostate cancer, pancreatic cancer, gastric cancer, lung cancer, colon cancer, rectal cancer, esophagus cancer, duodenal cancer, tongue cancer, pharyngeal cancer, brain tumor, neurinoma, clear cell carcinoma, non-small cell lung cancer, small cell lung cancer, liver cancer, kidney cancer, bile duct cancer, uterine body cancer, cervical cancer, ovarian cancer, urinary bladder, skin cancer, hemangioma, malignant lymphoma, malignant melanoma, thyroid cancer, bone tumor, vascular fibroma, glioblastoma, sarcoma, neuroendocrine tumors, retinoblastoma, penile cancer, pediatric solid cancer, renal cell carcinoma, lymphoma, myeloma and leukemia (including, for example acute myelogenous leukemia (AML), chronic myelogenous leukemia (CML), chronic neutrophilic leukemia, chronic eosinophilic leukemia, chronic lymphocytic leukemia (CLL), acute lymphoblastic leukemia (ALL), hairy cell leukemia, cutaneous T-cell lymphoma (CTCL), multiple myeloma (MM), Myeloproliferative neoplasms (MPN), a disease category that includes polycythemia vera (PV), essential thrombocythemia, essential thrombocytosis (ET) and myelofibrosis (MF), chronic myelogenous leukemia (CML), chronic neutrophilic leukemia (CNL), or chronic eosinophilic leukemia (CEL).

In further embodiment of the present disclosure, there is provided a method as described herein, wherein the disease or disorder or condition is cancer metastasis.

In yet another embodiment of the present disclosure, there is provided a method as described herein, wherein the cancer metastasis is liver cancer metastasis, lung cancer metastasis, and omentum cancer metastasis.

In yet another embodiment of the present disclosure, there is provided a method as described herein, wherein the disease or disorder or condition is cancer metastasis, and cancer metastasis is selected from liver cancer metastasis, lung cancer metastasis, and omentum cancer metastasis.

In further embodiment of the present disclosure, there is provided a method as described herein, wherein the cancer metastasis is liver cancer metastasis originating from colorectal cancer and pancreatic cancer, lung cancer metastasis originating from breast cancer, and omentum cancer metastasis originating from ovarian cancer.

In further embodiment of the present disclosure, there is provided a method as described herein, wherein the composition is a pharmaceutical composition comprising the compound as disclosed herein, together with a pharmaceutically acceptable carrier, optionally in combination with one or more other pharmaceutical compositions.

In yet another embodiment of the present disclosure, there is provided a method as described herein, wherein the composition is in the form selected from a group consisting of a tablet, capsule, powder, syrup, solution, aerosol and suspension. In further embodiment of the present disclosure, there is provided a method as described herein, wherein said method comprising administering a combination of the compounds as disclosed herein, its polymorph, stereoisomer, prodrug, solvate, co-crystal, intermediate, pharmaceutically acceptable salt, metabolite, or composition thereof; or the pharmaceutical composition comprising the compound together with a pharmaceutically acceptable carrier, with other clinically relevant cytotoxic agents or non-cytotoxic agents to a subject in need thereof.

In yet another embodiment of the present disclosure, there is provided a method as described herein, wherein said method comprising administering a combination of the compounds as disclosed herein, its polymorph, stereoisomer, prodrug, solvate, co-crystal, intermediate, pharmaceutically acceptable salt, metabolite, or composition thereof; or the pharmaceutical composition comprising the compound together with a pharmaceutically acceptable carrier, with other clinically relevant immune modulator agents or anti-inflammatory agents to a subject in need of thereof.

In an embodiment of the present disclosure, there is provided a compound as described above, for use in the treatment of a disease or disorder or condition ameliorated by inhibition of NETosis.

In an embodiment of the present disclosure, there is provided a compound, its polymorph, stereoisomer, prodrug, solvate, co-crystal, intermediate, pharmaceutically acceptable salt, metabolite or composition thereof, for use in the treatment of a disease or disorder or condition ameliorated by inhibition of NETosis as described herein, wherein the compound is selected from a group consisting of:
(R)-(3-aminopiperidin-1-yl)(2-(3-bromo-1-(cyclopropylmethyl)-1H-indol-2-yl)-3-methylimidazo[1,2-a]pyridin-7-yl)methanone (1),
(R)-(3-aminopiperidin-1-yl)(2-(1-(cyclopropylmethyl)-1H-indol-2-yl)-3-methylimidazo[1,2-a]pyridin-7-yl)methanone (2),
(R)-(3-aminopiperidin-1-yl)(2-(1-benzyl-1H-indol-2-yl)-3-methylimidazo[1,2-a]pyridin-7-yl)methanone (3),
(R)-(3-aminopiperidin-1-yl)(2-(1-ethyl-1H-indol-2-yl)-3-methylimidazo[1,2-a]pyridin-7-yl)methanone (4),
(R)-(3-aminopiperidin-1-yl)(2-(1-ethyl-3-phenyl-1H-indol-2-yl)-3-methylimidazo[1,2-a]pyridin-7-yl)methanone (5),
(R)-(3-aminopyrrolidin-1-yl)(2-(1-ethyl-3-phenyl-1H-indol-2-yl)-3-methylimidazo[1,2-a]pyridin-7-yl)methanone (6),
(R)-(3-aminopyrrolidin-1-yl)(2-(1-ethyl-1H-indol-2-yl)-3-methylimidazo[1,2-a]pyridin-7-yl)methanone (7),
(R)-(3-aminopyrrolidin-1-yl)(2-(1-(cyclopropylmethyl)-1H-indol-2-yl)-3-methylimidazo[1,2-a]pyridin-7-yl)methanone (8),
(2-(1-(cyclopropylmethyl)-1H-indol-2-yl)-3-methylimidazo[1,2-a]pyridin-7-yl)(hexahydro-2H-pyrido[4,3-b][1,4]oxazin-6(5H)-yl)methanone (9),
(R)-(3-aminopiperidin-1-yl)(3-methyl-2-(1-(pyridin-4-ylmethyl)-1H-indol-2-yl)imidazo[1,2-a]pyridin-7-yl)methanone (10),
(R)-(3-aminopiperidin-1-yl)(3-methyl-2-(1-((tetrahydro-2H-pyran-4-yl)methyl)-1H-indol-2-yl)imidazo[1,2-a]pyridin-7-yl)methanone (11),
(R)-(3-aminopiperidin-1-yl)(2-(1-(4-methoxybenzyl)-1H-indol-2-yl)-3-methylimidazo[1,2-a]pyridin-7-yl)methanone (12),
(R)-(3-aminopiperidin-1-yl)(2-(1-(4-fluorobenzyl)-1H-indol-2-yl)-3-methylimidazo[1,2-a]pyridin-7-yl)methanone (13),
(R)-4-((2-(7-(3-aminopiperidine-1-carbonyl)-3-methylimidazo[1,2-a]pyridin-2-yl)-1H-indol-1-yl)methyl)benzonitrile (14),
(R)-(3-aminopiperidin-1-yl)(3-methyl-2-(1-(pyridin-3-ylmethyl)-1H-indol-2-yl)imidazo[1,2-a]pyridin-7-yl)methanone (15),
(R)-(3-aminopiperidin-1-yl)(3-methyl-2-(1-(pyridin-2-ylmethyl)-1H-indol-2-yl)imidazo[1,2-a]pyridin-7-yl)methanone (16),
(R)-(3-aminopiperidin-1-yl)(3-methyl-2-(1-(2,2,2-trifluoroethyl)-1H-indol-2-yl)imidazo[1,2-a]pyridin-7-yl)methanone (17),
(R)-4-((2-(7-(3-aminopiperidine-1-carbonyl)-3-methylimidazo[1,2-a]pyridin-2-yl)-1H-indol-1-yl)methyl)-1-methylpyridin-2(1H)-one (18),
(R)-(3-aminopiperidin-1-yl)(2-(3-ethylbenzo[b]thiophen-2-yl)-3-methylimidazo[1,2-a]pyridin-7-yl)methanone (19),
(R)-(3-aminopiperidin-1-yl)(2-(1-(4-chlorobenzyl)-1H-indol-2-yl)-3-methylimidazo[1,2-a]pyridin-7-yl)methanone (20),
(R)-(3-aminopiperidin-1-yl)(2-(1-(2-fluorobenzyl)-1H-indol-2-yl)-3-methylimidazo[1,2-a]pyridin-7-yl)methanone (21),
(R)-(3-aminopiperidin-1-yl)(2-(1-ethyl-5-phenyl-1H-pyrrol-2-yl)-3-methylimidazo[1,2-a]pyridin-7-yl)methanone (22),
(R)-(3-aminopiperidin-1-yl)(2-(1-(cyclopropylmethyl)-6-methoxy-1H-indol-2-yl)-3-methylimidazo[1,2-a]pyridin-7-yl)methanone (23),
(R)-(3-aminopiperidin-1-yl)(2-(1-ethyl-1H-pyrrolo[2,3-b]pyridin-2-yl)-3-methylimidazo[1,2-a]pyridin-7-yl)methanone (24),
(R)-(3-aminopiperidin-1-yl)(2-(1-(cyclopropylmethyl)-1H-indol-2-yl)-3,5-dimethylimidazo[1,2-a]pyridin-7-yl)methanone (25),
(R)-(3-aminopiperidin-1-yl)(2-(1-benzyl-1H-indol-2-yl)-3,5-dimethylimidazo[1,2-a]pyridin-7-yl)methanone (26),
(R)-(3-aminopiperidin-1-yl)(3,5-dimethyl-2-(1-(pyridin-2-ylmethyl)-1H-indol-2-yl)imidazo[1,2-a]pyridin-7-yl)methanone (27),
(R)-(3-aminopiperidin-1-yl)(2-(1-(cyclopropylmethyl)-6-fluoro-1H-indol-2-yl)-3-methylimidazo[1,2-a]pyridin-7-yl)methanone (28),
(R)-(3-aminopiperidin-1-yl)(2-(1-(cyclopropylmethyl)-5-fluoro-1H-indol-2-yl)-3-methylimidazo[1,2-a]pyridin-7-yl)methanone (29),
(R)-(3-aminopiperidin-1-yl)(2-(1-(cyclopropylmethyl)-7-methyl-1H-indol-2-yl)-3-methylimidazo[1,2-a]pyridin-7-yl)methanone (30),
(R)-(3-aminopiperidin-1-yl)(2-(2-ethylphenyl)-3-methylimidazo[1,2-a]pyridin-7-yl)methanone (31),
(R)-(3-aminopiperidin-1-yl)(2-(1-(cyclopropylmethyl)-1H-indol-2-yl)-3-phenylimidazo[1,2-a]pyridin-7-yl)methanone (32),
(R)-(3-aminopiperidin-1-yl)(3-cyclopropyl-2-(1-(cyclopropylmethyl)-1H-indol-2-yl)imidazo[1,2-a]pyridin-7-yl)methanone (33),
(R)-(3-aminopiperidin-1-yl)(2-(1-(cyclopropylmethyl)-1H-indol-2-yl)-3-(methoxymethyl)imidazo[1,2-a]pyridin-7-yl)methanone (34),
(R)-(3-aminopiperidin-1-yl)(2-(1-(3-fluorobenzyl)-1H-indol-2-yl)-3-methylimidazo[1,2-a]pyridin-7-yl)methanone (35),
(R)-(3-aminopiperidin-1-yl)(3-methyl-2-(1-(thiophen-3-ylmethyl)-1H-indol-2-yl)imidazo[1,2-a]pyridin-7-yl)methanone (36),
(R)-(3-aminopiperidin-1-yl)(2-(1-(furan-3-ylmethyl)-1H-indol-2-yl)-3-methylimidazo[1,2-a]pyridin-7-yl)methanone (37),
(R)-3-aminopiperidin-1-yl)(2-(1-(1-(4-fluorophenyl)ethyl)-1H-indol-2-yl)-3-methylimidazo[1,2-a]pyridin-7-yl)methanone (38),
(R)-(3-aminopiperidin-1-yl)(2-(1-ethyl-5-fluoro-1H-indol-2-yl)-3-methylimidazo[1,2-a]pyridin-7-yl)methanone (39),
(R)-(3-aminopiperidin-1-yl)(2-(1-(cyclopropylmethyl)-4-fluoro-1H-indol-2-yl)-3-methylimidazo[1,2-a]pyridin-7-yl)methanone (40),
(R)-(3-aminopiperidin-1-yl)(3-methyl-2-(1-((4-methylthiazol-2-yl)methyl)-1H-indol-2-yl)imidazo[1,2-a]pyridin-7-yl)methanone (41),
(R)-(3-aminopiperidin-1-yl)(2-(1-(3-methoxybenzyl)-1H-indol-2-yl)-3-methylimidazo[1,2-a]pyridin-7-yl)methanone (42),
(R)-(3-aminopiperidin-1-yl)(2-(5-bromo-1-(cyclopropylmethyl)-1H-indol-2-yl)-3-methylimidazo[1,2-a]pyridin-7-yl)methanone (43),
(R)-(3-aminopiperidin-1-yl)(2-(7-chloro-1-(cyclopropylmethyl)-1H-indol-2-yl)-3-methylimidazo[1,2-a]pyridin-7-yl)methanone (44),
(R)-(3-aminopiperidin-1-yl)(2-(1-(4-fluorobenzyl)-1H-indol-2-yl)-3-methylimidazo[1,2-a]pyridin-7-yl)methanethione (45),
(R)-(3-aminopiperidin-1-yl)(3-methyl-2-(1-methyl-1H-indol-2-yl)imidazo[1,2-a]pyridin-7-yl)methanone (46),
(R)-(3-aminopiperidin-1-yl)(2-(1-(cyclopropylmethyl)-7-methoxy-1H-indol-2-yl)-3-methylimidazo[1,2-a]pyridin-7-yl)methanone (47),
(R)-(3-aminopiperidin-1-yl)(2-(1-((2,4-dimethylthiazol-5-yl)methyl)-1H-indol-2-yl)-3-methylimidazo[1,2-a]pyridin-7-yl)methanone (48),
(R)-(3-aminopiperidin-1-yl)(3-methyl-2-(1-((2-methylthiazol-5-yl)methyl)-1H-indol-2-yl)imidazo[1,2-a]pyridin-7-yl)methanone (49),
(R)-(3-aminopiperidin-1-yl) (2-(1-(3-methoxybenzyl)-1H-indol-2-yl)-3-methylimidazo[1,2-a]pyridin-7-yl)methanone trifluoroacetic acid salt (50),
(R)-(3-aminopiperidin-1-yl)(2-(6-methoxy-1-(pyridin-3-ylmethyl)-1H-indol-2-yl)-3-methylimidazo[1,2-a]pyridin-7-yl)methanone (51),
(R)-(3-aminopiperidin-1-yl)(2-(1-(cyclopropylmethyl)-6-fluoro-1H-indol-2-yl)-3-methylimidazo[1,2-a]pyridin-7-yl)methanone trifluoroacetic acid salt (52),
(R)-(3-aminopiperidin-1-yl)(2-(5-fluoro-1-(3-methoxybenzyl)-1H-indol-2-yl)-3-methylimidazo[1,2-a]pyridin-7-yl)methanone (53),
(R)-(3-aminopiperidin-1-yl)(2-(6-fluoro-1-(4-fluorobenzyl)-1H-indol-2-yl)-3-methylimidazo[1,2-a]pyridin-7-yl)methanone (54),
(R)-(3-aminopiperidin-1-yl)(2-(5,6-difluoro-1-(3-methoxybenzyl)-1H-indol-2-yl)-3-methylimidazo[1,2-a]pyridin-7-yl)methanone (55),
(R,E)-4-(dimethylamino)-N-(1-(2-(1-(4-fluorobenzyl)-1H-indol-2-yl)-3-methylimidazo[1,2-a]pyridine-7-carbonyl)piperidin-3-yl)but-2-enamide (56),
(R)-(3-aminopiperidin-1-yl)(3-methyl-2-(1-(pyrazin-2-ylmethyl)-1H-indol-2-yl)imidazo[1,2-a]pyridin-7-yl)methanone (57),
(R)-(3-aminopiperidin-1-yl)(3-methyl-2-(1-(pyrimidin-5-ylmethyl)-1H-indol-2-yl)imidazo[1,2-a]pyridin-7-yl)methanone trifluoroacetic acid salt (58),
(R)-(3-aminopiperidin-1-yl)(3-methyl-2-(1-(pyridazin-3-ylmethyl)-1H-indol-2-yl)imidazo[1,2-a]pyridin-7-yl)methanone (59),
(R)-(3-aminopiperidin-1-yl)(2-(1-isobutyl-1H-indol-2-yl)-3-methylimidazo[1,2-a]pyridin-7-yl)methanone (60),
(R)-(3-aminopiperidin-1-yl)(2-(1-(cyclobutylmethyl)-1H-indol-2-yl)-3-methylimidazo[1,2-a]pyridin-7-yl)methanone (61),
(R)-(3-aminopiperidin-1-yl)(2-(1-((3-fluoropyridin-2-yl)methyl)-1H-indol-2-yl)-3-methylimidazo[1,2-a]pyridin-7-yl)methanone (62),
(R)-(3-aminopiperidin-1-yl)(2-(1-((5-methoxypyridin-2-yl)methyl)-1H-indol-2-yl)-3-methylimidazo[1,2-a]pyridin-7-yl)methanone (63),
(R)-(3-aminopiperidin-1-yl)(2-(1-(2-methoxyethyl)-1H-indol-2-yl)-3-methylimidazo[1,2-a]pyridin-7-yl)methanone (64),
(R)-(3-aminopiperidin-1-yl)(2-(1-(2-hydroxyethyl)-1H-indol-2-yl)-3-methylimidazo[1,2-a]pyridin-7-yl)methanone (65),
(R)-(3-aminopiperidin-1-yl)(2-(6-methoxy-1-(pyridin-4-ylmethyl)-1H-indol-2-yl)-3-methylimidazo[1,2-a]pyridin-7-yl)methanone (66),
(R)-(3-aminopiperidin-1-yl)(2-(1-ethyl-6-methoxy-1H-indol-2-yl)-3-methylimidazo[1,2-a]pyridin-7-yl)methanone (67),
(R)-(3-aminopiperidin-1-yl)(2-(6-fluoro-1-(pyridin-4-ylmethyl)-1H-indol-2-yl)-3-methylimidazo[1,2-a]pyridin-7-yl)methanone (68),
(R)-(3-aminopiperidin-1-yl)(2-(6-fluoro-1-(pyridin-3-ylmethyl)-1H-indol-2-yl)-3-methylimidazo[1,2-a]pyridin-7-yl)methanone (69),
(R)-(3-aminopiperidin-1-yl)(2-(6-fluoro-1-(3-methoxybenzyl)-1H-indol-2-yl)-3-methylimidazo[1,2-a]pyridin-7-yl)methanone (70),
(R)-(3-aminopiperidin-1-yl)(2-(6-fluoro-1-(4-methoxybenzyl)-1H-indol-2-yl)-3-methylimidazo[1,2-a]pyridin-7-yl)methanone (71),
(R)-(3-aminopiperidin-1-yl)(2-(1-ethyl-6-fluoro-1H-indol-2-yl)-3-methylimidazo[1,2-a]pyridin-7-yl)methanone (72),
(R)-(3-aminopiperidin-1-yl)(2-(6-fluoro-1-isobutyl-1H-indol-2-yl)-3-methylimidazo[1,2-a]pyridin-7-yl)methanone (73),
(R)-(3-aminopiperidin-1-yl)(2-(5-fluoro-1-(pyridin-4-ylmethyl)-1H-indol-2-yl)-3-methylimidazo[1,2-a]pyridin-7-yl)methanone (74),
(R)-(3-aminopiperidin-1-yl)(2-(5-fluoro-1-(4-methoxybenzyl)-1H-indol-2-yl)-3-methylimidazo[1,2-a]pyridin-7-yl)methanone (75),
(R)-(3-aminopiperidin-1-yl)(2-(5-fluoro-1-(4-fluorobenzyl)-1H-indol-2-yl)-3-methylimidazo[1,2-a]pyridin-7-yl)methanone (76),
(R)-(3-aminopiperidin-1-yl)(2-(7-chloro-1-(4-fluorobenzyl)-1H-indol-2-yl)-3-methylimidazo[1,2-a]pyridin-7-yl)methanone (77),
(R)-(3-aminopiperidin-1-yl)(2-(1-(2,2-difluoroethyl)-1H-indol-2-yl)-3-methylimidazo[1,2-a]pyridin-7-yl)methanone trifluoroacetic acid salt (78),
(R)-(3-aminopiperidin-1-yl)(2-(1-((5-fluoropyridin-2-yl)methyl)-1H-indol-2-yl)-3-methylimidazo[1,2-a]pyridin-7-yl)methanone (79),
(R)-(3-aminopiperidin-1-yl)(2-(1-(4-fluorobenzyl)-6-methoxy-1H-indol-2-yl)-3-methylimidazo[1,2-a]pyridin-7-yl)methanone (80),
(R)-(3-aminopiperidin-1-yl)(2-(1-(4-(hydroxymethyl)benzyl)-1H-indol-2-yl)-3-methylimidazo[1,2-a]pyridin-7-yl)methanone (81),
(R)-(3-aminopiperidin-1-yl)(2-(1-isobutyl-6-methoxy-1H-indol-2-yl)-3-methylimidazo[1,2-a]pyridin-7-yl)methanone (82),
(R)-(3-aminopiperidin-1-yl)(2-(1-(2,2-difluoroethyl)-6-methoxy-1H-indol-2-yl)-3-methylimidazo[1,2-a]pyridin-7-yl)methanone (83),
(R)-(3-aminopiperidin-1-yl)(2-(5-fluoro-1-isobutyl-1H-indol-2-yl)-3-methylimidazo[1,2-a]pyridin-7-yl)methanone (84),
(R)-(3-aminopiperidin-1-yl)(2-(1-(4-fluoro-3-methoxybenzyl)-1H-indol-2-yl)-3-methylimidazo[1,2-a]pyridin-7-yl)methanone (85).
(R)-(3-aminopiperidin-1-yl)(2-(1-(cyclopropylmethyl)-6-methoxy-1H-indol-2-yl)-3-(methoxymethyl)imidazo[1,2-a]pyridin-7-yl)methanone (86)
(R)-(3-aminopiperidin-1-yl)(2-(1-(cyclopropylmethyl)-1H-indol-2-yl)-3,4-dihydro-5-oxa-1,2a-diazaacenaphthylen-7-yl)methanone (87),
(R)-(3-aminopyrrolidin-1-yl)(2-(1-(cyclopropylmethyl)-1H-indol-2-yl)-3,4-dihydro-5-oxa-1,2a-diazaacenaphthylen-7-yl)methanone (88),
(R)-(3-aminopiperidin-1-yl)(2-(1-ethyl-1H-indol-2-yl)-3,4-dihydro-5-oxa-1,2a-diazaacenaphthylen-7-yl)methanone (89),
(2-(aminomethyl)piperidin-1-yl)(2-(1-(cyclopropylmethyl)-1H-indol-2-yl)-3,4-dihydro-5-oxa-1,2a-diazaacenaphthylen-7-yl)methanone (90),
(R)-(3-aminopiperidin-1-yl)(2-(1-ethyl-1H-pyrrolo[2,3-b]pyridin-2-yl)-3,4-dihydro-5-oxa-1,2a-diazaacenaphthylen-7-yl)methanone (91),
(R)-(3-aminopiperidin-1-yl)(1-(1-(cyclopropylmethyl)-1H-indol-2-yl)-8,9-dihydro-7H-6-oxa-2,9a-diazabenzo[cd]azulen-4-yl)methanone (92),
(R)-(3-aminopyrrolidin-1-yl)(1-(1-(cyclopropylmethyl)-1H-indol-2-yl)-8,9-dihydro-7H-6-oxa-2,9a-diazabenzo[cd]azulen-4-yl)methanone (93),
(R)-(3-aminopiperidin-1-yl)(2-(1-(cyclopropylmethyl)-7-methyl-1H-indol-2-yl)-3,4-dihydro-5-oxa-1,2a-diazaacenaphthylen-7-yl)methanone (94),
(R)-(3-aminopiperidin-1-yl)(2-(1-(cyclopropylmethyl)-5-fluoro-1H-indol-2-yl)-3,4-dihydro-5-oxa-1,2a-diazaacenaphthylen-7-yl)methanone (95),
(R)-(3-aminopiperidin-1-yl)(2-(1-(pyridin-4-ylmethyl)-1H-indol-2-yl)-3,4-dihydro-5-oxa-1,2a-diazaacenaphthylen-7-yl)methanone (96),
(R)-(3-aminopiperidin-1-yl)(2-(1-(pyridin-2-ylmethyl)-1H-indol-2-yl)-3,4-dihydro-5-oxa-1,2a-diazaacenaphthylen-7-yl)methanone (97),
(R)-(3-aminopiperidin-1-yl)(2-(3-ethylbenzo[b]thiophen-2-yl)-3,4-dihydro-5-oxa-1,2a-diazaacenaphthylen-7-yl)methanone (98),
(R)-(3-aminopiperidin-1-yl)(2-(1-(4-chlorobenzyl)-1H-indol-2-yl)-3,4-dihydro-5-oxa-1,2a-diazaacenaphthylen-7-yl)methanone (99),
(R)-(3-aminopiperidin-1-yl)(2-(1-(2-fluorobenzyl)-1H-indol-2-yl)-3,4-dihydro-5-oxa-1,2a-diazaacenaphthylen-7-yl)methanone (100),
(R)-(3-aminopiperidin-1-yl)(2-(1-(4-fluorobenzyl)-1H-indol-2-yl)-3,4-dihydro-5-oxa-1,2a-diazaacenaphthylen-7-yl)methanone (101),
(R)-(3-aminopiperidin-1-yl)(2-(1-(pyridin-3-ylmethyl)-1H-indol-2-yl)-3,4-dihydro-5-oxa-1,2a-diazaacenaphthylen-7-yl)methanone (102),
(R)-(3-aminopiperidin-1-yl)(2-(1-(cyclopropylmethyl)-5,6-dimethoxy-1H-indol-2-yl)-3,4-dihydro-5-oxa-1,2a-diazaacenaphthylen-7-yl)methanone (103),
(R)-(3-aminopiperidin-1-yl)(2-(1-benzyl-1H-indol-2-yl)-3,4-dihydro-5-oxa-1,2a-diazaacenaphthylen-7-yl)methanone (104),
(R)-(3-aminopiperidin-1-yl)(2-(1-(4-methoxybenzyl)-1H-indol-2-yl)-3,4-dihydro-5-oxa-1,2a-diazaacenaphthylen-7-yl)methanone (105),
(R)-(3-aminopiperidin-1-yl)(2-(1-(2-methoxyethyl)-1H-indol-2-yl)-3,4-dihydro-5-oxa-1,2a-diazaacenaphthylen-7-yl)methanone (106),
(R)-(3-aminopiperidin-1-yl)(2-(6-methoxy-1-(2-methoxyethyl)-1H-indol-2-yl)-3,4-dihydro-5-oxa-1,2a-diazaacenaphthylen-7-yl)methanone (107),
(R)-(3-aminopiperidin-1-yl)(2-(1-(2-hydroxyethyl)-1H-indol-2-yl)-3,4-dihydro-5-oxa-1,2a-diazaacenaphthylen-7-yl)methanone (108),
(R)-(3-aminopiperidin-1-yl)(2-(1-(cyclopropylmethyl)-6-methoxy-1H-indol-2-yl)-3,4-dihydro-5-oxa-1,2a-diazaacenaphthylen-7-yl)methanone (109),
(R)-(3-aminopiperidin-1-yl)(2-(1-((3-fluoropyridin-4-yl)methyl)-1H-indol-2-yl)-3,4-dihydro-5-oxa-1,2a-diazaacenaphthylen-7-yl)methanone (110),
(R)-(3-aminopiperidin-1-yl)(2-(1-(pyrazin-2-ylmethyl)-1H-indol-2-yl)-3,4-dihydro-5-oxa-1,2a-diazaacenaphthylen-7-yl)methanone (111),
(R)-(3-aminopiperidin-1-yl)(2-(1-((3-fluoropyridin-2-yl)methyl)-1H-indol-2-yl)-3,4-dihydro-5-oxa-1,2a-diazaacenaphthylen-7-yl)methanone (112),
(R)-(3-aminopiperidin-1-yl)(2-(1-(pyrimidin-2-ylmethyl)-1H-indol-2-yl)-3,4-dihydro-5-oxa-1,2a-diazaacenaphthylen-7-yl)methanone (113),
(R)-(3-aminopiperidin-1-yl)(2-(1-(cyclopropylmethyl)-6-fluoro-1H-indol-2-yl)-3,4-dihydro-5-oxa-1,2a-diazaacenaphthylen-7-yl)methanone (114),
(R)-(3-aminopiperidin-1-yl)(2-(1-(pyrimidin-5-ylmethyl)-1H-indol-2-yl)-3,4-dihydro-5-oxa-1,2a-diazaacenaphthylen-7-yl)methanone (115),
(R)-(3-aminopiperidin-1-yl)(2-(1-(pyridazin-3-ylmethyl)-1H-indol-2-yl)-3,4-dihydro-5-oxa-1,2a-diazaacenaphthylen-7-yl)methanone (116),
(R)-(3-aminopiperidin-1-yl)(2-(1-isobutyl-1H-indol-2-yl)-3,4-dihydro-5-oxa-1,2a-diazaacenaphthylen-7-yl)methanone (117),
(R)-(3-aminopiperidin-1-yl)(2-(1-(cyclopropylmethyl)-5,6-difluoro-1H-indol-2-yl)-3,4-dihydro-5-oxa-1,2a-diazaacenaphthylen-7-yl)methanone (118),
(R)-(3-aminopiperidin-1-yl)(2-(1-((3-fluoropyridin-2-yl)methyl)-6-methoxy-1H-indol-2-yl)-3,4-dihydro-5-oxa-1,2a-diazaacenaphthylen-7-yl)methanone (119),
(R)-(3-aminopiperidin-1-yl)(2-(7-chloro-1-((3-fluoropyridin-2-yl)methyl)-1H-indol-2-yl)-3,4-dihydro-5-oxa-1,2a-diazaacenaphthylen-7-yl)methanone (120),
(R)-(3-aminopiperidin-1-yl)(2-(6-fluoro-1-((3-fluoropyridin-2-yl)methyl)-1H-indol-2-yl)-3,4-dihydro-5-oxa-1,2a-diazaacenaphthylen-7-yl)methanone (121),
(R)-(3-aminopiperidin-1-yl)(2-(7-chloro-1-(2-methoxyethyl)-1H-indol-2-yl)-3,4-dihydro-5-oxa-1,2a-diazaacenaphthylen-7-yl)methanone (122),
(R)-(3-aminopiperidin-1-yl)(2-(1-(4-(hydroxymethyl)benzyl)-1H-indol-2-yl)-3,4-dihydro-5-oxa-1,2a-diazaacenaphthylen-7-yl)methanone trifluoroacetic acid salt (123),
(R,E)-N-(1-(2-(1-(cyclopropylmethyl)-1H-indol-2-yl)-3,4-dihydro-5-oxa-1,2a-diazaacenaphthylene-7-carbonyl)piperidin-3-yl)-4-(dimethylamino)but-2-enamide trifluoroacetic acid salt (124),
(R)-(3-aminopiperidin-1-yl)(2-(7-chloro-1-(4-methoxybenzyl)-1H-indol-2-yl)-3,4-dihydro-5-oxa-1,2a-diazaacenaphthylen-7-yl)methanone (125),
(R)-(3-aminopiperidin-1-yl)(2-(1-((tetrahydro-2H-pyran-4-yl)methyl)-1H-indol-2-yl)-3,4-dihydro-5-oxa-1,2a-diazaacenaphthylen-7-yl)methanone (126),
(R)-(3-aminopiperidin-1-yl)(2-(1-(cyclobutylmethyl)-1H-indol-2-yl)-3,4-dihydro-5-oxa-1,2a-diazaacenaphthylen-7-yl)methanone (127),
(R)-2-(2-(7-(3-aminopiperidine-1-carbonyl)-3,4-dihydro-5-ox1,2adiazaacenaphthylen-2-yl)-1H-indol-1-yl)acetic acid (128),
(R)-(3-aminopiperidin-1-yl)(2-(1-(piperidin-4-ylmethyl)-1H-indol-2-yl)-3,4-dihydro-5-oxa-1,2a-diazaacenaphthylen-7-yl)methanone (129),
(R)-(3-aminopiperidin-1-yl)(2-(1-(oxetan-3-ylmethyl)-1H-indol-2-yl)-3,4-dihydro-5-oxa-1,2a-diazaacenaphthylen-7-yl)methanone (130),
(R)-(3-aminopiperidin-1-yl)(2-(1-((1-methylpiperidin-4-yl)methyl)-1H-indol-2-yl)-3,4-dihydro-5-oxa-1,2a-diazaacenaphthylen-7-yl)methanone (131),
(R)-(3-aminopiperidin-1-yl)(2-(5-fluoro-1-(4-methoxybenzyl)-1H-indol-2-yl)-3,4-dihydro-5-oxa-1,2a-diazaacenaphthylen-7-yl)methanone (132),
(R)-(3-aminopiperidin-1-yl)(2-(1-(2,2-difluoroethyl)-1H-indol-2-yl)-3,4-dihydro-5-oxa-1,2a-diazaacenaphthylen-7-yl)methanone (133),
(R)-(3-aminopiperidin-1-yl)(2-(5-fluoro-1-(2-methoxyethyl)-1H-indol-2-yl)-3,4-dihydro-5-oxa-1,2a-diazaacenaphthylen-7-yl)methanone (134),
(R)-(3-aminopiperidin-1-yl)(2-(6-fluoro-1-(4-fluorobenzyl)-1H-indol-2-yl)-3,4-dihydro-5-oxa-1,2a-diazaacenaphthylen-7-yl)methanone (135),
(R)-(3-aminopiperidin-1-yl)(2-(6-fluoro-1-(4-methoxybenzyl)-1H-indol-2-yl)-3,4-dihydro-5-oxa-1,2a-diazaacenaphthylen-7-yl)methanone (136),
(R)-(3-aminopiperidin-1-yl)(2-(1-(4-fluorobenzyl)-6-methoxy-1H-indol-2-yl)-3,4-dihydro-5-oxa-1,2a-diazaacenaphthylen-7-yl)methanone (137),
3-aminopiperidin-1-yl)(2-(6-fluoro-1-(2-methoxyethyl)-1H-indol-2-yl)-3,4-dihydro-5-oxa-1,2a-diazaacenaphthylen-7-yl)methanone (138),
(R)-(3-aminopiperidin-1-yl)(2-(7-chloro-1-(cyclobutylmethyl)-1H-indol-2-yl)-3,4-dihydro-5-oxa-1,2a-diazaacenaphthylen-7-yl)methanone (139),
(R)-(3-aminopiperidin-1-yl)(2-(5,6-difluoro-1-(2-methoxyethyl)-1H-indol-2-yl)-3,4-dihydro-5-oxa-1,2a-diazaacenaphthylen-7-yl)methanone (140),
(R)-(3-aminopiperidin-1-yl)(2-(7-chloro-1-isobutyl-1H-indol-2-yl)-3,4-dihydro-5-oxa-1,2a-diazaacenaphthylen-7-yl)methanone (141),
(R)-(3-aminopiperidin-1-yl)(2-(7-chloro-1-(2,2-difluoroethyl)-1H-indol-2-yl)-3,4-dihydro-5-oxa-1,2a-diazaacenaphthylen-7-yl)methanone (142),
(R)-(3-aminopiperidin-1-yl)(2-(7-chloro-1-(cyclopropylmethyl)-1H-indol-2-yl)-3,4-dihydro-5-oxa-1,2a-diazaacenaphthylen-7-yl)methanone (143),
(R,E)-(3-aminopiperidin-1-yl)(2-(1-(cyclopropylmethyl)-5-styryl-1H-indol-2-yl)-3,4-dihydro-5-oxa-1,2a-diazaacenaphthylen-7-yl)methanone (144),
(R)-(3-aminopiperidin-1-yl)(2-(1-((4-methylthiazol-2-yl)methyl)-1H-indol-2-yl)-3,4-dihydro-5-oxa-1,2a-diazaacenaphthylen-7-yl)methanone (145),
(R)-(3-aminopiperidin-1-yl)(2-(1-(cyclopropylmethyl)-5-methoxy-1H-indol-2-yl)-3,4-dihydro-5-oxa-1,2a-diazaacenaphthylen-7-yl)methanone (146),
(R)-(3-aminopiperidin-1-yl)(2-(1-(cyclopropylmethyl)-6-(hydroxymethyl)-1H-indol-2-yl)-3,4-dihydro-5-oxa-1,2a-diazaacenaphthylen-7-yl) methanone (147),
(R)-(3-aminopiperidin-1-yl)(2-(1-(cyclopropylmethyl)-1H-indol-2-yl)-3,3-dimethyl-3,4-dihydro-5-oxa-1,2a-diazaacenaphthylen-7-yl)methanone (148),
(R)-(3-aminopiperidin-1-yl)(2-(1-(cyclopropylmethyl)-1H-indol-2-yl)-5,6-dihydro-4H-imidazo[1,5,4-de]quinoxalin-8-yl)methanone (149),
(R)-(3-aminopiperidin-1-yl)(2-(1-(cyclopropylmethyl)-5-fluoro-1H-indol-2-yl)-5,6-dihydro-4H-imidazo[1,5,4-de]quinoxalin-8-yl)methanone (150),
(R)-(3-aminopiperidin-1-yl)(2-(1-(cyclopropylmethyl)-6-fluoro-1H-indol-2-yl)-5,6-dihydro-4H-imidazo[1,5,4-de]quinoxalin-8-yl)methanone (151),
(R)-(3-aminopiperidin-1-yl)(2-(1-(pyridin-3-ylmethyl)-1H-indol-2-yl)-5,6-dihydro-4H-imidazo[1,5,4-de]quinoxalin-8-yl)methanone (152),
(R)-(3-aminopiperidin-1-yl)(2-(5-bromo-1-(cyclopropylmethyl)-1H-indol-2-yl)-5,6-dihydro-4H-imidazo[1,5,4-de]quinoxalin-8-yl)methanone (153),
(R)-(3-aminopiperidin-1-yl)(2-(1-(cyclopropylmethyl)-5-(pyridin-3-yl)-1H-indol-2-yl)-5,6-dihydro-4H-imidazo[1,5,4-de]quinoxalin-8-yl)methanone (154),
(R)-(3-aminopiperidin-1-yl)(2-(1-(pyridin-2-ylmethyl)-1H-indol-2-yl)-5,6-dihydro-4H-imidazo[1,5,4-de]quinoxalin-8-yl)methanone (155),
(R)-(3-aminopiperidin-1-yl)(2-(1-(2-fluorobenzyl)-1H-indol-2-yl)-5,6-dihydro-4H-imidazo[1,5,4-de]quinoxalin-8-yl)methanone (156),
(R)-(3-aminopiperidin-1-yl)(2-(1-(pyridin-4-ylmethyl)-1H-indol-2-yl)-5,6-dihydro-4H-imidazo[1,5,4-de]quinoxalin-8-yl)methanone (157),
(R)-(3-aminopiperidin-1-yl)(2-(1-(cyclopropylmethyl)-1H-indol-2-yl)-6-methyl-5,6-dihydro-4H-imidazo[1,5,4-de]quinoxalin-8-yl)methanone (158),
(R)-(3-aminopiperidin-1-yl)(2-(1-(cyclopropylmethyl)-6-methoxy-1H-indol-2-yl)-5,6-dihydro-4H-imidazo[1,5,4-de]quinoxalin-8-yl)methanone (159),
(R)-(3-aminopiperidin-1-yl)(2-(1-(cyclopropylmethyl)-6-methoxy-1H-indol-2-yl)-6-methyl-5,6-dihydro-4H-imidazo[1,5,4-de]quinoxalin-8-yl)methanone (160),
(R)-(3-aminopiperidin-1-yl)(2-(1-(cyclopropylmethyl)-5-fluoro-1H-indol-2-yl)-6-methyl-5,6-dihydro-4H-imidazo[1,5,4-de]quinoxalin-8-yl)methanone (161),
(R)-(3-aminopiperidin-1-yl)(2-(1-(cyclopropylmethyl)-1H-indol-2-yl)-6-(methylsulfonyl)-5,6-dihydro-4H-imidazo[1,5,4-de]quinoxalin-8-yl)methanone (162),
(R)-(3-aminopiperidin-1-yl)(2-(1-(cyclopropylmethyl)-5,6-difluoro-1H-indol-2-yl)-5,6-dihydro-4H-imidazo[1,5,4-de]quinoxalin-8-yl)methanone (163),
(R)-(3-aminopiperidin-1-yl)(6-cyclopropyl-2-(1-(cyclopropylmethyl)-1H-indol-2-yl)-5,6-dihydro-4H-imidazo[1,5,4-de]quinoxalin-8-yl)methanone (164),
(R)-(3-aminopiperidin-1-yl)(2-(1-(cyclopropylmethyl)-1H-indol-2-yl)-6-(phenethylsulfonyl)-5,6-dihydro-4H-imidazo[1,5,4-de]quinoxalin-8-yl)methanone (165),
(R)-(3-aminopiperidin-1-yl)(2-(1-(cyclopropylmethyl)-4-fluoro-1H-indol-2-yl)-5,6-dihydro-4H-imidazo[1,5,4-de]quinoxalin-8-yl)methanone (166),
(R)-(3-aminopiperidin-1-yl)(6-((4-chlorophenyl)sulfonyl)-2-(1-(cyclopropylmethyl)-1H-indol-2-yl)-5,6-dihydro-4H-imidazo[1,5,4-de]quinoxalin-8-yl)methanone (167),
(R)-(3-aminopiperidin-1-yl)(2-(1-(cyclopropylmethyl)-1H-indol-2-yl)-6-(cyclopropylsulfonyl)-5,6-dihydro-4H-imidazo[1,5,4-de]quinoxalin-8-yl)methanone (168),
(R)-(3-aminopiperidin-1-yl)(2-(1-(cyclopropylmethyl)-1H-indol-2-yl)-6-((2-ethoxyethyl)sulfonyl)-5,6-dihydro-4H-imidazo[1,5,4-de]quinoxalin-8-yl)methanone (169),
(R)-(3-aminopiperidin-1-yl)(2-(1-(cyclopropylmethyl)-7-methyl-1H-indol-2-yl)-5,6-dihydro-4H-imidazo[1,5,4-de]quinoxalin-8-yl)methanone (170),
(R)-1-(8-(3-aminopiperidine-1-carbonyl)-2-(1-(cyclopropylmethyl)-1H-indol-2-yl)-4,5-dihydro-6H-imidazo[1,5,4-de]quinoxalin-6-yl)ethan-1-one (171),
(R)-(3-aminopiperidin-1-yl)(2-(1-(cyclopropylmethyl)-1H-indol-2-yl)-5,6-dihydro-4H-imidazo[1,5,4-de]quinoxalin-8-yl)methanethione (172),
(R)-(3-aminopiperidin-1-yl)(2-(7-chloro-1-(cyclopropylmethyl)-6-fluoro-1H-indol-2-yl)-3,4-dihydro-5-oxa-1,2a-diazaacenaphthylen-7-yl)methanone (173), and
(R)-(3-aminopiperidin-1-yl)(2-(7-chloro-1-(pyrimidin-5-ylmethyl)-1H-indol-2-yl)-3,4-dihydro-5-oxa-1,2a-diazaacenaphthylen-7-yl)methanone (174).

In an embodiment of the present disclosure, there is provided a compound or its polymorph, stereoisomer, prodrug, solvate, co-crystal, intermediate, pharmaceutically acceptable salt, metabolite or composition thereof for use in the treatment of a disease or disorder or condition ameliorated by inhibition of NETosis as described herein, wherein the composition is a pharmaceutical composition comprising the compound together with a pharmaceutically acceptable carrier, optionally in combination with one or more other pharmaceutical compositions.

In yet another embodiment of the present disclosure, there is provided a pharmaceutical composition as described herein, wherein the composition is in the form selected from a group consisting of a tablet, capsule, powder, syrup, solution, aerosol and suspension.

In an embodiment of the present disclosure, there is provided a compound as disclosed herein, or a pharmaceutically acceptable salt thereof as described herein, wherein the pharmaceutically acceptable salt selected derived from inorganic bases such as like Li, Na, K, Ca, Mg, Fe, Cu, Zn and Mn; salts of organic bases such as N, N′-diacetylethylenediamine, glucamine, triethylamine, choline, dicyclohexylamine, benzylamine, trialkylamine, thiamine, guanidine, diethanolamine, α-phenylethylamine, piperidine, morpholine, pyridine, hydroxyethylpyrrolidine, hydroxyethylpiperidine, ammonium, substituted ammonium salts, aluminum salts and the like. Salts also include amino acid salts such as glycine, alanine, cystine, cysteine, lysine, arginine, phenylalanine, and guanidine. Salts may include acid addition salts where appropriate which are sulphates, nitrates, phosphates, perchlorates, borates, hydrohalides, acetates, tartrates, maleates, citrates, succinates, palmoates, methanesulphonates, tosylates, benzoates, salicylates, hydroxynaphthoates, benzenesulfonates, ascorbates, glycerophosphates, ketoglutarates.

In an embodiment of the present disclosure, there is provided compound or a pharmaceutically acceptable salt thereof for use in the manufacture of a medicament for inhibiting one or more PADs in a cell.

In an embodiment of the present disclosure, there is provided a method for inhibiting one or more PAD family in a cell with an effective amount of the compounds as disclosed herein or a pharmaceutically acceptable salt thereof together with a pharmaceutically acceptable carrier, optionally in combination with one or more other pharmaceutical compositions.

In an embodiment of the present disclosure, there is provided a method for the treatment of a condition mediated by one or more PADs, comprising administering to a subject suffering from a condition mediated by one or more PAD family, a therapeutically effective amount of the compounds as disclosed herein or a pharmaceutically acceptable salt thereof together with a pharmaceutically acceptable carrier, optionally in combination with one or more other pharmaceutical compositions.

In an embodiment of the present disclosure, there is provided a method for the treatment of a condition mediated by excessive NET formation which is mediated by PAD4 enzyme, comprising a therapeutically effective amount of the compounds as disclosed herein or a pharmaceutically acceptable salt thereof together with a pharmaceutically acceptable carrier, optionally in combination with one or more other pharmaceutical compositions.

In an embodiment of the present disclosure, there is provided a method for the treatment of a condition mediated by excessive NET formation and inhibition of the NET using of compounds as disclosed herein or a pharmaceutically acceptable salt thereof together with a pharmaceutically acceptable carrier, optionally in combination with one or more other pharmaceutical compositions.

In an embodiment of the present disclosure, there is provided a method for the treatment and/or prevention of PAD mediated disorder or disorders associated with PAD activity, comprising administering to a subject suffering from PAD mediated disorder or disorders associated with PAD activity a therapeutically effective amount of the compounds as disclosed herein or a pharmaceutically acceptable salt thereof together with a pharmaceutically acceptable carrier, optionally in combination with one or more other pharmaceutical compositions.

In an embodiment of the present disclosure, there is provided a method for the treatment and/or prevention of PAD mediated disorder or disorders associated with PAD, is selected from a group consisting of thrombosis, microthrombosis, idiopathic pulmonary fibrosis, ARDS, anti neutrophil cytoplasmic antibody (ANCA) associated vasculitis (AAV), and hidradenitis suppurativa (HS).

In an embodiment of the present disclosure, there is provided a method for the treatment of PAD mediated disorder, said method comprising administering a combination of compounds as disclosed herein or a pharmaceutically acceptable salts thereof together with a pharmaceutically acceptable carrier, optionally in combination with one or more other pharmaceutical compositions, and/or with other clinically relevant agents or biological agents to a subject in need thereof.

In an embodiment of the present disclosure, there is provided a method comprising administering a combination of the compound as disclosed herein or the pharmaceutical composition with other clinically relevant agents or biological agents to a subject in need thereof.

In an embodiment of the present disclosure, there is provided a method for the treatment of COVID-19 (corona virus disease of 2019) mediated disorder, said method comprising administering a combination of compounds as disclosed herein or a pharmaceutically acceptable salts thereof together with a pharmaceutically acceptable carrier, optionally in combination with one or more other pharmaceutical compositions, and/or with other clinically relevant agents or biological agents to a subject in need thereof.

In an embodiment of the present disclosure, there is provided a method for the treatment and/or prevention of PAD mediated disorder is selected from a group consisting of thrombosis, microthrombosis, idiopathic pulmonary fibrosis, ARDS, cystic fibrosis, anti neutrophil cytoplasmic antibody (ANCA) associated vasculitis (AAV), hidradenitis suppurativa (HS) and pulmonary inflammation.

In an embodiment of the present disclosure, there is provided a method for the treatment and/or prevention of NET formation mediated disorder is selected from a group consisting of thrombosis, microthrombosis, idiopathic pulmonary fibrosis, ARDS, cystic fibrosis, anti neutrophil cytoplasmic antibody (ANCA) associated vasculitis (AAV), hidradenitis suppurativa (HS) and pulmonary inflammation.

In an embodiment of the present disclosure, there is provided a method for the treatment and/or prevention of PAD mediated disorder is selected from a group consisting of thrombosis and microthrombosis,

In further embodiment of the present disclosure, there is provided use of compound as disclosed herein in the treatment of a disease or disorder or condition, wherein the disease or disorder or condition is selected from cancer, cancer metastasis, immune disorder, immunodeficiency disorder, inflammatory disorder and transplant rejection.

In yet another embodiment of the present disclosure, there is provided use of compound as disclosed herein in the treatment of a disease or disorder or condition, wherein the disease or disorder or condition is cancer.

In an embodiment of the present disclosure, there is provided a compound as disclosed herein or its polymorph, stereoisomer, prodrug, solvate, co-crystal, intermediate, pharmaceutically acceptable salt, metabolite or composition thereof for use in the treatment of a disease or disorder or condition ameliorated by inhibition of NETosis as described herein, wherein the disease or disorder or condition is selected from acute respiratory distress syndrome (ARDS), COVID-19 (corona virus disease of 2019) related lung infection, multi-organ failure or multi-organ dysfunction syndrome from ARDS, anti neutrophil cytoplasmic antibody (ANCA) associated vasculitis (AAV), cancer, cancer metastasis, immune disorder, immunodeficiency disorder, inflammatory disorder, transplant rejection, arthritis, rheumatoid arthritis (RA), hidradenitis suppurativa (HS), diabetes, thrombosis, stroke, chronic obstructive pulmonary disease (COPD), cystic fibrosis, pulmonary disease, inflammatory bowel disease (IBS), Crohn's disease, ulcerative colitis, indeterminate colitis or Alzheimer's Disease.

In further embodiment of the present disclosure, there is provided a compound as disclosed herein or its polymorph, stereoisomer, prodrug, solvate, co-crystal, intermediate, pharmaceutically acceptable salt, metabolite or composition thereof for use in the treatment of a disease or disorder or condition ameliorated by inhibition of NETosis as described herein, wherein the disease or disorder or condition is selected from systemic inflammatory response syndrome, decreasing neutrophil extra cellular traps (NETs) release, acute lung injury (ALI), acute respiratory distress syndrome (ARDS), COVID-19 (corona virus disease of 2019) related lung infection, multi-organ failure or multi-organ dysfunction syndrome from ARDS, anti neutrophil cytoplasmic Antibody (ANCA) associated vasculitis (AAV), hidradenitis suppurativa (HS), sepsis, infections, or cytokine storms induced by drugs or any agent, ischemic or haemorrhagic stroke, ischemic or drug-induced haemorrhagic transformation in the brain, haemorrhagic encephalopathy, traumatic brain injury, anoxic brain injury, chronic kidney disease, diabetes, deep vein thrombosis, systemic microthrombosis, atherosclerotic thrombosis, thromboembolism, systemic lupus erythematosus (SLE), rheumatoid arthritis, COPD; cystic fibrosis, pulmonary disease, inflammatory bowel disease (IBS), Crohn's disease, ulcerative colitis, indeterminate colitis or Alzheimer's Disease.

In an embodiment of the present disclosure, there is provided a compound as disclosed herein or its polymorph, stereoisomer, prodrug, solvate, co-crystal, intermediate, pharmaceutically acceptable salt, metabolite or composition thereof for use in the treatment of a disease or disorder or condition ameliorated by inhibition of NETosis as described herein, wherein the disease or disorder or condition is cancer. In further embodiment of the present disclosure, there is provided a compound as disclosed herein or its polymorph, stereoisomer, prodrug, solvate, co-crystal, intermediate, pharmaceutically acceptable salt, metabolite or composition thereof for use in the treatment of a disease or disorder or condition ameliorated by inhibition of NETosis as described herein, wherein the disease or disorder or condition is lung cancer. In yet another embodiment of the present disclosure, there is provided a compound as disclosed herein or its polymorph, stereoisomer, prodrug, solvate, co-crystal, intermediate, pharmaceutically acceptable salt, metabolite or composition thereof for use in the treatment of a disease or disorder or condition ameliorated by inhibition of NETosis as described herein, wherein the disease or disorder or condition is breast cancer.

In an embodiment of the present disclosure, there is provided a compound as disclosed herein or its polymorph, stereoisomer, prodrug, solvate, co-crystal, intermediate, pharmaceutically acceptable salt, metabolite or composition thereof for use in the treatment of a disease or disorder or condition ameliorated by inhibition of NETosis as described herein, wherein the cancer is selected from breast cancer, prostate cancer, pancreatic cancer, gastric cancer, lung cancer, colon cancer, rectal cancer, esophagus cancer, duodenal cancer, tongue cancer, pharyngeal cancer, brain tumor, neurinoma, clear cell carcinoma, non-small cell lung cancer, small cell lung cancer, liver cancer, kidney cancer, bile duct cancer, uterine body cancer, cervical cancer, ovarian cancer, urinary bladder, skin cancer, hemangioma, malignant lymphoma, malignant melanoma, thyroid cancer, bone tumor, vascular fibroma, glioblastoma, sarcoma, neuroendocrine tumors, retinoblastoma, penile cancer, pediatric solid cancer, renal cell carcinoma, lymphoma, myeloma and leukemia (including, for example acute myelogenous leukemia (AML), chronic myelogenous leukemia (CML), chronic neutrophilic leukemia, chronic eosinophilic leukemia, chronic lymphocytic leukemia (CLL), acute lymphoblastic leukemia (ALL), hairy cell leukemia, cutaneous T-cell lymphoma (CTCL), multiple myeloma (MM), myeloproliferative neoplasms (MPN), a disease category that includes polycythemia vera (PV), essential thrombocythemia, essential thrombocytosis (ET) and myelofibrosis (MF), chronic myelogenous leukemia (CML), chronic neutrophilic leukemia (CNL), and chronic eosinophilic leukemia (CEL).

In another embodiment, there is provided a compound as disclosed herein or its polymorph, stereoisomer, prodrug, solvate, co-crystal, intermediate, pharmaceutically acceptable salt, metabolite or composition thereof for use in the treatment of a disease or disorder or condition ameliorated by inhibition of NETosis as described herein, wherein the disease or disorder or condition is cancer metastasis.

In further embodiment, there is provided a compound as disclosed herein or its polymorph, stereoisomer, prodrug, solvate, co-crystal, intermediate, pharmaceutically acceptable salt, metabolite or composition thereof for use in the treatment of a disease or disorder or condition ameliorated by inhibition of NETosis as described herein, wherein the cancer metastasis is liver cancer metastasis, lung cancer metastasis, and omentum cancer metastasis.

In yet another embodiment, there is provided a compound as disclosed herein or its polymorph, stereoisomer, prodrug, solvate, co-crystal, intermediate, pharmaceutically acceptable salt, metabolite or composition thereof for use in the treatment of a disease or disorder or condition ameliorated by inhibition of NETosis as described herein, wherein the disease or disorder or condition is cancer metastasis and the cancer metastasis is selected from liver cancer metastasis, lung cancer metastasis, and omentum cancer metastasis.

In yet another embodiment, there is provided a compound as disclosed herein or its polymorph, stereoisomer, prodrug, solvate, co-crystal, intermediate, pharmaceutically acceptable salt, metabolite or composition thereof for use in the treatment of a disease or disorder or condition ameliorated by inhibition of NETosis as described herein, wherein the cancer metastasis is liver cancer metastasis originating from colorectal cancer and pancreatic cancer, lung cancer metastasis originating from breast cancer, and omentum cancer metastasis originating from ovarian cancer.

In another embodiment of the present disclosure, there is provided a compound as disclosed herein or its polymorph, stereoisomer, prodrug, solvate, co-crystal, intermediate, pharmaceutically acceptable salt, metabolite, composition or the pharmaceutical composition comprising the compound of Formula (I) together with a pharmaceutically acceptable carrier in the treatment of a disease or disorder or condition ameliorated by inhibition of NETosis together with other clinically relevant cytotoxic agents or non-cytotoxic agents.

In another embodiment of the present disclosure, there is provided a compound as disclosed herein or its polymorph, stereoisomer, prodrug, solvate, co-crystal, intermediate, pharmaceutically acceptable salt, metabolite, composition or the pharmaceutical composition comprising the compound of Formula (I) together with a pharmaceutically acceptable carrier in the treatment of a disease or disorder or condition ameliorated by inhibition of NETosis together with other clinically relevant immune modulator agents or anti-inflammatory agents to a subject in need of thereof.

### EXAMPLES

The synthetic procedure and characterization of the compounds of the present disclosure have been exemplified in the patent WO 2019058393A1 and WO 20199907763A1.

The compound of the present disclosure is selected from the following list of compounds.

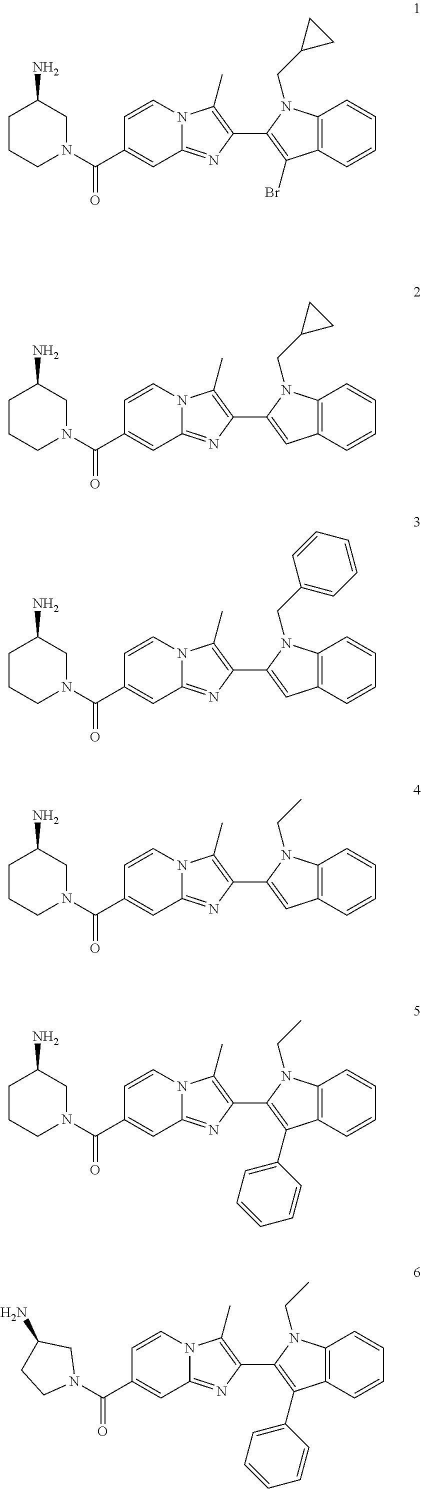 [SMILES: Cc1c(-c2c(Br)c3ccccc3n2CC2CC2)nc2cc(C(=O)N3CCC[C@@H](N)C3)ccn12 ; Cc1c(-c2cc3ccccc3n2CC2CC2)nc2cc(C(=O)N3CCC[C@@H](N)C3)ccn12 ; Cc1c(-c2cc3ccccc3n2Cc2ccccc2)nc2cc(C(=O)N3CCC[C@@H](N)C3)ccn12 ; CCn1c(-c2nc3cc(C(=O)N4CCC[C@@H](N)C4)ccn3c2C)cc2ccccc21 ; CCn1c(-c2nc3cc(C(=O)N4CCC[C@@H](N)C4)ccn3c2C)c(-c2ccccc2)c2ccccc21 ; CCn1c(-c2nc3cc(C(=O)N4CC[C@@H](N)C4)ccn3c2C)c(-c2ccccc2)c2ccccc21]

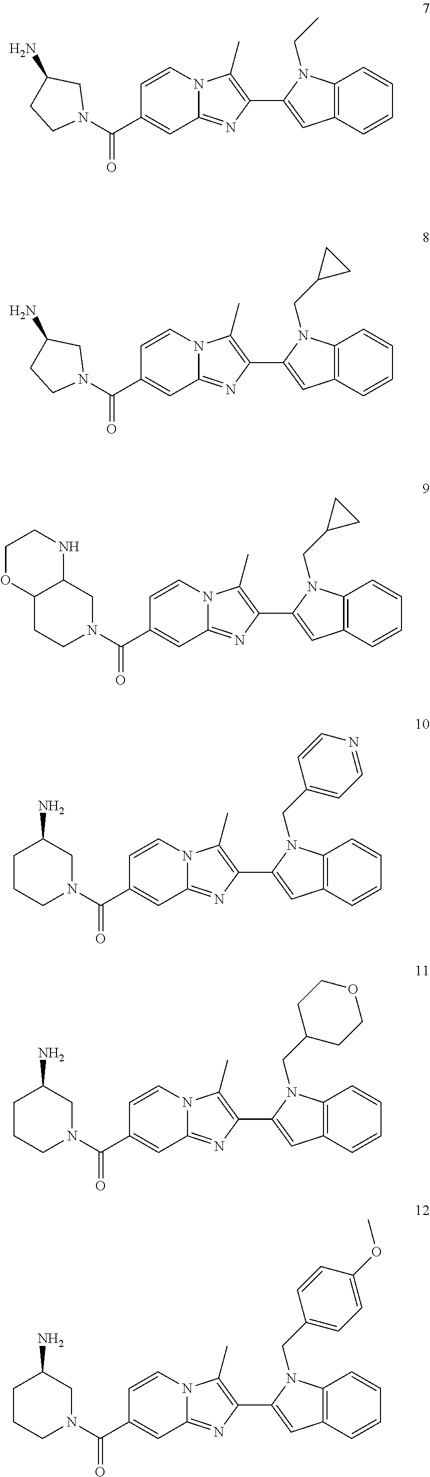 [SMILES: CCn1c(-c2nc3cc(C(=O)N4CC[C@@H](N)C4)ccn3c2C)cc2ccccc21 ; Cc1c(-c2cc3ccccc3n2CC2CC2)nc2cc(C(=O)N3CC[C@@H](N)C3)ccn12 ; Cc1c(-c2cc3ccccc3n2CC2CC2)nc2cc(C(=O)N3CCC4OCCNC4C3)ccn12 ; Cc1c(-c2cc3ccccc3n2Cc2ccncc2)nc2cc(C(=O)N3CCC[C@@H](N)C3)ccn12 ; Cc1c(-c2cc3ccccc3n2CC2CCOCC2)nc2cc(C(=O)N3CCC[C@@H](N)C3)ccn12 ; COc1ccc(Cn2c(-c3nc4cc(C(=O)N5CCC[C@@H](N)C5)ccn4c3C)cc3ccccc32)cc1]

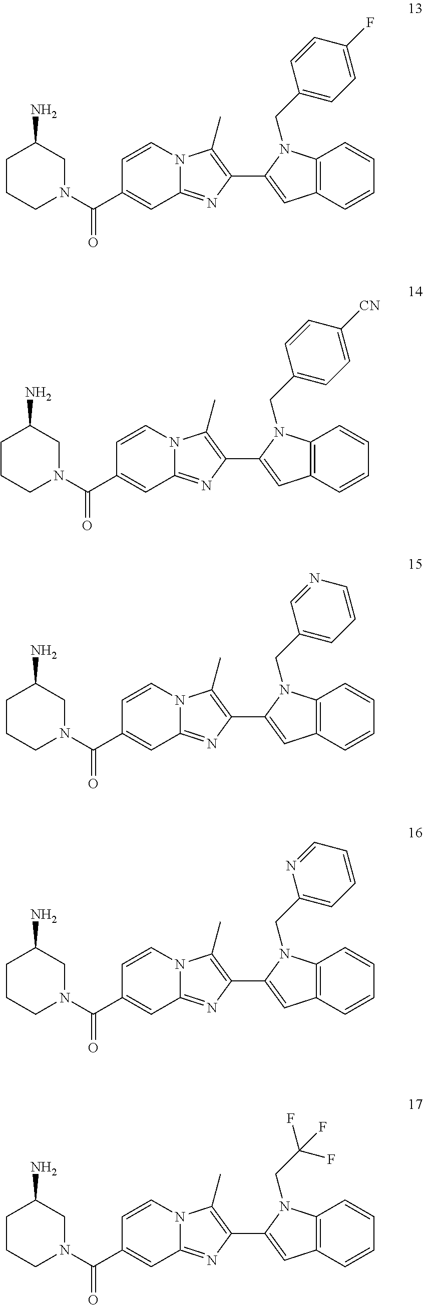 [SMILES: Cc1c(-c2cc3ccccc3n2Cc2ccc(F)cc2)nc2cc(C(=O)N3CCC[C@@H](N)C3)ccn12 ; Cc1c(-c2cc3ccccc3n2Cc2ccc(C#N)cc2)nc2cc(C(=O)N3CCC[C@@H](N)C3)ccn12 ; Cc1c(-c2cc3ccccc3n2Cc2cccnc2)nc2cc(C(=O)N3CCC[C@@H](N)C3)ccn12 ; Cc1c(-c2cc3ccccc3n2Cc2ccccn2)nc2cc(C(=O)N3CCC[C@@H](N)C3)ccn12 ; Cc1c(-c2cc3ccccc3n2CC(F)(F)F)nc2cc(C(=O)N3CCC[C@@H](N)C3)ccn12]

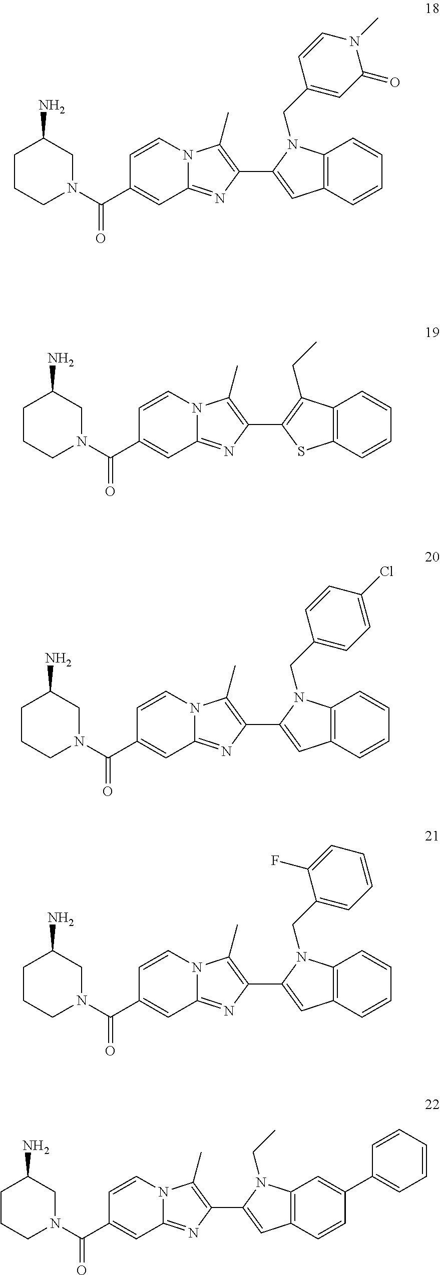 [SMILES: Cc1c(-c2cc3ccccc3n2Cc2ccn(C)c(=O)c2)nc2cc(C(=O)N3CCC[C@@H](N)C3)ccn12 ; CCc1c(-c2nc3cc(C(=O)N4CCC[C@@H](N)C4)ccn3c2C)sc2ccccc12 ; Cc1c(-c2cc3ccccc3n2Cc2ccc(Cl)cc2)nc2cc(C(=O)N3CCC[C@@H](N)C3)ccn12 ; Cc1c(-c2cc3ccccc3n2Cc2ccccc2F)nc2cc(C(=O)N3CCC[C@@H](N)C3)ccn12 ; CCn1c(-c2nc3cc(C(=O)N4CCC[C@@H](N)C4)ccn3c2C)cc2ccc(-c3ccccc3)cc21]

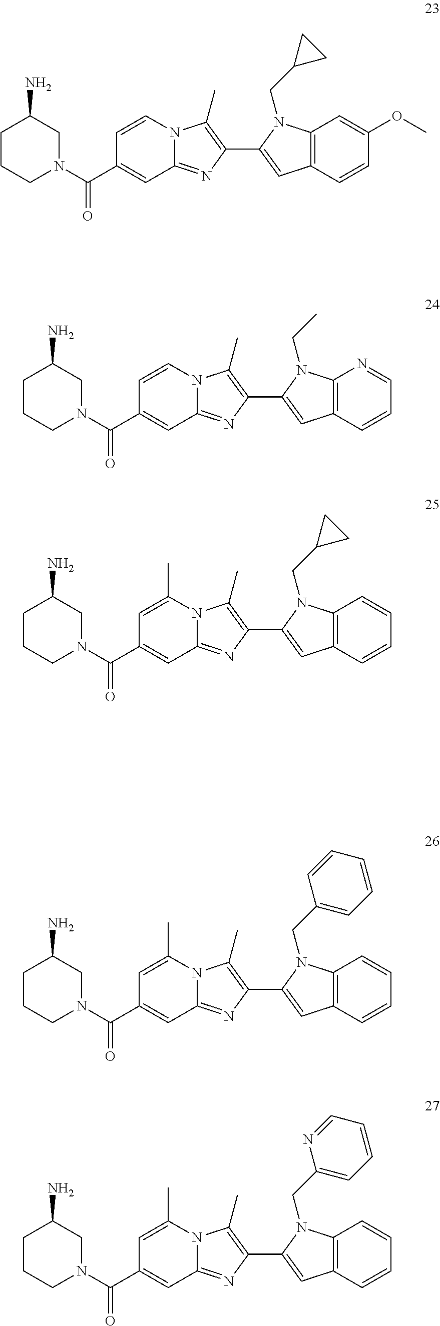 [SMILES: COc1ccc2cc(-c3nc4cc(C(=O)N5CCC[C@@H](N)C5)ccn4c3C)n(CC3CC3)c2c1 ; CCn1c(-c2nc3cc(C(=O)N4CCC[C@@H](N)C4)ccn3c2C)cc2cccnc21 ; Cc1cc(C(=O)N2CCC[C@@H](N)C2)cc2nc(-c3cc4ccccc4n3CC3CC3)c(C)n12 ; Cc1cc(C(=O)N2CCC[C@@H](N)C2)cc2nc(-c3cc4ccccc4n3Cc3ccccc3)c(C)n12 ; Cc1cc(C(=O)N2CCC[C@@H](N)C2)cc2nc(-c3cc4ccccc4n3Cc3ccccn3)c(C)n12]

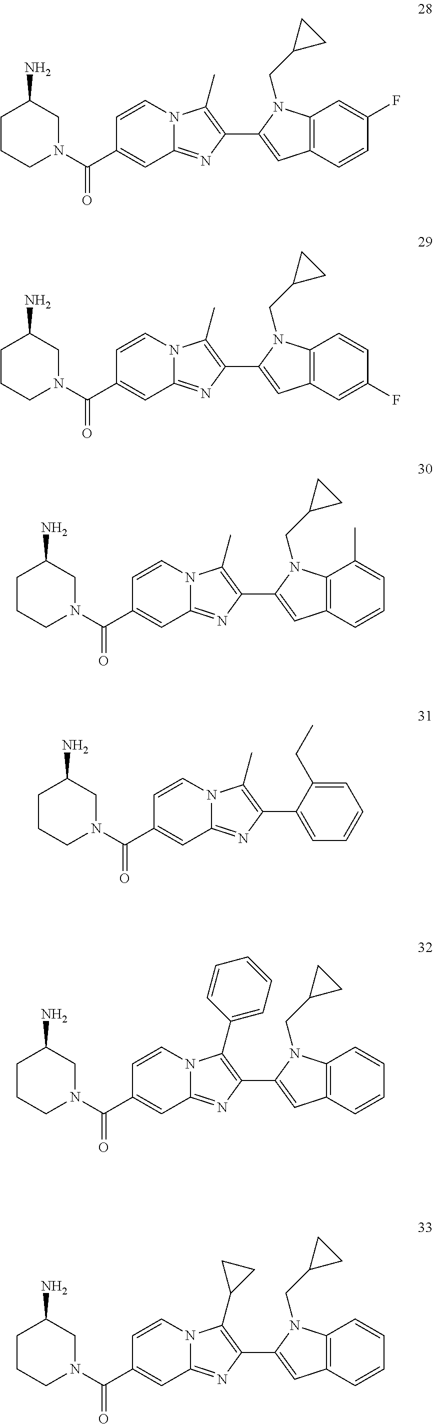 [SMILES: Cc1c(-c2cc3ccc(F)cc3n2CC2CC2)nc2cc(C(=O)N3CCC[C@@H](N)C3)ccn12 ; Cc1c(-c2cc3cc(F)ccc3n2CC2CC2)nc2cc(C(=O)N3CCC[C@@H](N)C3)ccn12 ; Cc1cccc2cc(-c3nc4cc(C(=O)N5CCC[C@@H](N)C5)ccn4c3C)n(CC3CC3)c12 ; CCc1ccccc1-c1nc2cc(C(=O)N3CCC[C@@H](N)C3)ccn2c1C ; N[C@@H]1CCCN(C(=O)c2ccn3c(-c4ccccc4)c(-c4cc5ccccc5n4CC4CC4)nc3c2)C1 ; N[C@@H]1CCCN(C(=O)c2ccn3c(C4CC4)c(-c4cc5ccccc5n4CC4CC4)nc3c2)C1]

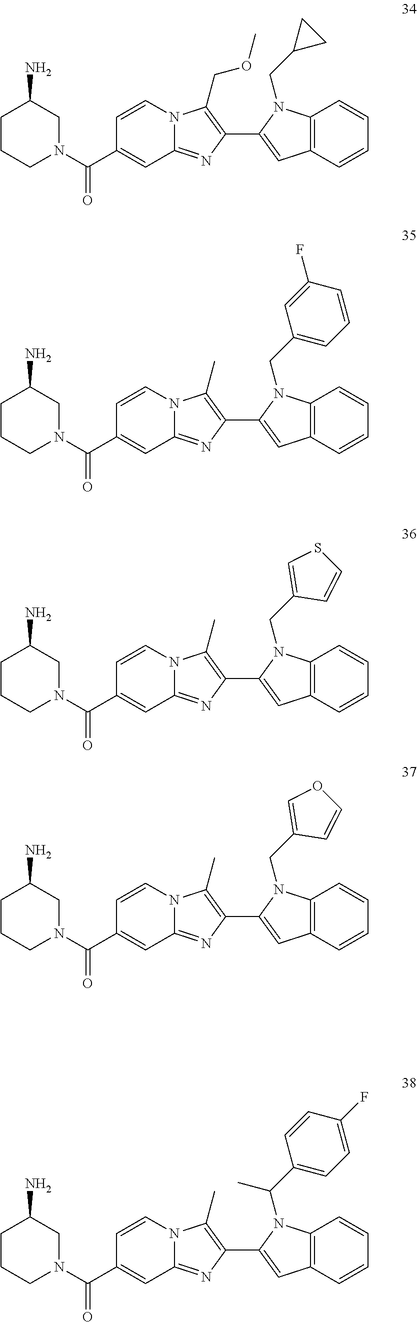 [SMILES: COCc1c(-c2cc3ccccc3n2CC2CC2)nc2cc(C(=O)N3CCC[C@@H](N)C3)ccn12 ; Cc1c(-c2cc3ccccc3n2Cc2cccc(F)c2)nc2cc(C(=O)N3CCC[C@@H](N)C3)ccn12 ; Cc1c(-c2cc3ccccc3n2Cc2ccsc2)nc2cc(C(=O)N3CCC[C@@H](N)C3)ccn12 ; Cc1c(-c2cc3ccccc3n2Cc2ccoc2)nc2cc(C(=O)N3CCC[C@@H](N)C3)ccn12 ; Cc1c(-c2cc3ccccc3n2C(C)c2ccc(F)cc2)nc2cc(C(=O)N3CCC[C@@H](N)C3)ccn12]

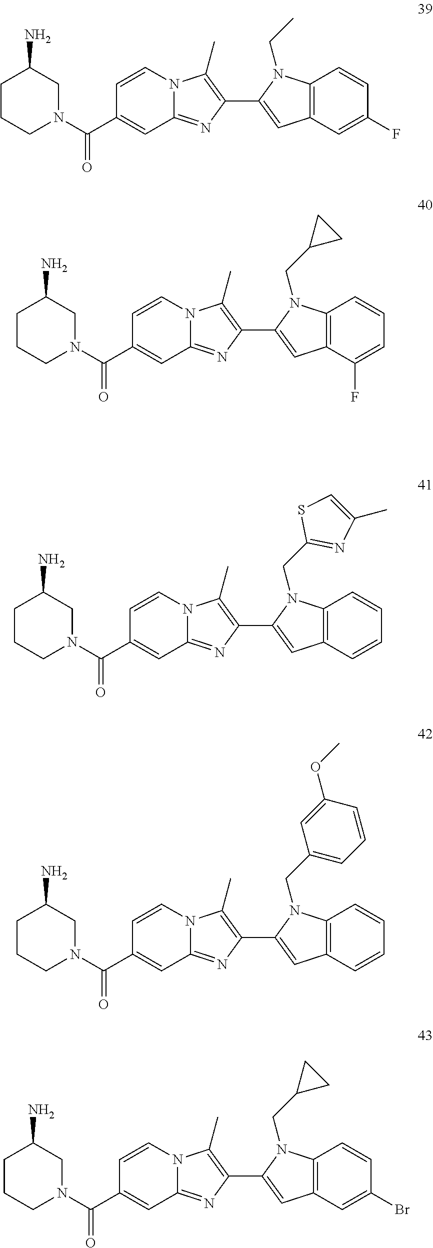 [SMILES: CCn1c(-c2nc3cc(C(=O)N4CCC[C@@H](N)C4)ccn3c2C)cc2cc(F)ccc21 ; Cc1c(-c2cc3c(F)cccc3n2CC2CC2)nc2cc(C(=O)N3CCC[C@@H](N)C3)ccn12 ; Cc1csc(Cn2c(-c3nc4cc(C(=O)N5CCC[C@@H](N)C5)ccn4c3C)cc3ccccc32)n1 ; COc1cccc(Cn2c(-c3nc4cc(C(=O)N5CCC[C@@H](N)C5)ccn4c3C)cc3ccccc32)c1 ; Cc1c(-c2cc3cc(Br)ccc3n2CC2CC2)nc2cc(C(=O)N3CCC[C@@H](N)C3)ccn12]

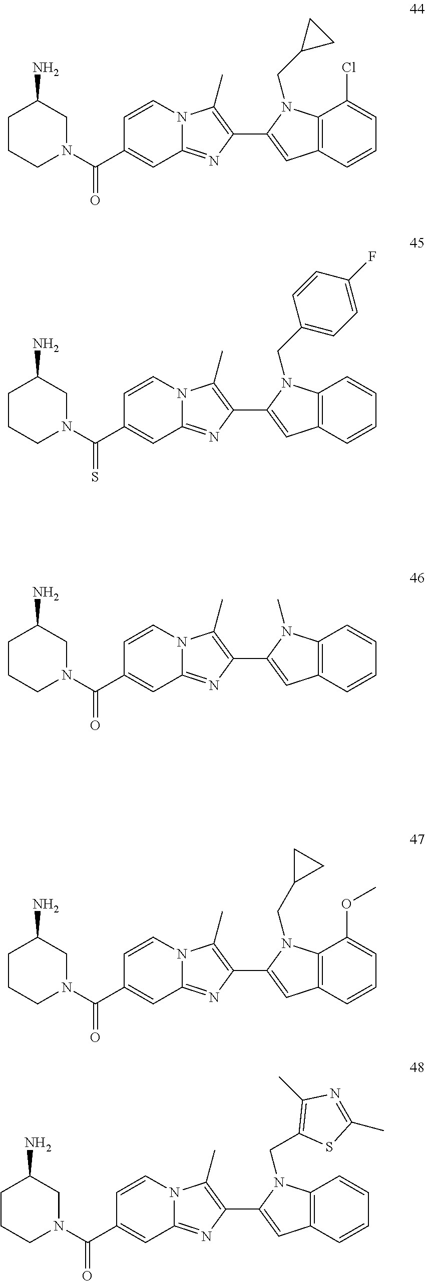 [SMILES: Cc1c(-c2cc3cccc(Cl)c3n2CC2CC2)nc2cc(C(=O)N3CCC[C@@H](N)C3)ccn12 ; Cc1c(-c2cc3ccccc3n2Cc2ccc(F)cc2)nc2cc(C(=S)N3CCC[C@@H](N)C3)ccn12 ; Cc1c(-c2cc3ccccc3n2C)nc2cc(C(=O)N3CCC[C@@H](N)C3)ccn12 ; COc1cccc2cc(-c3nc4cc(C(=O)N5CCC[C@@H](N)C5)ccn4c3C)n(CC3CC3)c12 ; Cc1nc(C)c(Cn2c(-c3nc4cc(C(=O)N5CCC[C@@H](N)C5)ccn4c3C)cc3ccccc32)s1]

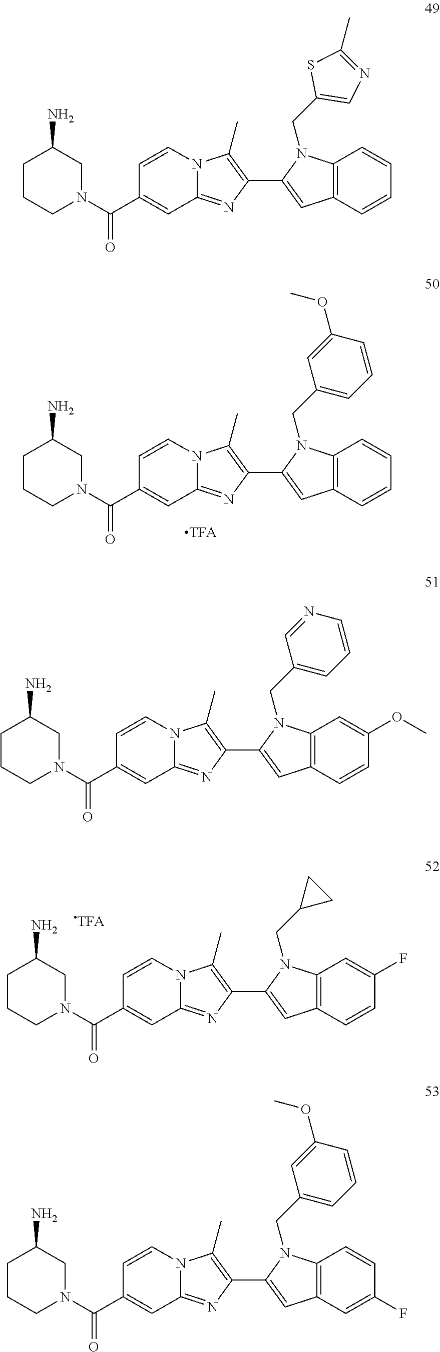 [SMILES: Cc1ncc(Cn2c(-c3nc4cc(C(=O)N5CCC[C@@H](N)C5)ccn4c3C)cc3ccccc32)s1 ; COc1cccc(Cn2c(-c3nc4cc(C(=O)N5CCC[C@@H](N)C5)ccn4c3C)cc3ccccc32)c1 ; COc1ccc2cc(-c3nc4cc(C(=O)N5CCC[C@@H](N)C5)ccn4c3C)n(Cc3cccnc3)c2c1 ; Cc1c(-c2cc3ccc(F)cc3n2CC2CC2)nc2cc(C(=O)N3CCC[C@@H](N)C3)ccn12 ; COc1cccc(Cn2c(-c3nc4cc(C(=O)N5CCC[C@@H](N)C5)ccn4c3C)cc3cc(F)ccc32)c1]

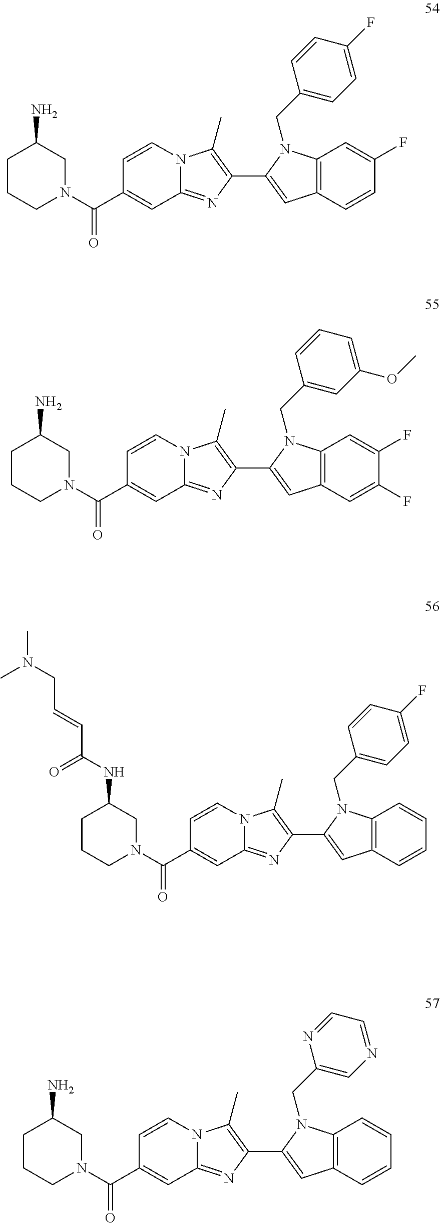

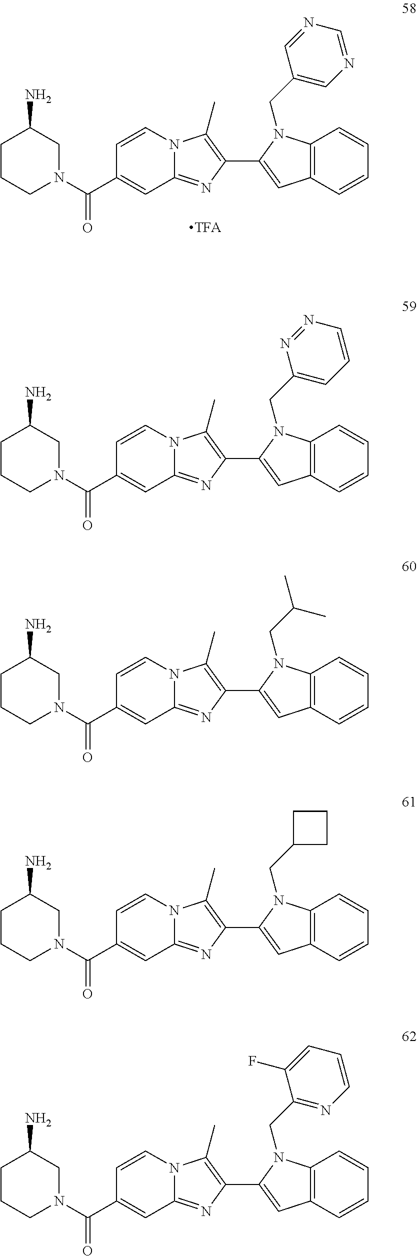 [SMILES: Cc1c(-c2cc3ccccc3n2Cc2cncnc2)nc2cc(C(=O)N3CCC[C@@H](N)C3)ccn12 ; Cc1c(-c2cc3ccccc3n2Cc2cccnn2)nc2cc(C(=O)N3CCC[C@@H](N)C3)ccn12 ; Cc1c(-c2cc3ccccc3n2CC(C)C)nc2cc(C(=O)N3CCC[C@@H](N)C3)ccn12 ; Cc1c(-c2cc3ccccc3n2CC2CCC2)nc2cc(C(=O)N3CCC[C@@H](N)C3)ccn12 ; Cc1c(-c2cc3ccccc3n2Cc2ncccc2F)nc2cc(C(=O)N3CCC[C@@H](N)C3)ccn12]

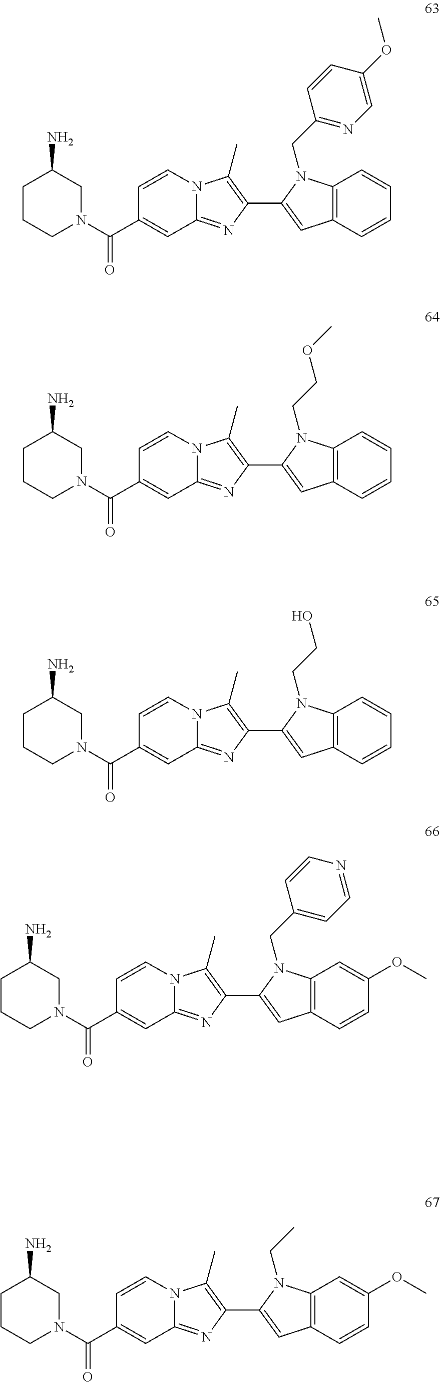 [SMILES: COc1ccc(Cn2c(-c3nc4cc(C(=O)N5CCC[C@@H](N)C5)ccn4c3C)cc3ccccc32)nc1 ; COCCn1c(-c2nc3cc(C(=O)N4CCC[C@@H](N)C4)ccn3c2C)cc2ccccc21 ; Cc1c(-c2cc3ccccc3n2CCO)nc2cc(C(=O)N3CCC[C@@H](N)C3)ccn12 ; COc1ccc2cc(-c3nc4cc(C(=O)N5CCC[C@@H](N)C5)ccn4c3C)n(Cc3ccncc3)c2c1 ; CCn1c(-c2nc3cc(C(=O)N4CCC[C@@H](N)C4)ccn3c2C)cc2ccc(OC)cc21]

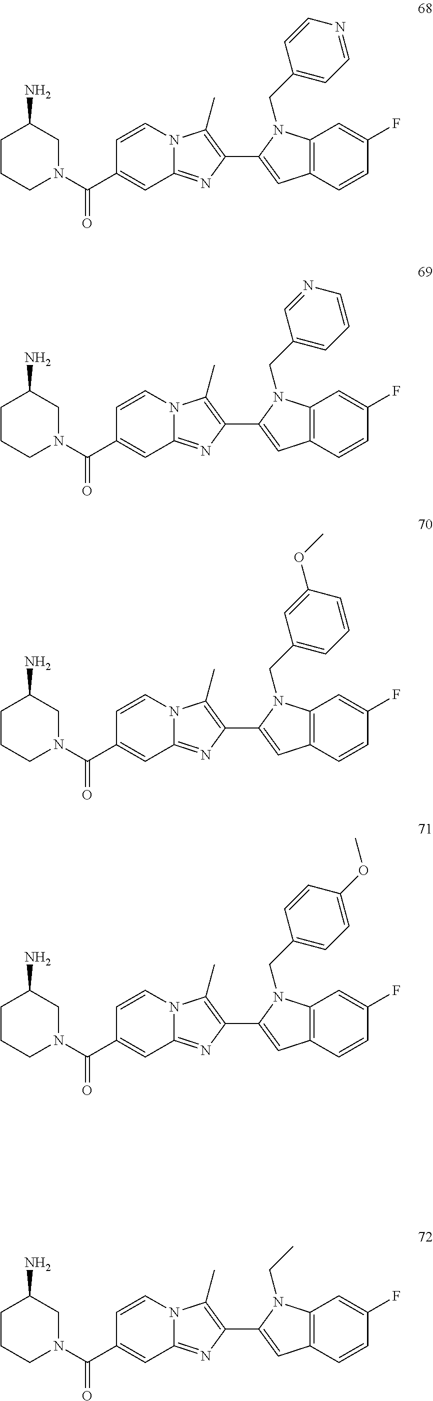 [SMILES: Cc1c(-c2cc3ccc(F)cc3n2Cc2ccncc2)nc2cc(C(=O)N3CCC[C@@H](N)C3)ccn12 ; Cc1c(-c2cc3ccc(F)cc3n2Cc2cccnc2)nc2cc(C(=O)N3CCC[C@@H](N)C3)ccn12 ; COc1cccc(Cn2c(-c3nc4cc(C(=O)N5CCC[C@@H](N)C5)ccn4c3C)cc3ccc(F)cc32)c1 ; COc1ccc(Cn2c(-c3nc4cc(C(=O)N5CCC[C@@H](N)C5)ccn4c3C)cc3ccc(F)cc32)cc1 ; CCn1c(-c2nc3cc(C(=O)N4CCC[C@@H](N)C4)ccn3c2C)cc2ccc(F)cc21]

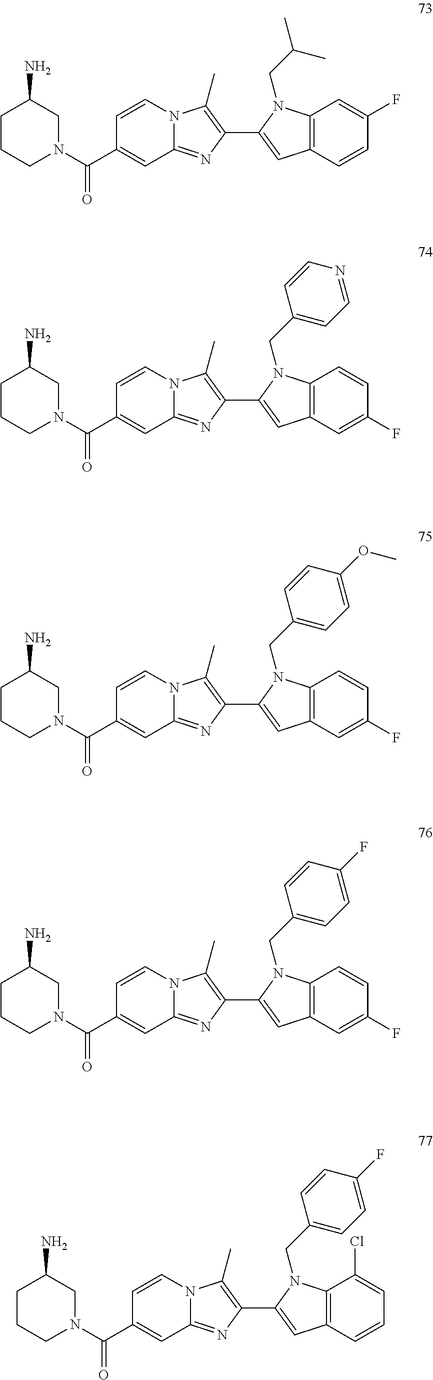 [SMILES: Cc1c(-c2cc3ccc(F)cc3n2CC(C)C)nc2cc(C(=O)N3CCC[C@@H](N)C3)ccn12 ; Cc1c(-c2cc3cc(F)ccc3n2Cc2ccncc2)nc2cc(C(=O)N3CCC[C@@H](N)C3)ccn12 ; COc1ccc(Cn2c(-c3nc4cc(C(=O)N5CCC[C@@H](N)C5)ccn4c3C)cc3cc(F)ccc32)cc1 ; Cc1c(-c2cc3cc(F)ccc3n2Cc2ccc(F)cc2)nc2cc(C(=O)N3CCC[C@@H](N)C3)ccn12 ; Cc1c(-c2cc3cccc(Cl)c3n2Cc2ccc(F)cc2)nc2cc(C(=O)N3CCC[C@@H](N)C3)ccn12]

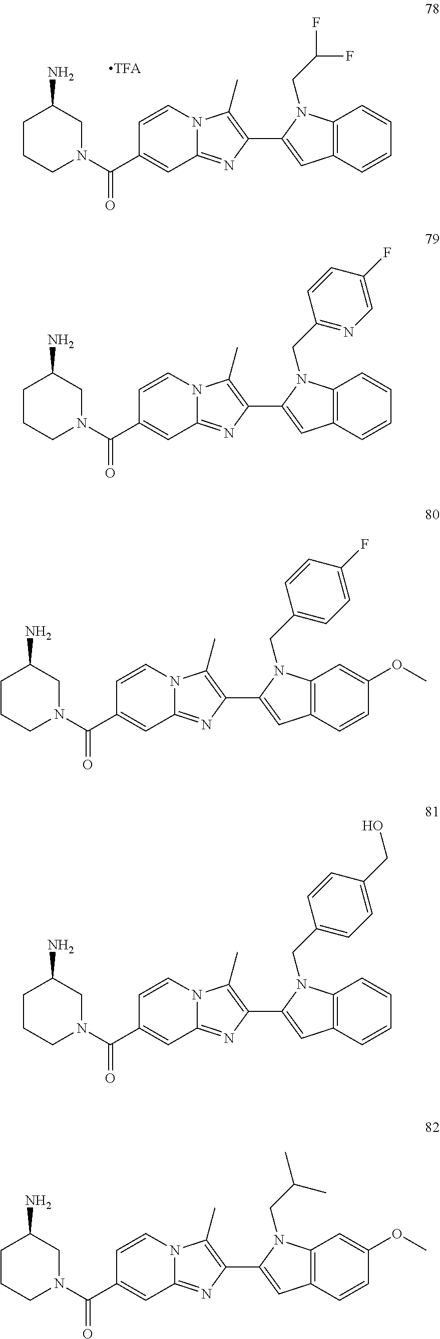 [SMILES: Cc1c(-c2cc3ccccc3n2CC(F)F)nc2cc(C(=O)N3CCC[C@@H](N)C3)ccn12 ; Cc1c(-c2cc3ccccc3n2Cc2ccc(F)cn2)nc2cc(C(=O)N3CCC[C@@H](N)C3)ccn12 ; COc1ccc2cc(-c3nc4cc(C(=O)N5CCC[C@@H](N)C5)ccn4c3C)n(Cc3ccc(F)cc3)c2c1 ; Cc1c(-c2cc3ccccc3n2Cc2ccc(CO)cc2)nc2cc(C(=O)N3CCC[C@@H](N)C3)ccn12 ; COc1ccc2cc(-c3nc4cc(C(=O)N5CCC[C@@H](N)C5)ccn4c3C)n(CC(C)C)c2c1]

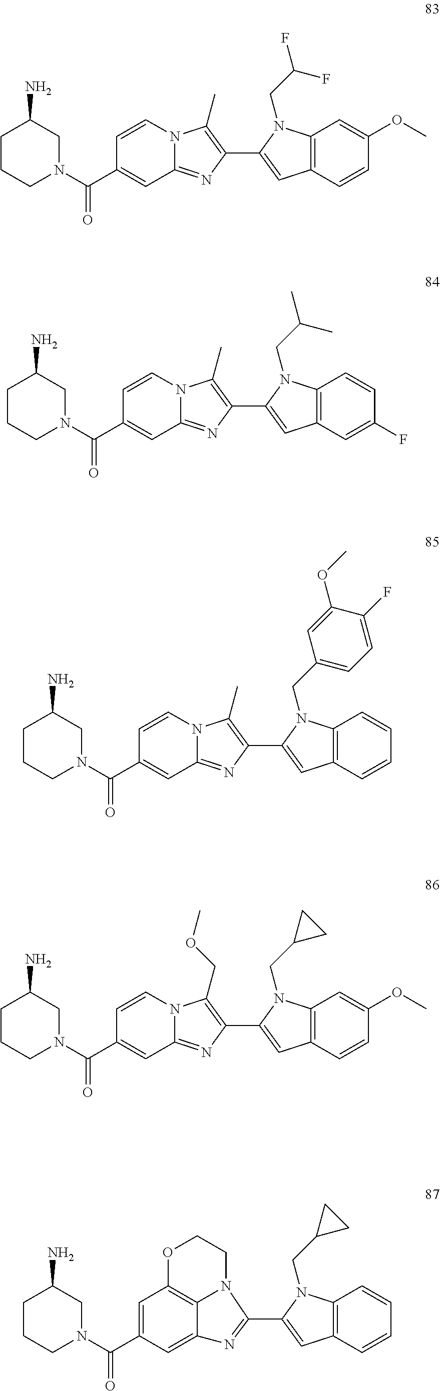 [SMILES: COc1ccc2cc(-c3nc4cc(C(=O)N5CCC[C@@H](N)C5)ccn4c3C)n(CC(F)F)c2c1 ; Cc1c(-c2cc3cc(F)ccc3n2CC(C)C)nc2cc(C(=O)N3CCC[C@@H](N)C3)ccn12 ; COc1cc(Cn2c(-c3nc4cc(C(=O)N5CCC[C@@H](N)C5)ccn4c3C)cc3ccccc32)ccc1F ; COCc1c(-c2cc3ccc(OC)cc3n2CC2CC2)nc2cc(C(=O)N3CCC[C@@H](N)C3)ccn12 ; N[C@@H]1CCCN(C(=O)c2cc3c4c(c2)nc(-c2cc5ccccc5n2CC2CC2)n4CCO3)C1]

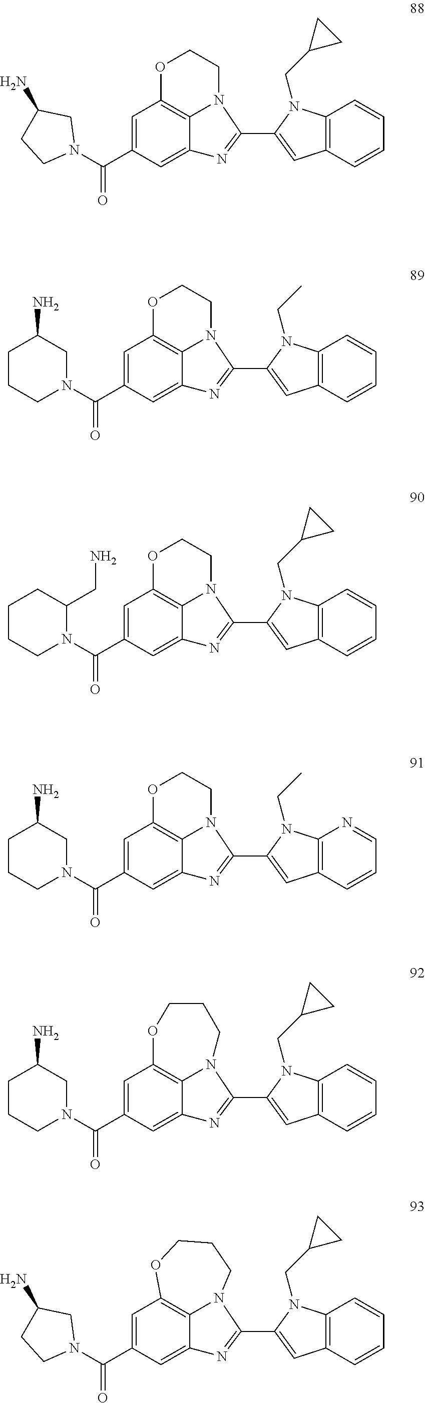 [SMILES: N[C@@H]1CCN(C(=O)c2cc3c4c(c2)nc(-c2cc5ccccc5n2CC2CC2)n4CCO3)C1 ; CCn1c(-c2nc3cc(C(=O)N4CCC[C@@H](N)C4)cc4c3n2CCO4)cc2ccccc21 ; NCC1CCCCN1C(=O)c1cc2c3c(c1)nc(-c1cc4ccccc4n1CC1CC1)n3CCO2 ; CCn1c(-c2nc3cc(C(=O)N4CCC[C@@H](N)C4)cc4c3n2CCO4)cc2cccnc21 ; N[C@@H]1CCCN(C(=O)c2cc3c4c(c2)nc(-c2cc5ccccc5n2CC2CC2)n4CCCO3)C1 ; N[C@@H]1CCN(C(=O)c2cc3c4c(c2)nc(-c2cc5ccccc5n2CC2CC2)n4CCCO3)C1]

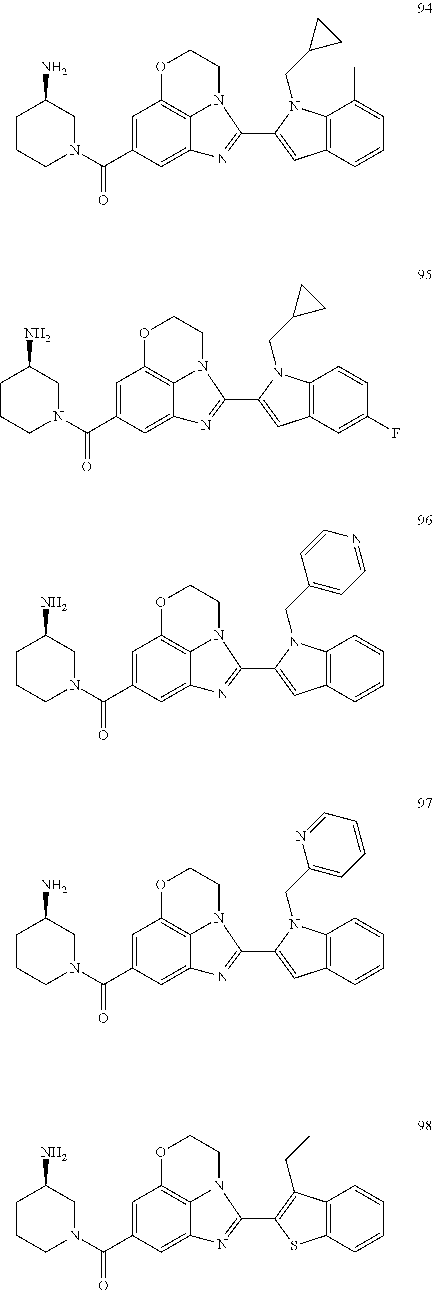 [SMILES: Cc1cccc2cc(-c3nc4cc(C(=O)N5CCC[C@@H](N)C5)cc5c4n3CCO5)n(CC3CC3)c12 ; N[C@@H]1CCCN(C(=O)c2cc3c4c(c2)nc(-c2cc5cc(F)ccc5n2CC2CC2)n4CCO3)C1 ; N[C@@H]1CCCN(C(=O)c2cc3c4c(c2)nc(-c2cc5ccccc5n2Cc2ccncc2)n4CCO3)C1 ; N[C@@H]1CCCN(C(=O)c2cc3c4c(c2)nc(-c2cc5ccccc5n2Cc2ccccn2)n4CCO3)C1 ; CCc1c(-c2nc3cc(C(=O)N4CCC[C@@H](N)C4)cc4c3n2CCO4)sc2ccccc12]

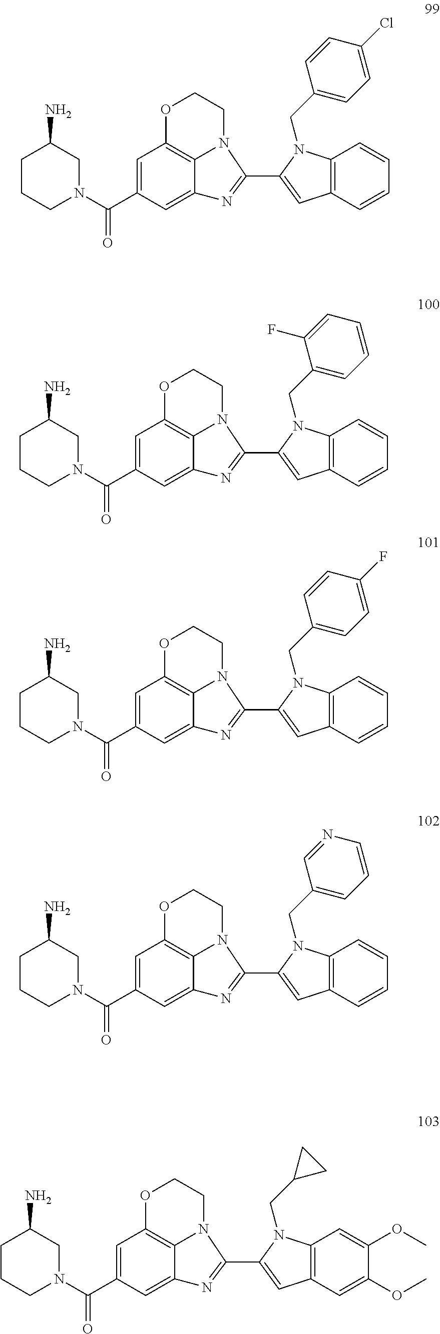 [SMILES: N[C@@H]1CCCN(C(=O)c2cc3c4c(c2)nc(-c2cc5ccccc5n2Cc2ccc(Cl)cc2)n4CCO3)C1 ; N[C@@H]1CCCN(C(=O)c2cc3c4c(c2)nc(-c2cc5ccccc5n2Cc2ccccc2F)n4CCO3)C1 ; N[C@@H]1CCCN(C(=O)c2cc3c4c(c2)nc(-c2cc5ccccc5n2Cc2ccc(F)cc2)n4CCO3)C1 ; N[C@@H]1CCCN(C(=O)c2cc3c4c(c2)nc(-c2cc5ccccc5n2Cc2cccnc2)n4CCO3)C1 ; COc1cc2cc(-c3nc4cc(C(=O)N5CCC[C@@H](N)C5)cc5c4n3CCO5)n(CC3CC3)c2cc1OC]

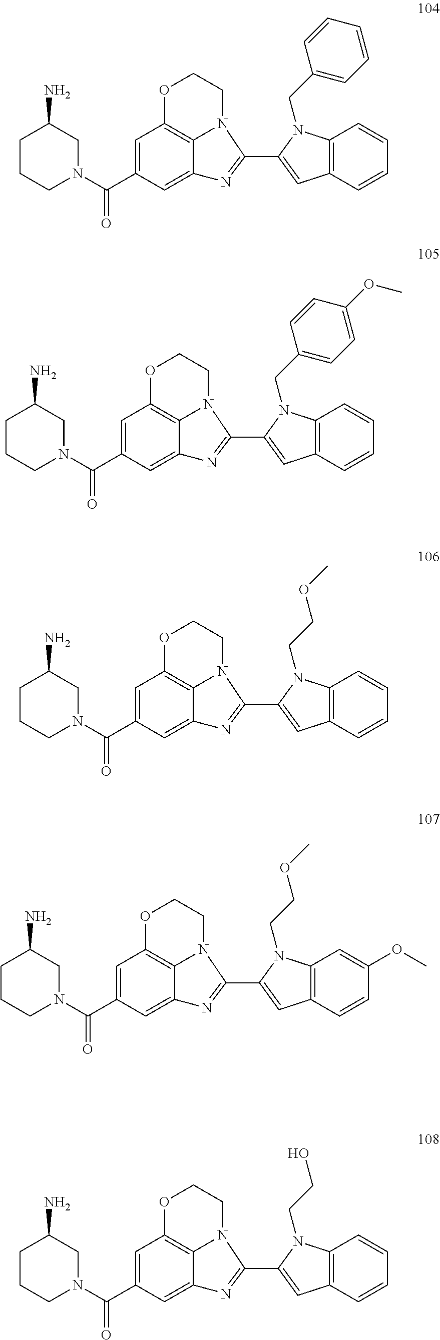 [SMILES: N[C@@H]1CCCN(C(=O)c2cc3c4c(c2)nc(-c2cc5ccccc5n2Cc2ccccc2)n4CCO3)C1 ; COc1ccc(Cn2c(-c3nc4cc(C(=O)N5CCC[C@@H](N)C5)cc5c4n3CCO5)cc3ccccc32)cc1 ; COCCn1c(-c2nc3cc(C(=O)N4CCC[C@@H](N)C4)cc4c3n2CCO4)cc2ccccc21 ; COCCn1c(-c2nc3cc(C(=O)N4CCC[C@@H](N)C4)cc4c3n2CCO4)cc2ccc(OC)cc21 ; N[C@@H]1CCCN(C(=O)c2cc3c4c(c2)nc(-c2cc5ccccc5n2CCO)n4CCO3)C1]

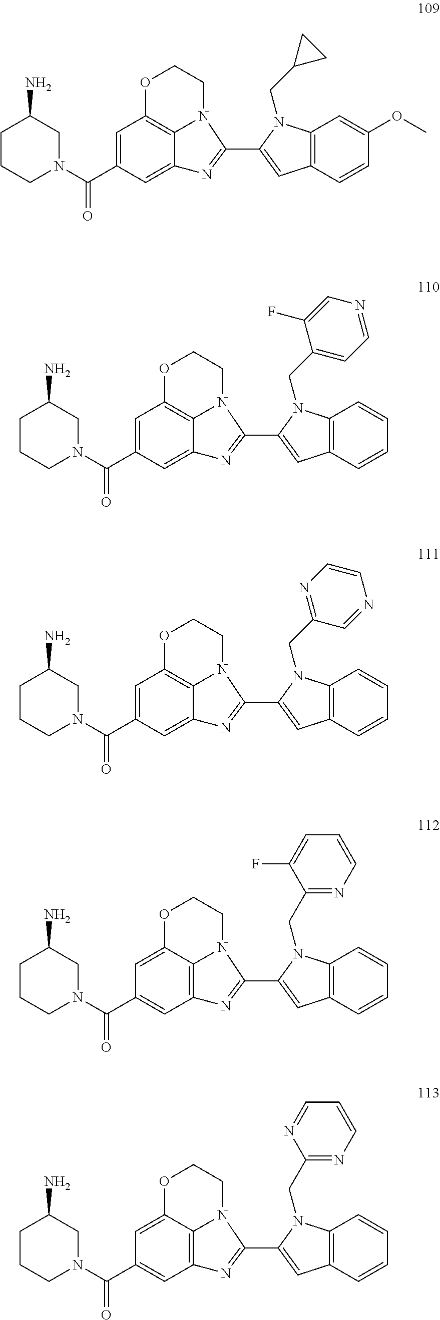 [SMILES: COc1ccc2cc(-c3nc4cc(C(=O)N5CCC[C@@H](N)C5)cc5c4n3CCO5)n(CC3CC3)c2c1 ; N[C@@H]1CCCN(C(=O)c2cc3c4c(c2)nc(-c2cc5ccccc5n2Cc2ccncc2F)n4CCO3)C1 ; N[C@@H]1CCCN(C(=O)c2cc3c4c(c2)nc(-c2cc5ccccc5n2Cc2cnccn2)n4CCO3)C1 ; N[C@@H]1CCCN(C(=O)c2cc3c4c(c2)nc(-c2cc5ccccc5n2Cc2ncccc2F)n4CCO3)C1 ; N[C@@H]1CCCN(C(=O)c2cc3c4c(c2)nc(-c2cc5ccccc5n2Cc2ncccn2)n4CCO3)C1]

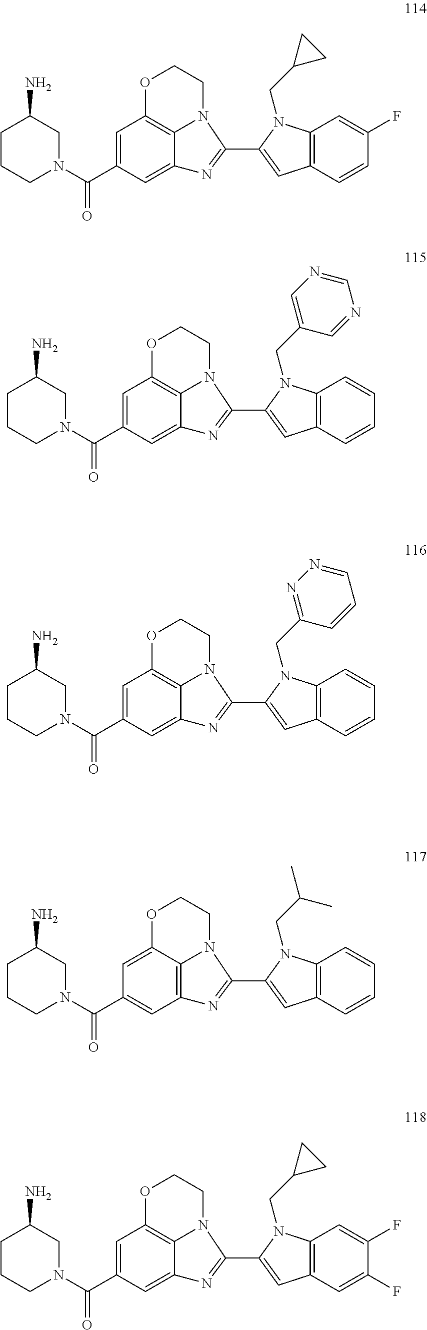 [SMILES: N[C@@H]1CCCN(C(=O)c2cc3c4c(c2)nc(-c2cc5ccc(F)cc5n2CC2CC2)n4CCO3)C1 ; N[C@@H]1CCCN(C(=O)c2cc3c4c(c2)nc(-c2cc5ccccc5n2Cc2cncnc2)n4CCO3)C1 ; N[C@@H]1CCCN(C(=O)c2cc3c4c(c2)nc(-c2cc5ccccc5n2Cc2cccnn2)n4CCO3)C1 ; CC(C)Cn1c(-c2nc3cc(C(=O)N4CCC[C@@H](N)C4)cc4c3n2CCO4)cc2ccccc21 ; N[C@@H]1CCCN(C(=O)c2cc3c4c(c2)nc(-c2cc5cc(F)c(F)cc5n2CC2CC2)n4CCO3)C1]

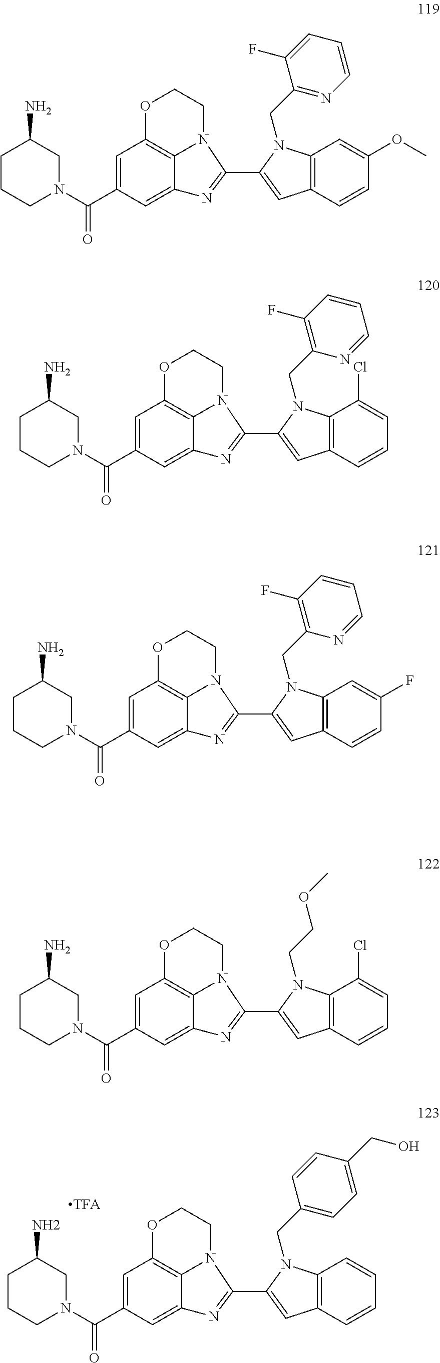 [SMILES: COc1ccc2cc(-c3nc4cc(C(=O)N5CCC[C@@H](N)C5)cc5c4n3CCO5)n(Cc3ncccc3F)c2c1 ; N[C@@H]1CCCN(C(=O)c2cc3c4c(c2)nc(-c2cc5cccc(Cl)c5n2Cc2ncccc2F)n4CCO3)C1 ; N[C@@H]1CCCN(C(=O)c2cc3c4c(c2)nc(-c2cc5ccc(F)cc5n2Cc2ncccc2F)n4CCO3)C1 ; COCCn1c(-c2nc3cc(C(=O)N4CCC[C@@H](N)C4)cc4c3n2CCO4)cc2cccc(Cl)c21 ; N[C@@H]1CCCN(C(=O)c2cc3c4c(c2)nc(-c2cc5ccccc5n2Cc2ccc(CO)cc2)n4CCO3)C1]

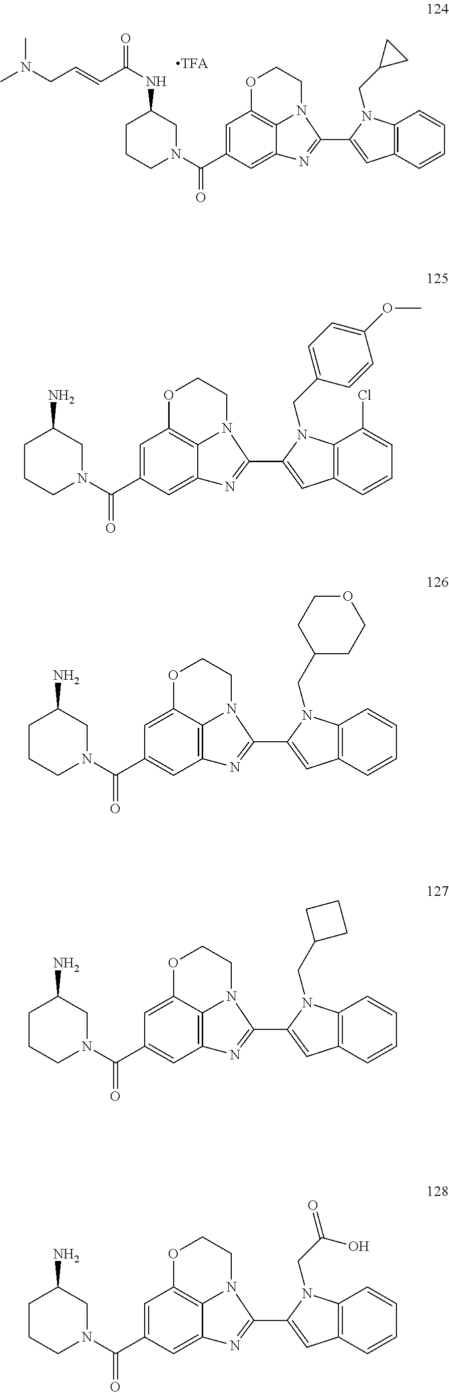 [SMILES: CN(C)C/C=C/C(=O)N[C@@H]1CCCN(C(=O)c2cc3c4c(c2)nc(-c2cc5ccccc5n2CC2CC2)n4CCO3)C1 ; COc1ccc(Cn2c(-c3nc4cc(C(=O)N5CCC[C@@H](N)C5)cc5c4n3CCO5)cc3cccc(Cl)c32)cc1 ; N[C@@H]1CCCN(C(=O)c2cc3c4c(c2)nc(-c2cc5ccccc5n2CC2CCOCC2)n4CCO3)C1 ; N[C@@H]1CCCN(C(=O)c2cc3c4c(c2)nc(-c2cc5ccccc5n2CC2CCC2)n4CCO3)C1 ; N[C@@H]1CCCN(C(=O)c2cc3c4c(c2)nc(-c2cc5ccccc5n2CC(=O)O)n4CCO3)C1]

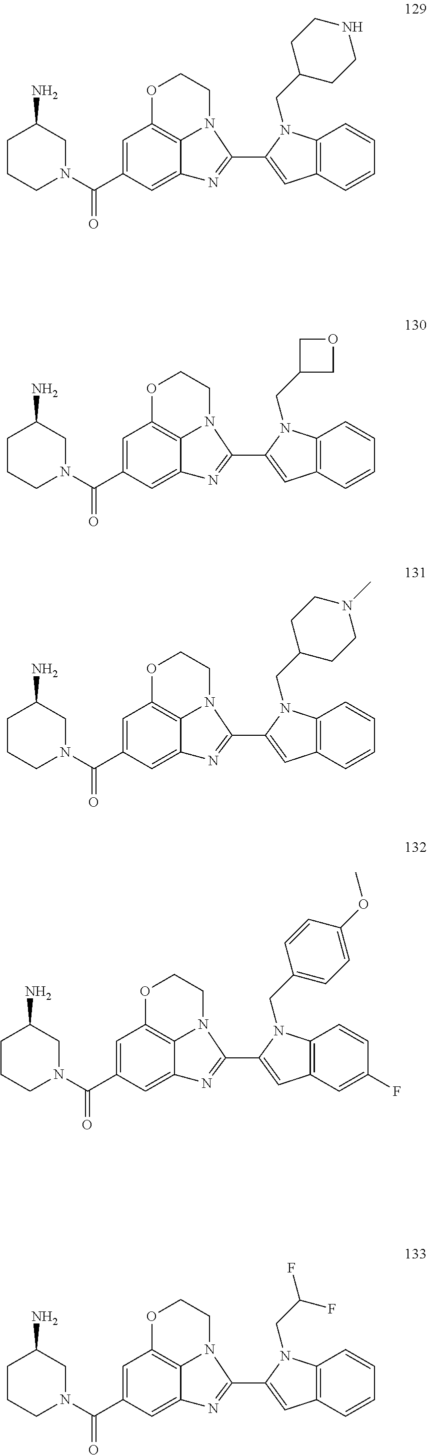 [SMILES: N[C@@H]1CCCN(C(=O)c2cc3c4c(c2)nc(-c2cc5ccccc5n2CC2CCNCC2)n4CCO3)C1 ; N[C@@H]1CCCN(C(=O)c2cc3c4c(c2)nc(-c2cc5ccccc5n2CC2COC2)n4CCO3)C1 ; CN1CCC(Cn2c(-c3nc4cc(C(=O)N5CCC[C@@H](N)C5)cc5c4n3CCO5)cc3ccccc32)CC1 ; COc1ccc(Cn2c(-c3nc4cc(C(=O)N5CCC[C@@H](N)C5)cc5c4n3CCO5)cc3cc(F)ccc32)cc1 ; N[C@@H]1CCCN(C(=O)c2cc3c4c(c2)nc(-c2cc5ccccc5n2CC(F)F)n4CCO3)C1]

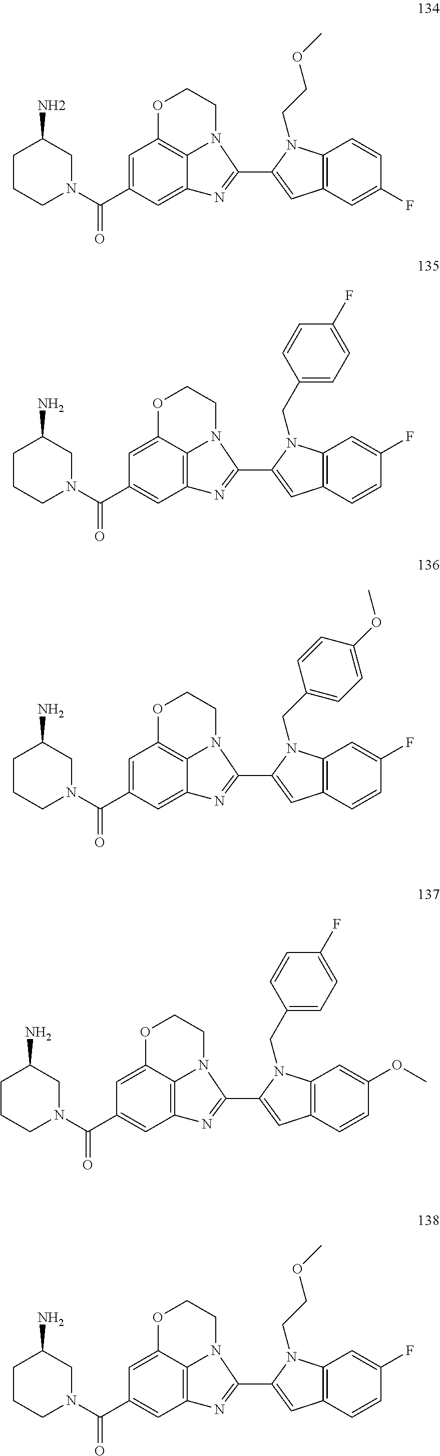 [SMILES: COCCn1c(-c2nc3cc(C(=O)N4CCC[C@@H](N)C4)cc4c3n2CCO4)cc2cc(F)ccc21 ; N[C@@H]1CCCN(C(=O)c2cc3c4c(c2)nc(-c2cc5ccc(F)cc5n2Cc2ccc(F)cc2)n4CCO3)C1 ; COc1ccc(Cn2c(-c3nc4cc(C(=O)N5CCC[C@@H](N)C5)cc5c4n3CCO5)cc3ccc(F)cc32)cc1 ; COc1ccc2cc(-c3nc4cc(C(=O)N5CCC[C@@H](N)C5)cc5c4n3CCO5)n(Cc3ccc(F)cc3)c2c1 ; COCCn1c(-c2nc3cc(C(=O)N4CCC[C@@H](N)C4)cc4c3n2CCO4)cc2ccc(F)cc21]

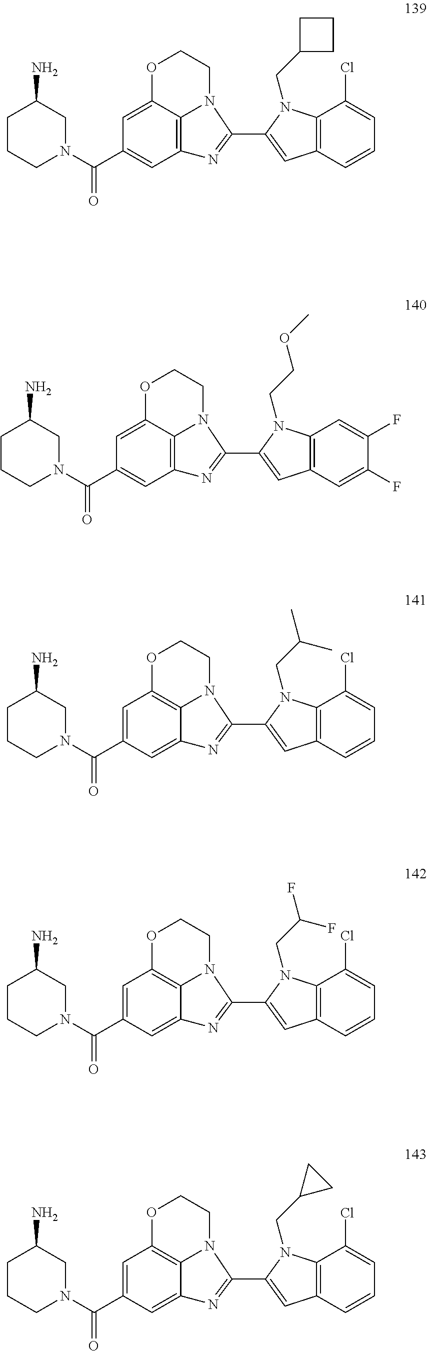 [SMILES: N[C@@H]1CCCN(C(=O)c2cc3c4c(c2)nc(-c2cc5cccc(Cl)c5n2CC2CCC2)n4CCO3)C1 ; COCCn1c(-c2nc3cc(C(=O)N4CCC[C@@H](N)C4)cc4c3n2CCO4)cc2cc(F)c(F)cc21 ; CC(C)Cn1c(-c2nc3cc(C(=O)N4CCC[C@@H](N)C4)cc4c3n2CCO4)cc2cccc(Cl)c21 ; N[C@@H]1CCCN(C(=O)c2cc3c4c(c2)nc(-c2cc5cccc(Cl)c5n2CC(F)F)n4CCO3)C1 ; N[C@@H]1CCCN(C(=O)c2cc3c4c(c2)nc(-c2cc5cccc(Cl)c5n2CC2CC2)n4CCO3)C1]

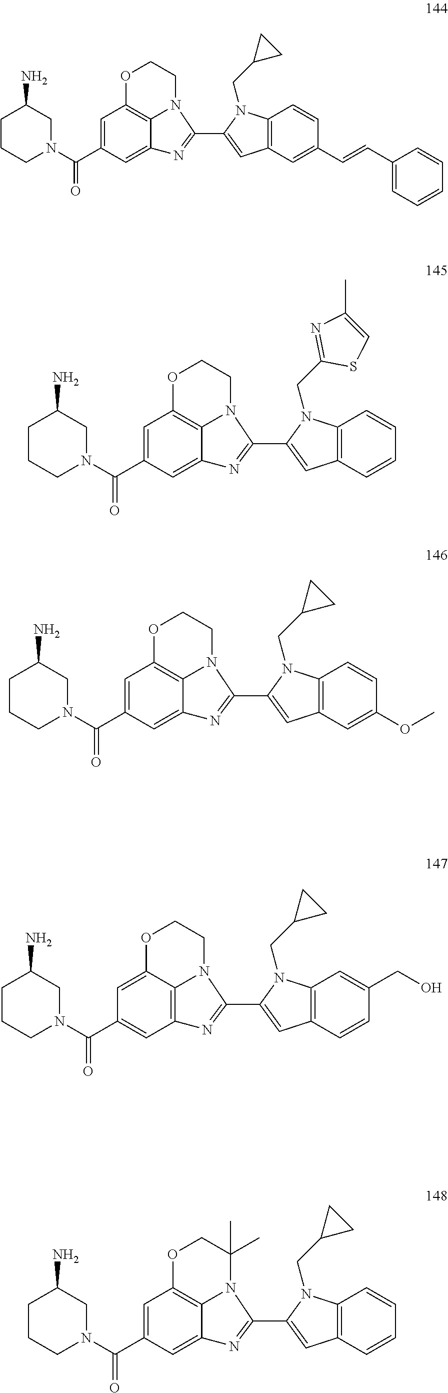 [SMILES: N[C@@H]1CCCN(C(=O)c2cc3c4c(c2)nc(-c2cc5cc(/C=C/c6ccccc6)ccc5n2CC2CC2)n4CCO3)C1 ; Cc1csc(Cn2c(-c3nc4cc(C(=O)N5CCC[C@@H](N)C5)cc5c4n3CCO5)cc3ccccc32)n1 ; COc1ccc2c(c1)cc(-c1nc3cc(C(=O)N4CCC[C@@H](N)C4)cc4c3n1CCO4)n2CC1CC1 ; N[C@@H]1CCCN(C(=O)c2cc3c4c(c2)nc(-c2cc5ccc(CO)cc5n2CC2CC2)n4CCO3)C1 ; CC1(C)COc2cc(C(=O)N3CCC[C@@H](N)C3)cc3nc(-c4cc5ccccc5n4CC4CC4)n1c23]

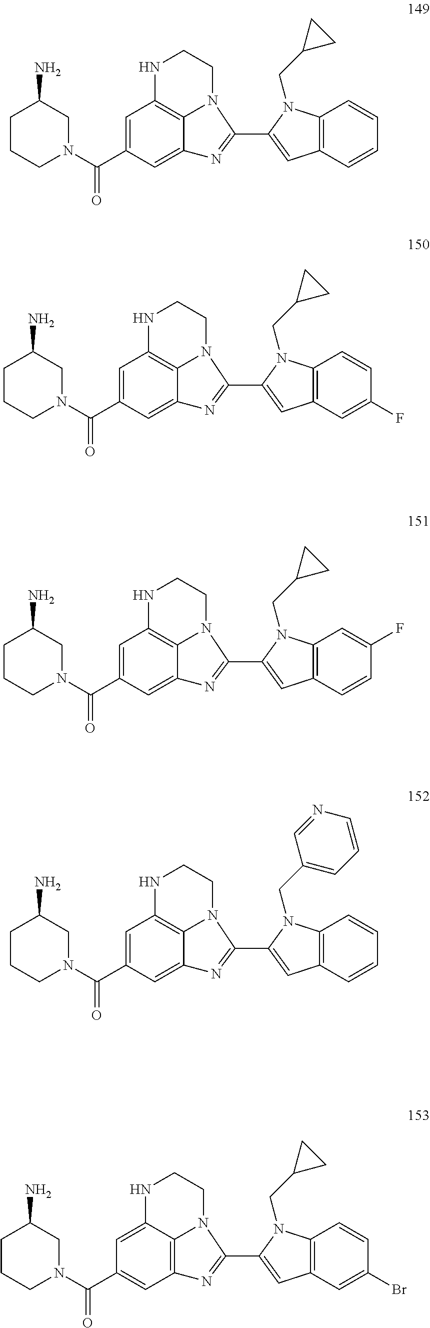 [SMILES: N[C@@H]1CCCN(C(=O)c2cc3c4c(c2)nc(-c2cc5ccccc5n2CC2CC2)n4CCN3)C1 ; N[C@@H]1CCCN(C(=O)c2cc3c4c(c2)nc(-c2cc5cc(F)ccc5n2CC2CC2)n4CCN3)C1 ; N[C@@H]1CCCN(C(=O)c2cc3c4c(c2)nc(-c2cc5ccc(F)cc5n2CC2CC2)n4CCN3)C1 ; N[C@@H]1CCCN(C(=O)c2cc3c4c(c2)nc(-c2cc5ccccc5n2Cc2cccnc2)n4CCN3)C1 ; N[C@@H]1CCCN(C(=O)c2cc3c4c(c2)nc(-c2cc5cc(Br)ccc5n2CC2CC2)n4CCN3)C1]

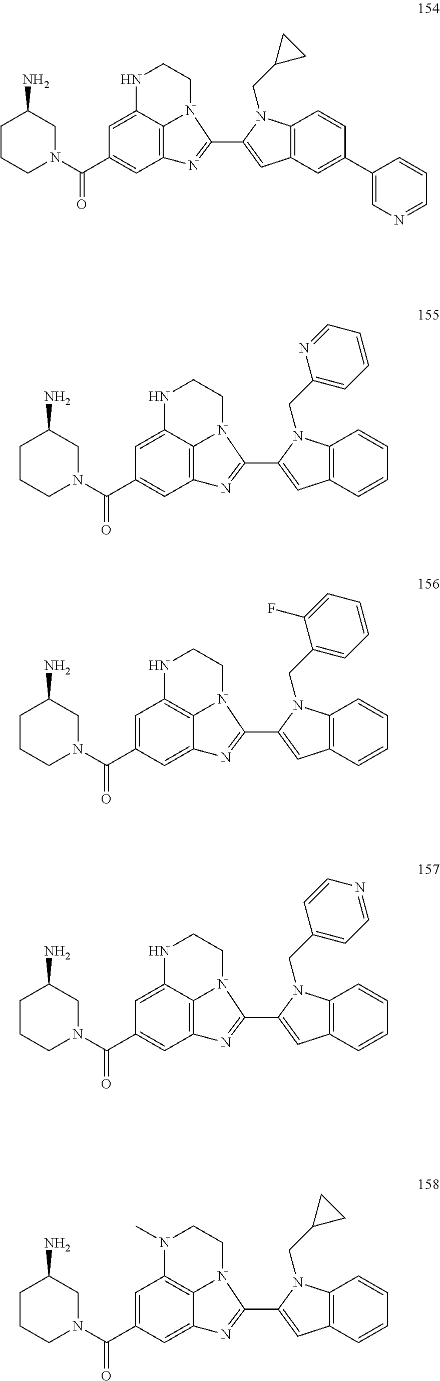 [SMILES: N[C@@H]1CCCN(C(=O)c2cc3c4c(c2)nc(-c2cc5cc(-c6cccnc6)ccc5n2CC2CC2)n4CCN3)C1 ; N[C@@H]1CCCN(C(=O)c2cc3c4c(c2)nc(-c2cc5ccccc5n2Cc2ccccn2)n4CCN3)C1 ; N[C@@H]1CCCN(C(=O)c2cc3c4c(c2)nc(-c2cc5ccccc5n2Cc2ccccc2F)n4CCN3)C1 ; N[C@@H]1CCCN(C(=O)c2cc3c4c(c2)nc(-c2cc5ccccc5n2Cc2ccncc2)n4CCN3)C1 ; CN1CCn2c(-c3cc4ccccc4n3CC3CC3)nc3cc(C(=O)N4CCC[C@@H](N)C4)cc1c32]

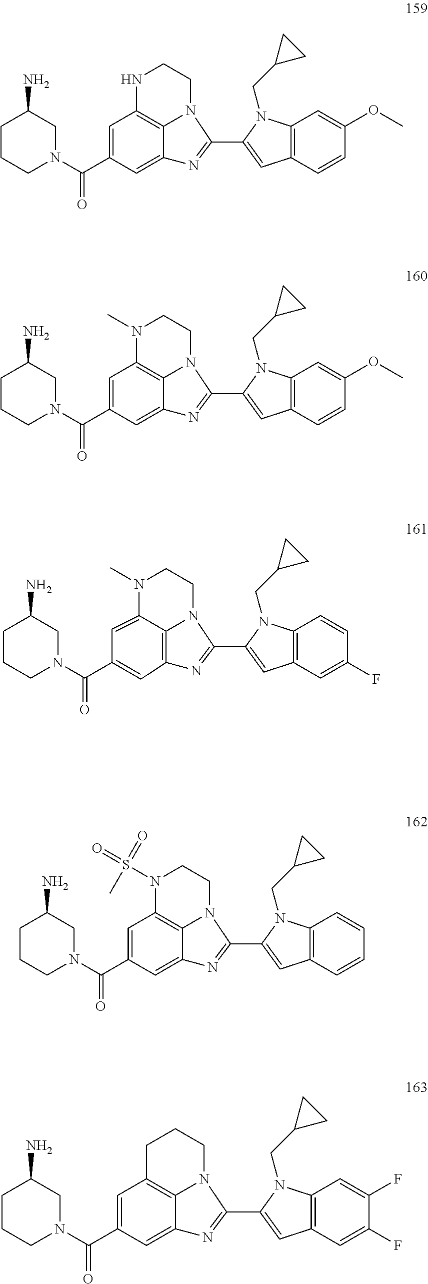 [SMILES: COc1ccc2cc(-c3nc4cc(C(=O)N5CCC[C@@H](N)C5)cc5c4n3CCN5)n(CC3CC3)c2c1 ; COc1ccc2cc(-c3nc4cc(C(=O)N5CCC[C@@H](N)C5)cc5c4n3CCN5C)n(CC3CC3)c2c1 ; CN1CCn2c(-c3cc4cc(F)ccc4n3CC3CC3)nc3cc(C(=O)N4CCC[C@@H](N)C4)cc1c32 ; CS(=O)(=O)N1CCn2c(-c3cc4ccccc4n3CC3CC3)nc3cc(C(=O)N4CCC[C@@H](N)C4)cc1c32 ; N[C@@H]1CCCN(C(=O)c2cc3c4c(c2)nc(-c2cc5cc(F)c(F)cc5n2CC2CC2)n4CCC3)C1]

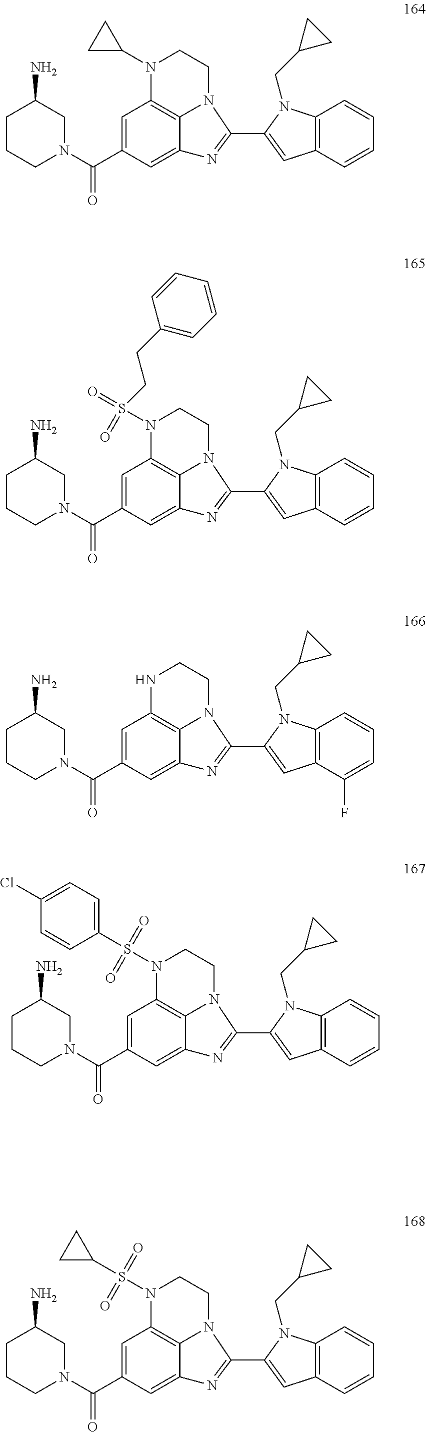 [SMILES: N[C@@H]1CCCN(C(=O)c2cc3c4c(c2)nc(-c2cc5ccccc5n2CC2CC2)n4CCN3C2CC2)C1 ; N[C@@H]1CCCN(C(=O)c2cc3c4c(c2)nc(-c2cc5ccccc5n2CC2CC2)n4CCN3S(=O)(=O)CCc2ccccc2)C1 ; N[C@@H]1CCCN(C(=O)c2cc3c4c(c2)nc(-c2cc5c(F)cccc5n2CC2CC2)n4CCN3)C1 ; N[C@@H]1CCCN(C(=O)c2cc3c4c(c2)nc(-c2cc5ccccc5n2CC2CC2)n4CCN3S(=O)(=O)c2ccc(Cl)cc2)C1 ; N[C@@H]1CCCN(C(=O)c2cc3c4c(c2)nc(-c2cc5ccccc5n2CC2CC2)n4CCN3S(=O)(=O)C2CC2)C1]

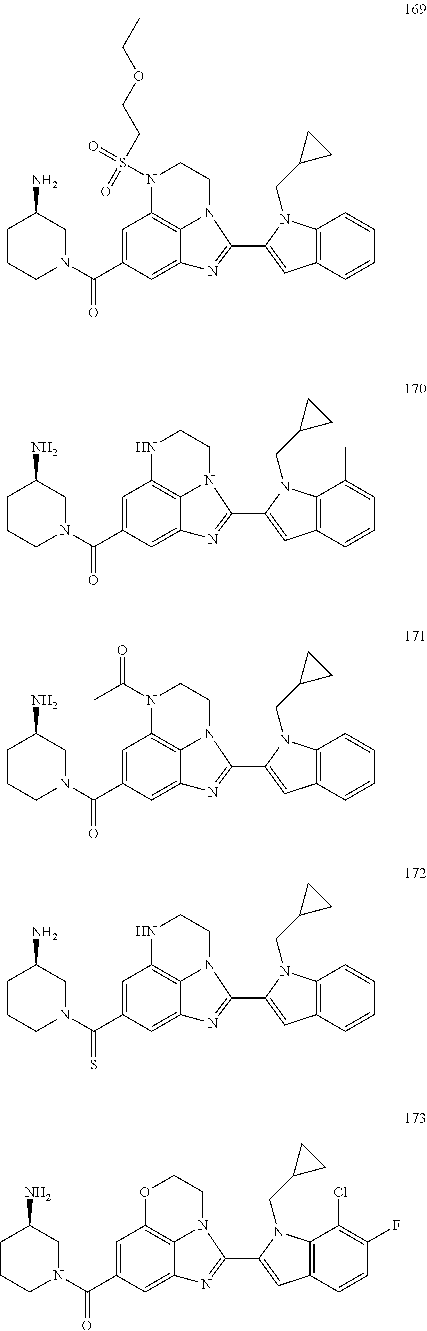 [SMILES: CCOCCS(=O)(=O)N1CCn2c(-c3cc4ccccc4n3CC3CC3)nc3cc(C(=O)N4CCC[C@@H](N)C4)cc1c32 ; Cc1cccc2cc(-c3nc4cc(C(=O)N5CCC[C@@H](N)C5)cc5c4n3CCN5)n(CC3CC3)c12 ; CC(=O)N1CCn2c(-c3cc4ccccc4n3CC3CC3)nc3cc(C(=O)N4CCC[C@@H](N)C4)cc1c32 ; N[C@@H]1CCCN(C(=S)c2cc3c4c(c2)nc(-c2cc5ccccc5n2CC2CC2)n4CCN3)C1 ; N[C@@H]1CCCN(C(=O)c2cc3c4c(c2)nc(-c2cc5ccc(F)c(Cl)c5n2CC2CC2)n4CCO3)C1]

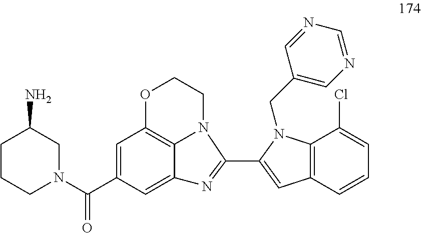 [SMILES: N[C@@H]1CCCN(C(=O)c2cc3c4c(c2)nc(-c2cc5cccc(Cl)c5n2Cc2cncnc2)n4CCO3)C1]

Any of these compounds or a combination of these compounds with a therapeutically effective pharmaceutical composition optionally with a suitable carrier may be administered to a subject in need of treatment for a condition or disease or disorder or condition wherein neutrophils contribute to pathogenesis or worsening of disease. The condition or disease or disorder or condition may be selected from a group consisting of: systemic inflammatory response syndrome (SIRS); acute lung injury (ALI); acute respiratory distress syndrome (ARDS); Anti Neutrophil Cytoplasmic Antibody (ANCA) associated vasculitis (AAV); Hidradenitis suppurativa (HS); multi-organ failure or multi-organ dysfunction syndrome (MODS) from, e.g., ARDS, haemorrhagic shock, surgery, burns, sepsis; sepsis-induced coagulopathy; trauma; multiple sclerosis; acute kidney injury (AKI); AKI-associated tubular necrosis and distant organ injury; post-trauma surgery; haemorrhagic shock; infections, or cytokine storms induced by drugs or any agent; ischemic or haemorrhagic stroke; secondary brain injury in stroke; myocardial ischemia/infarction; atherosclerotic vulnerable plaques; atherosclerotic thrombosis; coronary artery disease; acute coronary syndrome; heart failure; reperfusion injury; comorbidities (e.g., thrombosis and endothelial dysfunction) in kidney dialysis patients; ischemic or drug-induced haemorrhagic transformation in the brain, haemorrhagic encephalopathy, traumatic brain injury; anoxic brain injury, chronic kidney disease; cancer metastasis, breast cancer, prostate cancer, pancreatic cancer, gastric cancer, lung cancer, colon cancer, rectal cancer, esophagus cancer, duodenal cancer, tongue cancer, pharyngeal cancer, brain tumor, neurinoma, clear cell carcinoma, non-small cell lung cancer, small cell lung cancer, liver cancer, kidney cancer, bile duct cancer, uterine body cancer, cervical cancer, ovarian cancer, urinary bladder, skin cancer, hemangioma, malignant lymphoma, malignant melanoma, thyroid cancer, bone tumor, vascular fibroma, glioblastoma, sarcoma, neuroendocrine tumors, retinoblastoma, penile cancer, pediatric solid cancer, renal cell carcinoma, lymphoma, myeloma and leukemia (including, for example acute myelogenous leukemia (AML), chronic myelogenous leukemia (CML), chronic neutrophilic leukemia, chronic eosinophilic leukemia, chronic lymphocytic leukemia (CLL), acute lymphoblastic leukemia (ALL), hairy cell leukemia, cutaneous T-cell lymphoma (CTCL), multiple myeloma (MM), Myeloproliferative neoplasms (MPN), a disease category that includes polycythemia vera (PV), essential thrombocythemia, essential thrombocytosis (ET) and myelofibrosis (MF), chronic myelogenous leukemia (CML), chronic neutrophilic leukemia (CNL), chronic eosinophilic leukemia (CEL), diabetes; type 1 diabetes; type 2 diabetes; angiopathies; vasculopathies; end-organ complications (e.g., retinopathy or diabetic kidney disease); poor wound healing of diabetic ulcers; deep vein thrombosis; cancer; cancer metastasis; systemic microthrombosis; chemotherapy-induced microthrombosis; atherosclerotic thrombosis; systemic lupus erythematosus (SLE); lupus nephritis; SLE-accelerated atherosclerosis; rheumatoid arthritis; COPD; cystic fibrosis; pulmonary disease; Alzheimer's disease; sickle cell disease; inflammatory bowel disease (IBD); Crohn's disease; ulcerative colitis; and indeterminate colitis.

### General Procedure for Biological Evaluation

### Example 1

Citrullination and NETosis

Protein citrullination is an important posttranslational modification in both human disease and gene regulation. Histone H3 Citrullination is the posttranslational deimination of arginine residues to citrullines, catalyzed by the peptidylarginine deiminases (PADs). In neutrophils, the enzyme peptide arginine deiminase 4 (PAD4) is central in the citrullination of histones prior to the externalization of DNA during NETosis. NETosis is a unique form of cell death that is characterized by the release of decondensed chromatin and granular contents to the extracellular space. Neutrophils, the most numerous leukocytes that arrive quickly at the site of an infection, were the first cell type shown to undergo extracellular trap formation. Bacterial breakdown products and inflammatory stimuli induce NETosis and the release of NETs requires enzyme activities. Histones in NET chromatin become modified by peptidylarginine deiminase 4 (PAD4) and cleaved at specific sites by proteases.

Neutrophil Isolation:

Neutrophils were isolated according to the EasySep Direct Human Neutrophil Isolation Kit. In short, 50 mL of blood sample was drawn in a vacutainer containing K₂EDTA from a healthy donor. To the whole blood, the isolation cocktail of 50 μL/mL was added. Then, in the next step 50 μL/mL of RapidSpheres™ was added to the sample. The sample was mixed evenly with the pipette and incubated for 10 minutes at room temperature. After incubation, PBS containing 1 mM EDTA was added at the ratio of 1:1 to the blood. Then the sample tube was incubated for 10 minutes. The enriched cell suspension was transferred into a new tube and RapidSpheres™ were added at 50 μL/mL to this suspension, mixed and incubated for 10 minutes at room temperature. At this step, the enriched cell suspension was carefully collected as a clear fraction into a fresh tube. Then, previous tube was replaced with the fresh tube containing enriched cell suspension. After the final separation, the clear solution of enriched cell suspension was carefully transferred to fresh tube which was ready to use for the experiment. These cells were then subjected to wash twice with plain RPMI 1640 media without Fetal Bovine Serum (FBS) and centrifuged at 1500 rpm for 5 minutes at room temperature. Cell counts were performed on each sample by Invitrogen cell counter. After the cell count, cell density was adjusted according to the experiment and re-suspended in complete RPMI media containing RPMI1640, 2% BSA and 1 mM CaCl₂.

Citrullination ELISA:

Freshly isolated neutrophils from human donor were seeded at a density of 0.5×10⁶ cells in 400 μL cell suspension/well in a 24-well plate and cells were pre-incubated with the compound for 30 minutes by adding 50 μL of the 10× concentration to achieve the final 1× concentration of compound (10 μM, 1 μM and 0.1 μM). 1% DMSO was added in control wells. Then, the cells were stimulated in the presence and absence of calcium ionophore by adding 50 μL/well to achieve the final concentration of 25 μM. Upon incubation for 30 minutes with calcium ionophore, S7 nuclease was added to the wells and incubated for 10 minutes to disrupt NET DNA. Following incubation, 10 μL of 1 mM EDTA was added to inactivate nuclease. Cells were scrapped gently and transferred to the tubes, followed by centrifugation at 1500 rpm for 5 minutes. After centrifugation, supernatant was discarded, and 200 μL of lysis buffer containing PBS and 1× Protease inhibitor was added to the tubes. The obtained lysis was then subjected to mechanical shearing using Geno grinder by spin the plate at 2500 rpm for 10 minutes, followed by collection of supernatant and stored at −20° C. or immediately used for ELISA.

ELISA was performed as per Citrullinated Histone H3 ELISA Kit. All the buffer components from the kit were dissolved according to the manufactures protocol. ELISA Buffer Concentrate (10×) (Item No. 400060) and Wash Buffer Concentrate (400×) (Item No. 400060) along with polysorbate 20 (2000×) (Item No. 400035) was diluted to 1× with milliQ water. The standard for use in the ELISA was prepared as follows:
Obtain eight clean test tubes and label them, #1 through #8. Aliquot 4.9 mL of ELISA Buffer into tube #1.
Aliquot 500 μL of ELISA Buffer into the tubes #2-8. Then, transfer 0.1 mL of freshly prepared stock standard (500 ng/mL) to tube #1.
Mix gently and serially dilute the standard by removing 500 μL from tube #1 and adding into tube #2; Repeat this process for tubes #3-7.
Do not add any CitH3 ELISA Standard to tube #8. This tube is considered as blank, the lowest point on the standard curve.

100 μL of standards or diluted sample was added to the appropriate wells on the 96-well plate. Plates were covered with 96-well cover sheet and incubated for two hours at room temperature on an orbital shaker. The samples and standard were removed from the wells and washed four times with Wash Buffer. After the last wash, Anti-Histone H3 HRP Conjugate (Item No. 501441) (10×) stock solution was diluted in 1× ELISA buffer and 100 μL of the HRP Conjugate working solution was added to each well of the plate and incubated for one hour at room temperature on an orbital shaker. Repeated the wash steps and after the last wash, 100 μL of TMB substrate solution was added to each well of the plate and incubated for 30 minutes at room temperature in the dark on an orbital shaker. After incubation, 100 μL of HRP stop solution was added to each well of the plate. The plates were allowed to develop yellow colour from blue and then they were read at a wavelength of 450 nm in a microplate reader. The IC₅₀ values were subsequently determined using a sigmoidal dose-response curve (variable slope) in GraphPad Prism® 5 software.

Table 1, below, shows the activity of selected compounds of this invention in the Citrullination ELISA assay described above. Compounds having an activity designated as “A” provided IC₅₀<0.1 μM; compounds having an activity designated as “B” provided IC₅₀ 0.1-1 μM; and compounds having an activity designated as “C” provided IC₅₀>1 μM.

TABLE 1

| | Citrullination in |
|---|---|
| Compound | Neutrophils |
| No. | IC₅₀ (uM) |
| 4 | A |
| 10 | A |
| 11 | B |
| 12 | A |
| 13 | B |
| 15 | A |
| 25 | B |
| 26 | A |
| 30 | B |
| 37 | A |
| 39 | A |
| 41 | B |
| 42 | A |
| 44 | A |
| 45 | A |
| 49 | A |
| 51 | C |
| 52 | B |
| 53 | B |
| 54 | A |
| 56 | C |
| 58 | C |
| 60 | C |
| 61 | C |
| 62 | B |
| 67 | C |
| 68 | A |
| 69 | B |
| 70 | A |
| 71 | C |
| 72 | B |
| 74 | C |
| 75 | B |
| 76 | C |
| 78 | C |
| 79 | C |
| 80 | C |
| 81 | C |
| 87 | B |
| 95 | A |
| 97 | B |
| 99 | B |
| 100 | A |
| 102 | A |
| 103 | A |
| 104 | B |
| 105 | B |
| 106 | B |
| 107 | B |
| 109 | B |
| 110 | A |
| 111 | B |
| 112 | A |
| 113 | A |
| 114 | A |
| 117 | B |
| 119 | B |
| 120 | B |
| 121 | C |
| 122 | B |
| 123 | B |
| 125 | A |
| 126 | C |
| 127 | B |
| 132 | C |
| 133 | C |
| 134 | B |
| 135 | A |
| 136 | C |
| 137 | C |
| 141 | C |
| 143 | B |
| 144 | A |
| 145 | A |
| 146 | B |
| 147 | B |
| 149 | B |
| 150 | B |
| 151 | A |
| 152 | A |
| 153 | A |
| 156 | A |
| 159 | B |
| 160 | A |
| 161 | A |
| 163 | A |
| 166 | B |
| 170 | B |
| 171 | A |
| 172 | A |

### Example 2

NETosis Assay:

Freshly isolated neutrophils from human donor were seeded at a density of 0.5×10⁶ cells in 400 μL cell suspension/well in a 24-well plate and cells were pre-incubated with the compound for 30 minutes by adding 50 μL of the 10× concentration to achieve the final 1× concentration of compound (10 μM, 1 μM and 0.1 μM). 1% DMSO was added in control wells. Then, the cells were stimulated in the presence and absence of calcium ionophore by adding 50 μL/well to achieve the final concentration of 25 μM. Upon incubation for 30 minutes with calcium ionophore, S7 nuclease was added to the wells and incubated for 10 minutes to disrupt NET DNA. Following incubation, 10 μL of 1 mM EDTA was added to inactivate nuclease. Cells were scrapped gently and transferred to FACS tubes followed by centrifugation at 1500 rpm for 5 minutes. The pellets obtained were re-suspended in PBS containing 1% BSA and stained with SYTOX green at a final concentration of 50 μM for 10 minutes. Then, cells were acquired in BD FACS Calibur (Total 10000 events/sample) flow cytometer.

Result

Average percent inhibition for compound 143 was calculated as 2% at 0.1 μM, 30% at 1 μM and 85% at 10 μM in two independent NETosis assays using human neutrophils.

### Example 3

Bleomycin Induced Lung Fibrosis Model

Bleomycin (BLM)-induced mice fibrosis is a well-established animal model used to study pathogenesis of lung fibrosis and to estimate the potential anti-fibrotic agents. C57BL/6 male mice at 6-8 weeks of age were purchased from vivo biotech India (under the license from Taconic). The procedures involving the care and use of animals in the study was reviewed and approved by the Institutional Animal Care and Use Committee (IAEC/JDC/2018-161R) prior to conduct. During the study, the care and use of animals was in accordance with the principles outlined in the Guide for the Care and Use of Laboratory Animals, 8th Edition, 2010 (National Research Council) and every effort was made to minimize suffering. The animals were acclimatized for 7 days prior to experimental use. Mice were caged with free access to food and fresh water in a temperature-controlled room (22° C.-24° C.) on a 12 h light/dark cycle. Pulmonary fibrosis was induced by invasive method in animals using single intra-tracheal administration of 50 μg bleomycin prepared in 50 μL of saline under isoflurane anaesthesia. Animals with intra-tracheal administration of 50 μL saline was served as control group. The day of BLM administration was defined as day 0. Mice were administered with test compounds orally from day 0 to 14 for prophylactic intervention. Animals were monitored and evaluated for their body weight loss. Animals were euthanized on Day 14 and lungs were weighed. Fibronectin was measured as a PD biomarker by RT-qPCR and fold change in mRNA level was calculated after normalizing to GAPDH. Lung pathology was performed with Gomori trichrome stain. The Modified Ashcroft Index score was determined for the vehicle-treated control mice and the test compound treated mice.

Result

Lung weight of mice treated with bleomycin showed a significant increase and treatment with compound 143 at 50 mg/kg, PO, BID showed a reduction in lung weight comparable to standard of care Nintedanib dosed at 100 mg/kg, PO, QD. There was a substantial increase in fibronectin mRNA in disease mice as compared to normal mice and compound 143 showed a stronger reduction of fibronectin mRNA as compared to Nintedanib.

### Example 4

Silica Induced Pro-Fibrotic Lung Model

The instillation of silica into the murine lung results in the development of fibrotic nodules that resemble simple silicotic nodular fibrosis, which develop in humans following some occupational exposures to dust and particulate aerosols. Silica can be delivered to rodents via aerosolization, intratracheal administration or oropharyngeal aspiration. C57BL/6 female mice at an age of 6-8 weeks were instilled with silica particles (2.5 mg/mouse) by non-surgical intratracheal (IT) method. The day of silica instillation was defined as day 0. Test compound was administered twice daily and standard of care was dosed once daily from day 7 to 11 for therapeutic intervention. Animals were monitored and evaluated for their clinical signs and body weight loss. Animals were euthanized on day 11 and the lungs were weighed and biomarkers were analyzed in lung. Silica administration to mice by IT route resulted in significant increase in lung weight and associated fibronectin level. Dosing with compound 143 at 50 mg/kg PO, BID resulted in a significant decrease in both lung weight and fibronectin level that was comparable to Nintedanib dosed at 100 mg/kg, PO, QD.

### Example 5

Dextran Sulfate Sodium (DSS) Induced Colitis Model

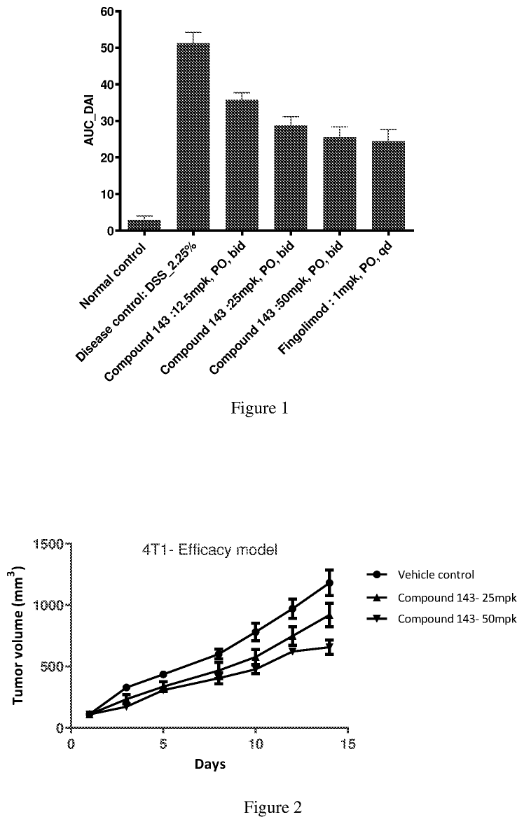

Ulcerative colitis (UC) is an intermittent inflammatory bowel disease of the colonic mucosa, with periods of exacerbated symptoms (the active phase of the disease), and periods that are relatively symptom-free (remission phase of disease). Among various chemical induced colitis models, DSS-induced colitis model is widely used because of its simplicity and many similarities with human ulcerative colitis. C₅₇BL/6 male mice at 6-8 weeks of age were purchased from vivo biotech India (under the license from Taconic). The procedures involving the care and use of animals in the study was reviewed and approved by the Institutional Animal Care and Use Committee (IAEC/JDC/2018-161R) prior to conduct. During the study, the care and use of animals was in accordance with the principles outlined in the Guide for the Care and Use of Laboratory Animals, 8^{th} Edition, 2010 (National Research Council) and every effort was made to minimize suffering. The animals were acclimatized for ˜10 days prior to experimental use. Mice were caged with free access to food and fresh water in a temperature-controlled room (22° C.-24° C.) on a 12 h light/dark cycle. On the day of DSS administration (day 0), labelled (using ear punch or any other convenient method) groups of control and experimental mice should be weighed and, if required, average group weight equilibrated so as to eliminate any significant weight difference between groups. Normal control animals were provided with regular drinking water and disease control animals were provided with 2.25% DSS in drinking water for 5 days (Day 1 to Day 5) and next 5 days, both the groups received regular drinking water (Day 6 to Day 10). DSS was replaced with fresh DSS formulation once in 2.5 days. Animals were dosed with Vehicle (0.5% Methyl cellulose in PBS+0.025% Tween-80), Compound 143, PO, bid and Fingolimod, PO, qd. Administration of 2.25% DSS in drinking water induced significant ulcerative colitis leading to ˜5-fold disease induction in mice of disease control group compared to normal control. Compound 143 at all doses 12.5, 25 & 50 mg/kg, PO BID resulted in 8.7%, 26.6% and 45.5% disease reversal respectively, when compared to disease control (Figure. 1). Oral dosing of Fingolimod (1 mg/kg, QD) attenuated the DSS-induced elevated DAI, with disease reversal of ˜58.9% when compared to disease control on day 10 (FIG. 1).

### Example 6

Murine Breast Cancer Efficacy Model

PAD4 is highly expressed in malignant tumours of various cell types, Here, the mouse mammary carcinoma of 4T1 and EMT6 cells were used as an experimental model. Mouse 4T1, and EMT-6, a breast cancer cell line was sourced from American Type Culture Collection (ATCC), USA. Cells were grown according to the product information. To establish allografts, the cells were harvested by trypsinization when they reached around 70 to 80% confluence. After harvesting, Balb/C female mice (7-8 weeks) were implanted with 4T1 (1 million cells/site) and EMT-6 (0.5 million cells/site) subcutaneously in the dorsal right flank of mice using a 1 mL BD syringe attached to a 24-gauge needle. When the tumors became palpable tumor measurement was initiated and at an approximate tumor volume of ˜110 and 93 mm3, respectively. Animals were randomized into different treatment groups keeping tumor volume and the number of animals/group similar across the groups and compound 143 treatment was initiated. The procedures involving the care and use of animals in the study was reviewed and approved by the Institutional Animal Care and Use Committee (IAEC/JDC/2021/246R) prior to conduct. During the study, the care and use of animals was in accordance with the principles outlined in the Guide for the Care and Use of Laboratory Animals, 8^{th} Edition, 2010 (National Research Council) and every effort was made to minimize suffering. The animals were acclimatized for 7 days prior to experimental use. Mice were caged with free access to food and fresh water in a temperature-controlled room (22° C.-24° C.) on a 12 h light/dark cycle.

Tumor dimensions (length and breadth) were measured on days 1 (animal randomization based on tumor volume) and thrice weekly thereafter until study termination.

Tumor volumes were calculated using the formula (b2*1)*0.52 where 1=length, b=breadth (Dusan Djokovic et al., BMC Cancer, 2010, 10:641).

The percent tumor growth inhibition (% TGI) as a function of anti-tumor efficacy were computed with respect to untreated vehicle control by comparing to the tumor volumes of day 1. Compound 143 group was administered by oral gavage twice daily for 14 days for 4T1 bearing mice and 13 days for EMT-6 bearing mice. For 4T1 (FIG. 1) on day 14, TGI with compound 143 (25 mg/kg, BID, PO) and (50 mg/kg, BID, PO) dosing was 24.3 and 48.8%, as compared to their respective vehicle controls. Similarly, for EMT-6 on day 13, dosing with compound 143 (50 mg/kg, BID, PO) result in a TGI of 42.6%, as compared to their respective vehicle controls. There were no significant body weight changes observed in both 4T1 and EMT-6 groups (FIG. 2).

1. A method for treating and/or preventing a disease or disorder or condition associated with NETosis, the method comprising: administering to a subject a compound selected from a group consisting of Formula (I), (R)-(3-aminopiperidin-1-yl)(2-(3-ethylbenzo[b]thiophen-2-yl)-3-methylimidazo[1,2-a]pyridin-7-yl)methanone (19), (R)-(3-aminopiperidin-1-yl)(2-(1-ethyl-5-phenyl-1H-pyrrol-2-yl)-3-methylimidazo[1,2-a]pyridin-7-yl)methanone (22), (R)-(3-aminopiperidin-1-yl)(2-(2-ethylphenyl)-3-methylimidazo[1,2-a]pyridin-7-yl)methanone (31), and (R)-(3-aminopiperidin-1-yl)(2-(3-ethylbenzo[b]thiophen-2-yl)-3,4-dihydro-5-oxa-1,2a-diazaacenaphthylen-7-yl)methanone (98), or its polymorph, stereoisomer, prodrug, solvate, co-crystal, intermediate, pharmaceutically acceptable salt, metabolite or composition thereof; 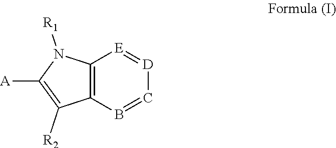
wherein,
A represents 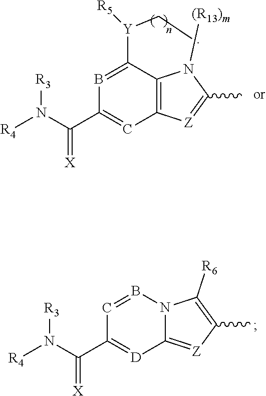
n is 0-2;
X is selected from 0 or S;
Y is selected from O, N, S, S(O), SO₂ or C;
Z is selected from N or CR₇;
B is selected from N or CR₈;
C is selected from N or CR₉;
D is selected from N or CR₁₀;
E is selected from N or CR₁₁;
R₁ is selected from a group consisting of hydrogen, C₁₋₆ alkyl, C₁₋₆ haloalkyl, C₁₋₆ alkoxy, C₃₋₆ cycloalkyl, C(O)C₁₋₆ alkyl, C(O)C₁₋₆ haloalkyl, C(O)NR₁₂, C(O)C₁₋₆ alkylamino, SO₂C₁₋₆ alkyl, SO₂C₁₋₆ haloalkyl, SO₂NR₁₂, SO₂NC₁₋₆ alkylamino, C₆ al, C₁₋₆ heteroaryl, C(O)C₆ aryl, C(O)C₁₋₆ heteroaryl, SO₂C₆ aryl, and SO₂C₁₋₆ heteroaryl;
wherein C₁₋₆ alkyl, C₁₋₆ alkoxy, C₃₋₆ cycloalkyl, C₆ aryl, C₁₋₆ heteroaryl, (CO)C₁₋₆ alkyl, C(O)C₁₋₆ haloalkyl, or SO₂C₁₋₆ alkyl, is optionally substituted with one or more groups selected from hydrogen, C₁₋₆ alkyl, C₁₋₆ alkoxy, C₃₋₈ cycloalkyl, C₆ aryl, C₁₋₆ heteroaryl, C₁₋₆ heterocyclyl, halogen, hydroxyl, —COOH, and cyano;
wherein C₁₋₆ alkyl, C₁₋₆ alkoxy, C₃₋₈ cycloalkyl, C₆ aryl, C₁₋₆ heteroaryl, C₁₋₆ heterocyclyl optionally further substituted with one or more substituents selected from halogen, cyano, oxo, C₁₋₆ alkyl, C₁₋₆ alkoxy, and C₁₋₆ alkylhydroxy;
R₂, R₆, R₈, R₉, R₁₀, R₁₁, and R₁₃ are independently selected from hydrogen, hydroxyl, cyano, amino, halogen, C₁₋₆ alkyl, C₂₋₆ alkenyl, C₁₋₆ alkoxy, C₃₋₆ cycloalkyl, C₁₋₆ haloalkyl, C₁₋₆ haloalkoxy, C₁₋₆ acylamino, C₁₋₆ alkylamino, C₆₋₁₀ aryl, C₁₋₆ heterocyclyl, or C₁₋₆ heteroaryl;
wherein, C₁₋₆ alkyl, C₂₋₆ alkenyl, C₁_s alkoxy, C₁₋₆ heteroaryl, and C₁₋₆ haloalkoxy, is optionally substituted with one or more of the groups selected from hydrogen, oxo, C₁₋₆ alkyl, C₁₋₆ alkoxy, C₁₋₆ haloalkyl, C₃₋₆ cycloalkyl, C₆₋₁₀ aryl, C₁₋₆ heterocyclyl, C₁₋₆ heteroaryl, cyano, and hydroxyl;
m is 0 to 2;
R₃ is hydrogen;
R₄ is selected from C₁₋₆ alkylamino, and 5-10 membered monocyclic or bicyclic saturated heterocyclic ring with 1-3 heteroatoms selected from N, S or 0;
or R₃ and R₄ can be taken together to form a 5-10 membered monocyclic or bicyclic saturated or unsaturated heterocyclic ring with 1-5 heteroatoms selected from N, S or 0;
wherein, the 5-10 membered monocyclic or bicyclic saturated or unsaturated heterocyclic ring is optionally substituted with one or more substituents selected from a group consisting of amino, C₁₋₆ alkylamino, C₁₋₆ acylamino, —NHC(NH)CH₂Cl, —NHC(O)CH═CH—CH₂—N(CH₃)₂, C₁-6 alkyl, halogen, C₁₋₆ alkoxy, and hydroxyl;
R₅ is absent or is selected from a group consisting of hydrogen, C₁₋₆ alkyl, C₃₋₆ cycloalkyl, C₁₋₆ alkylamino, C₁₋₆ haloalkyl, C(O)C₁₋₆ alkyl, C(O)C₁₋₆ haloalkyl, C(O)NR₁₂, C(O)C₁₋₆ alkylamino, SO₂C₁₋₆ alkyl, SO₂C₁₋₆ haloalkyl, SO₂C₃₋₆ cycloalkyl, SO₂NR₁₂, SO₂NC₁₋₆ alkylamino, C₆ aryl, C₁₋₆ heteroaryl, C(O)C₆ aryl, C(O)C₁₋₆ heteroaryl, SO₂C₆ aryl, and SO₂C₁₋₆ heteroaryl;
wherein, C₁₋₆ alkyl, (CO)C₁₋₆ alkyl, C(O)C₁₋₆ haloalkyl, SO₂C₆ aryl, and SO₂C₁₋₆ alkyl, is optionally substituted with C₁₋₆ alkoxy, halogen, C₆ aryl, or C₁₋₆ heteroaryl;
R₇ is selected from a group consisting of hydrogen, hydroxyl, cyano, halogen, C₁₋₆ alkyl, C₁₋₆ alkoxy, C₁₋₆ haloalkyl, C₁₋₆ haloalkoxy, C₁₋₆ acylamino, C₁₋₆ alkylamino, C₆ aryl, and C₁₋₆ heteroaryl;
wherein, C₁₋₆ alkoxy, and C₁₋₆ haloalkoxy, is optionally substituted with one or more of the groups selected from hydrogen, C₁₋₆ alkyl, C₁₋₆ alkoxy, C₁₋₆ haloalkyl, C₁₋₆ alkylhydroxy, cyano, and hydroxyl; and
R₁₂ is independently selected from hydrogen, and C₁₋₆ alkyl.

2. A method for treatment of a disease or disorder or condition ameliorated by inhibition of NETosis, the method comprising: administering to a subject a compound selected from a group consisting of Formula (I), (R)-(3-aminopiperidin-1-yl)(2-(3-ethylbenzo[b]thiophen-2-yl)-3-methylimidazo[1,2-a]pyridin-7-yl)methanone (19), (R)-(3-aminopiperidin-1-yl)(2-(1-ethyl-5-phenyl-1H-pyrrol-2-yl)-3-methylimidazo[1,2-a]pyridin-7-yl)methanone (22), (R)-(3-aminopiperidin-1-yl)(2-(2-ethylphenyl)-3-methylimidazo[1,2-a]pyridin-7-yl)methanone (31), and (R)-(3-aminopiperidin-1-yl)(2-(3-ethylbenzo[b]thiophen-2-yl)-3,4-dihydro-5-oxa-1,2a-diazaacenaphthylen-7-yl)methanone (98), or its polymorph, stereoisomer, prodrug, solvate, co-crystal, intermediate, pharmaceutically acceptable salt, metabolite or composition thereof; 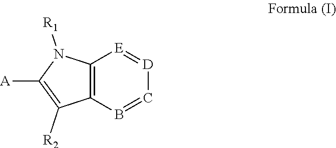
wherein,
A represents 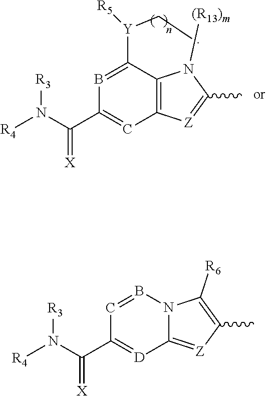
n is 0-2;
X is selected from O or S;
Y is selected from O, N, S, S(O), SO₂ or C;
Z is selected from N or CR₇;
B is selected from N or CR₈;
C is selected from N or CR₉;
D is selected from N or CR₁₀;
E is selected from N or CR₁₁;
R₁ is selected from a group consisting of hydrogen, C₁₋₆ alkyl, C₁₋₆ haloalkyl, C₁₋₆ alkoxy, C₃₋₆ cycloalkyl, C(O)C₁₋₆ alkyl, C(O)C₁₋₆ haloalkyl, C(O)NR₁₂, C(O)C₁₋₆ alkylamino, SO₂C₁₋₆ alkyl, SO₂C₁₋₆ haloalkyl, SO₂NR₁₂, SO₂NC₁₋₆ alkylamino, C₆ al, C₁₋₆ heteroaryl, C(O)C₆ aryl, C(O)C₁₋₆ heteroaryl, SO₂C₆ aryl, and SO₂C₁₋₆ heteroaryl;
wherein, C₁₋₆ alkyl, C₁₋₆ alkoxy, C₃₋₆ cycloalkyl, C₆ aryl, C₁₋₆ heteroaryl, (CO)C₁₋₆ alkyl, C(O)C₁₋₆ haloalkyl, and SO₂C₁₋₆ alkyl, is optionally substituted with one or more groups selected from hydrogen, C₁₋₆ alkyl, C₁₋₆ alkoxy, C₃₋₈ cycloalkyl, C₆ aryl, C₁₋₆ heteroaryl, C₁₋₆ heterocyclyl, halogen, hydroxyl, —COOH, and cyano, and wherein C₁₋₆ alkyl, C₁₋₆ alkoxy, C₃₋₈ cycloalkyl, C₆ aryl, C₁₋₆ heteroaryl, C₁₋₆ heterocyclyl optionally further substituted one or more substituents selected from halogen, cyano, oxo, C₁₋₆ alkyl, C₁₋₆ alkoxy, and C₁₋₆ alkylhydroxy;
R₂, R₆, R₈, R₉, R₁₀, R₁₁, and R₁₃ are independently selected from hydrogen, hydroxyl, cyano, amino, halogen, C₁₋₆ alkyl, C₂₋₆ alkenyl, C₁₋₆ alkoxy, C₃₋₆ cycloalkyl, C₁₋₆ haloalkyl, C₁₋₆ haloalkoxy, C₁₋₆ acylamino, C₁₋₆ alkylamino, C₆₋₁₀ aryl, C₁₋₆ heterocyclyl, or C₁₋₆ heteroaryl;
wherein, C₁₋₆ alkyl, C₂₋₆ alkenyl, C₁₋₆ alkoxy, C₁₋₆ heteroaryl, or C₁₋₆ haloalkoxy, is optionally substituted with one or more of the groups selected from hydrogen, oxo, C₁₋₆ alkyl, C₁₋₆ alkoxy, C₁₋₆ haloalkyl, C₃₋₆ cycloalkyl, C₆₋₁₀ aryl, C₁₋₆ heterocyclyl, C₁_s heteroaryl, cyano, and hydroxyl;
m is 0 to 2;
R₃ is hydrogen;
R₄ is selected from C₁₋₆ alkylamino, and 5-10 membered monocyclic or bicyclic saturated heterocyclic ring with 1-3 heteroatoms selected from N, S or 0;
or R₃ and R₄ can be taken together to form a 5-10 membered monocyclic or bicyclic saturated or unsaturated heterocyclic ring with 1-5 heteroatoms selected from N, S or 0;
wherein, the 5-10 membered monocyclic or bicyclic saturated or unsaturated heterocyclic ring is optionally substituted with one or more substituents selected from a group consisting of amino, C₁₋₆ alkylamino, C₁₋₆ acylamino, —NHC(NH)CH₂Cl, —NHC(O)CH═CH—CH₂—N(CH₃)₂, C₁-6 alkyl, halogen, C₁₋₆ alkoxy, and hydroxyl;
R₅ is absent or is selected from a group consisting of hydrogen, C₁₋₆ alkyl, C₃₋₆ cycloalkyl, C₁₋₆ alkylamino, C₁₋₆ haloalkyl, C(O)C₁₋₆ alkyl, C(O)C₁₋₆ haloalkyl, C(O)NR₁₂, C(O)C₁₋₆ alkylamino, SO₂C₁₋₆ alkyl, SO₂C₁₋₆ haloalkyl, SO₂C₃₋₆ cycloalkyl, SO₂NR₁₂, SO₂NC₁₋₆ alkylamino, C₆ al, C₁₋₆ heteroaryl, C(O)C₆ aryl, C(O)C₁₋₆ heteroaryl, SO₂C₆ aryl, and SO₂C₁₋₆ heteroaryl;
wherein, C₁₋₆ alkyl, (CO)C₁₋₆ alkyl, C(O)C₁₋₆ haloalkyl, SO₂C₆ aryl, and SO₂C₁₋₆ alkyl, is optionally substituted with C₁₋₆ alkoxy, halogen, C₆ aryl, and C₁₋₆ heteroaryl;
R₇ is selected from a group consisting of hydrogen, hydroxyl, cyano, halogen, C₁₋₆ alkyl, C₁₋₆ alkoxy, C₁₋₆ haloalkyl, C₁₋₆ haloalkoxy, C₁₋₆ acylamino, C₁₋₆ alkylamino, C₆ aryl, and C₁₋₆ heteroaryl;
wherein, C₁₋₆ alkoxy, and C₁₋₆ haloalkoxy, is optionally substituted with one or more of the groups selected from hydrogen, C₁₋₆ alkyl, C₁₋₆ alkoxy, C₁₋₆ haloalkyl, C₁₋₆ alkylhydroxy, cyano, and hydroxyl; and
R₁₂ is independently selected from hydrogen, and C₁₋₆ alkyl.

3. The method as claimed in claim 1, wherein the compound is selected from a group consisting of:
(R)-(3-aminopiperidin-1-yl)(2-(3-bromo-1-(cyclopropylmethyl)-1H-indol-2-yl)-3-methylimidazo[1,2-a]pyridin-7-yl)methanone (1),
(R)-(3-aminopiperidin-1-yl)(2-(1-(cyclopropylmethyl)-1H-indol-2-yl)-3-methylimidazo[1,2-a]pyridin-7-yl)methanone (2),
(R)-(3-aminopiperidin-1-yl)(2-(1-benzyl-1H-indol-2-yl)-3-methylimidazo[1,2-a]pyridin-7-yl)methanone (3),
(R)-(3-aminopiperidin-1-yl)(2-(1-ethyl-1H-indol-2-yl)-3-methylimidazo[1,2-a]pyridin-7-yl)methanone (4),
(R)-(3-aminopiperidin-1-yl)(2-(1-ethyl-3-phenyl-1H-indol-2-yl)-3-methylimidazo[1,2-a]pyridin-7-yl)methanone (5),
(R)-(3-aminopyrrolidin-1-yl)(2-(1-ethyl-3-phenyl-1H-indol-2-yl)-3-methylimidazo[1,2-a]pyridin-7-yl)methanone (6),
(R)-(3-aminopyrrolidin-1-yl)(2-(1-ethyl-1H-indol-2-yl)-3-methylimidazo[1,2-a]pyridin-7-yl)methanone (7),
(R)-(3-aminopyrrolidin-1-yl)(2-(1-(cyclopropylmethyl)-1H-indol-2-yl)-3-methylimidazo[1,2-a]pyridin-7-yl)methanone (8),
(2-(1-(cyclopropylmethyl)-1H-indol-2-yl)-3-methylimidazo[1,2-a]pyridin-7-yl)(hexahydro-2H-pyrido[4,3-b][1,4]oxazin-6(5H)-yl)methanone (9),
(R)-(3-aminopiperidin-1-yl)(3-methyl-2-(1-(pyridin-4-ylmethyl)-1H-indol-2-yl)imidazo[1,2-a]pyridin-7-yl)methanone (10),
(R)-(3-aminopiperidin-1-yl)(3-methyl-2-(1-((tetrahydro-2H-pyran-4-yl)methyl)-1H-indol-2-yl)imidazo[1,2-a]pyridin-7-yl)methanone (11),
(R)-(3-aminopiperidin-1-yl)(2-(1-(4-methoxybenzyl)-1H-indol-2-yl)-3-methylimidazo[1,2-a]pyridin-7-yl)methanone (12),
(R)-(3-aminopiperidin-1-yl)(2-(1-(4-fluorobenzyl)-1H-indol-2-yl)-3-methylimidazo[1,2-a]pyridin-7-yl)methanone (13),
(R)-4-((2-(7-(3-aminopiperidine-1-carbonyl)-3-methylimidazo[1,2-a]pyridin-2-yl)-1H-indol-1-yl)methyl)benzonitrile (14),
(R)-(3-aminopiperidin-1-yl)(3-methyl-2-(1-(pyridin-3-ylmethyl)-1H-indol-2-yl)imidazo[1,2-a]pyridin-7-yl)methanone (15),
(R)-(3-aminopiperidin-1-yl)(3-methyl-2-(1-(pyridin-2-ylmethyl)-1H-indol-2-yl)imidazo[1,2-a]pyridin-7-yl)methanone (16),
(R)-(3-aminopiperidin-1-yl)(3-methyl-2-(1-(2,2,2-trifluoroethyl)-1H-indol-2-yl)imidazo[1,2-a]pyridin-7-yl)methanone (17),
(R)-4-((2-(7-(3-aminopiperidine-1-carbonyl)-3-methylimidazo[1,2-a]pyridin-2-yl)-1H-indol-1-yl)methyl)-1-methylpyridin-2(1H)-one (18),
(R)-(3-aminopiperidin-1-yl)(2-(3-ethylbenzo[b]thiophen-2-yl)-3-methylimidazo[1,2-a]pyridin-7-yl)methanone (19),
(R)-(3-aminopiperidin-1-yl)(2-(1-(4-chlorobenzyl)-1H-indol-2-yl)-3-methylimidazo[1,2-a]pyridin-7-yl)methanone (20),
(R)-(3-aminopiperidin-1-yl)(2-(1-(2-fluorobenzyl)-1H-indol-2-yl)-3-methylimidazo[1,2-a]pyridin-7-yl)methanone (21),
(R)-(3-aminopiperidin-1-yl)(2-(1-ethyl-5-phenyl-1H-pyrrol-2-yl)-3-methylimidazo[1,2-a]pyridin-7-yl)methanone (22),
(R)-(3-aminopiperidin-1-yl)(2-(1-(cyclopropylmethyl)-6-methoxy-1H-indol-2-yl)-3-methylimidazo[1,2-a]pyridin-7-yl)methanone (23),
(R)-(3-aminopiperidin-1-yl)(2-(1-ethyl-1H-pyrrolo[2,3-b]pyridin-2-yl)-3-methylimidazo[1,2-a]pyridin-7-yl)methanone (24),
(R)-(3-aminopiperidin-1-yl)(2-(1-(cyclopropylmethyl)-1H-indol-2-yl)-3,5-dimethylimidazo[1,2-a]pyridin-7-yl)methanone (25),
(R)-(3-aminopiperidin-1-yl)(2-(1-benzyl-1H-indol-2-yl)-3,5-dimethylimidazo[1,2-a]pyridin-7-yl)methanone (26),
(R)-(3-aminopiperidin-1-yl)(3,5-dimethyl-2-(1-(pyridin-2-ylmethyl)-1H-indol-2-yl)imidazo[1,2-a]pyridin-7-yl)methanone (27),
(R)-(3-aminopiperidin-1-yl)(2-(1-(cyclopropylmethyl)-6-fluoro-1H-indol-2-yl)-3-methylimidazo[1,2-a]pyridin-7-yl)methanone (28),
(R)-(3-aminopiperidin-1-yl)(2-(1-(cyclopropylmethyl)-5-fluoro-1H-indol-2-yl)-3-methylimidazo[1,2-a]pyridin-7-yl)methanone (29),
(R)-(3-aminopiperidin-1-yl)(2-(1-(cyclopropylmethyl)-7-methyl-1H-indol-2-yl)-3-methylimidazo[1,2-a]pyridin-7-yl)methanone (30),
(R)-(3-aminopiperidin-1-yl)(2-(2-ethylphenyl)-3-methylimidazo[1,2-a]pyridin-7-yl)methanone (31),
(R)-(3-aminopiperidin-1-yl)(2-(1-(cyclopropylmethyl)-1H-indol-2-yl)-3-phenylimidazo[1,2-a]pyridin-7-yl)methanone (32),
(R)-(3-aminopiperidin-1-yl)(3-cyclopropyl-2-(1-(cyclopropylmethyl)-1H-indol-2-yl)imidazo[1,2-a]pyridin-7-yl)methanone (33),
(R)-(3-aminopiperidin-1-yl)(2-(1-(cyclopropylmethyl)-1H-indol-2-yl)-3-(methoxymethyl)imidazo[1,2-a]pyridin-7-yl)methanone (34),
(R)-(3-aminopiperidin-1-yl)(2-(1-(3-fluorobenzyl)-1H-indol-2-yl)-3-methylimidazo[1,2-a]pyridin-7-yl)methanone (35),
(R)-(3-aminopiperidin-1-yl)(3-methyl-2-(1-(thiophen-3-ylmethyl)-1H-indol-2-yl)imidazo[1,2-a]pyridin-7-yl)methanone (36),
(R)-(3-aminopiperidin-1-yl)(2-(1-(furan-3-ylmethyl)-1H-indol-2-yl)-3-methylimidazo[1,2-a]pyridin-7-yl)methanone (37),
(R)-3-aminopiperidin-1-yl)(2-(1-(1-(4-fluorophenyl)ethyl)-1H-indol-2-yl)-3-methylimidazo[1,2-a]pyridin-7-yl)methanone (38),
(R)-(3-aminopiperidin-1-yl)(2-(1-ethyl-5-fluoro-1H-indol-2-yl)-3-methylimidazo[1,2-a]pyridin-7-yl)methanone (39),
(R)-(3-aminopiperidin-1-yl)(2-(1-(cyclopropylmethyl)-4-fluoro-1H-indol-2-yl)-3-methylimidazo[1,2-a]pyridin-7-yl)methanone (40),
(R)-(3-aminopiperidin-1-yl)(3-methyl-2-(1-((4-methylthiazol-2-yl)methyl)-1H-indol-2-yl)imidazo[1,2-a]pyridin-7-yl)methanone (41),
(R)-(3-aminopiperidin-1-yl)(2-(1-(3-methoxybenzyl)-1H-indol-2-yl)-3-methylimidazo[1,2-a]pyridin-7-yl)methanone (42),
(R)-(3-aminopiperidin-1-yl)(2-(5-bromo-1-(cyclopropylmethyl)-1H-indol-2-yl)-3-methylimidazo[1,2-a]pyridin-7-yl)methanone (43),
(R)-(3-aminopiperidin-1-yl)(2-(7-chloro-1-(cyclopropylmethyl)-1H-indol-2-yl)-3-methylimidazo[1,2-a]pyridin-7-yl)methanone (44),
(R)-(3-aminopiperidin-1-yl)(2-(1-(4-fluorobenzyl)-1H-indol-2-yl)-3-methylimidazo[1,2-a]pyridin-7-yl)methanethione (45),
(R)-(3-aminopiperidin-1-yl)(3-methyl-2-(1-methyl-1H-indol-2-yl)imidazo[1,2-a]pyridin-7-yl)methanone (46),
(R)-(3-aminopiperidin-1-yl)(2-(1-(cyclopropylmethyl)-7-methoxy-1H-indol-2-yl)-3-methylimidazo[1,2-a]pyridin-7-yl)methanone (47),
(R)-(3-aminopiperidin-1-yl)(2-(1-((2,4-dimethylthiazol-5-yl)methyl)-1H-indol-2-yl)-3-methylimidazo[1,2-a]pyridin-7-yl)methanone (48),
(R)-(3-aminopiperidin-1-yl)(3-methyl-2-(1-((2-methylthiazol-5-yl)methyl)-1H-indol-2-yl)imidazo[1,2-a]pyridin-7-yl)methanone (49),
(R)-(3-aminopiperidin-1-yl) (2-(1-(3-methoxybenzyl)-1H-indol-2-yl)-3-methylimidazo[1,2-a]pyridin-7-yl)methanone trifluoroacetic acid salt (50),
(R)-(3-aminopiperidin-1-yl)(2-(6-methoxy-1-(pyridin-3-ylmethyl)-1H-indol-2-yl)-3-methylimidazo[1,2-a]pyridin-7-yl)methanone (51),
(R)-(3-aminopiperidin-1-yl)(2-(1-(cyclopropylmethyl)-6-fluoro-1H-indol-2-yl)-3-methylimidazo[1,2-a]pyridin-7-yl)methanone trifluoroacetic acid salt (52),
(R)-(3-aminopiperidin-1-yl)(2-(5-fluoro-1-(3-methoxybenzyl)-1H-indol-2-yl)-3-methylimidazo[1,2-a]pyridin-7-yl)methanone (53),
(R)-(3-aminopiperidin-1-yl)(2-(6-fluoro-1-(4-fluorobenzyl)-1H-indol-2-yl)-3-methylimidazo[1,2-a]pyridin-7-yl)methanone (54),
(R)-(3-aminopiperidin-1-yl)(2-(5,6-difluoro-1-(3-methoxybenzyl)-1H-indol-2-yl)-3-methylimidazo[1,2-a]pyridin-7-yl)methanone (55),
(R,E)-4-(dimethylamino)-N-(1-(2-(1-(4-fluorobenzyl)-1H-indol-2-yl)-3-methylimidazo[1,2-a]pyridine-7-carbonyl)piperidin-3-yl)but-2-enamide (56),
(R)-(3-aminopiperidin-1-yl)(3-methyl-2-(1-(pyrazin-2-ylmethyl)-1H-indol-2-yl)imidazo[1,2-a]pyridin-7-yl)methanone (57),
(R)-(3-aminopiperidin-1-yl)(3-methyl-2-(1-(pyrimidin-5-ylmethyl)-1H-indol-2-yl)imidazo[1,2-a]pyridin-7-yl)methanone trifluoroacetic acid salt (58),
(R)-(3-aminopiperidin-1-yl)(3-methyl-2-(1-(pyridazin-3-ylmethyl)-1H-indol-2-yl)imidazo[1,2-a]pyridin-7-yl)methanone (59),
(R)-(3-aminopiperidin-1-yl)(2-(1-isobutyl-1H-indol-2-yl)-3-methylimidazo[1,2-a]pyridin-7-yl)methanone (60),
(R)-(3-aminopiperidin-1-yl)(2-(1-(cyclobutylmethyl)-1H-indol-2-yl)-3-methylimidazo[1,2-a]pyridin-7-yl)methanone (61),
(R)-(3-aminopiperidin-1-yl)(2-(1-((3-fluoropyridin-2-yl)methyl)-1H-indol-2-yl)-3-methylimidazo[1,2-a]pyridin-7-yl)methanone (62),
(R)-(3-aminopiperidin-1-yl)(2-(1-((5-methoxypyridin-2-yl)methyl)-1H-indol-2-yl)-3-methylimidazo[1,2-a]pyridin-7-yl)methanone (63),
(R)-(3-aminopiperidin-1-yl)(2-(1-(2-methoxyethyl)-1H-indol-2-yl)-3-methylimidazo[1,2-a]pyridin-7-yl)methanone (64),
(R)-(3-aminopiperidin-1-yl)(2-(1-(2-hydroxyethyl)-1H-indol-2-yl)-3-methylimidazo[1,2-a]pyridin-7-yl)methanone (65),
(R)-(3-aminopiperidin-1-yl)(2-(6-methoxy-1-(pyridin-4-ylmethyl)-1H-indol-2-yl)-3-methylimidazo[1,2-a]pyridin-7-yl)methanone (66),
(R)-(3-aminopiperidin-1-yl)(2-(1-ethyl-6-methoxy-1H-indol-2-yl)-3-methylimidazo[1,2-a]pyridin-7-yl)methanone (67),
(R)-(3-aminopiperidin-1-yl)(2-(6-fluoro-1-(pyridin-4-ylmethyl)-1H-indol-2-yl)-3-methylimidazo[1,2-a]pyridin-7-yl)methanone (68),
(R)-(3-aminopiperidin-1-yl)(2-(6-fluoro-1-(pyridin-3-ylmethyl)-1H-indol-2-yl)-3-methylimidazo[1,2-a]pyridin-7-yl)methanone (69),
(R)-(3-aminopiperidin-1-yl)(2-(6-fluoro-1-(3-methoxybenzyl)-1H-indol-2-yl)-3-methylimidazo[1,2-a]pyridin-7-yl)methanone (70),
(R)-(3-aminopiperidin-1-yl)(2-(6-fluoro-1-(4-methoxybenzyl)-1H-indol-2-yl)-3-methylimidazo[1,2-a]pyridin-7-yl)methanone (71),
(R)-(3-aminopiperidin-1-yl)(2-(1-ethyl-6-fluoro-1H-indol-2-yl)-3-methylimidazo[1,2-a]pyridin-7-yl)methanone (72),
(R)-(3-aminopiperidin-1-yl)(2-(6-fluoro-1-isobutyl-1H-indol-2-yl)-3-methylimidazo[1,2-a]pyridin-7-yl)methanone (73),
(R)-(3-aminopiperidin-1-yl)(2-(5-fluoro-1-(pyridin-4-ylmethyl)-1H-indol-2-yl)-3-methylimidazo[1,2-a]pyridin-7-yl)methanone (74),
(R)-(3-aminopiperidin-1-yl)(2-(5-fluoro-1-(4-methoxybenzyl)-1H-indol-2-yl)-3-methylimidazo[1,2-a]pyridin-7-yl)methanone (75),
(R)-(3-aminopiperidin-1-yl)(2-(5-fluoro-1-(4-fluorobenzyl)-1H-indol-2-yl)-3-methylimidazo[1,2-a]pyridin-7-yl)methanone (76),
(R)-(3-aminopiperidin-1-yl)(2-(7-chloro-1-(4-fluorobenzyl)-1H-indol-2-yl)-3-methylimidazo[1,2-a]pyridin-7-yl)methanone (77),
(R)-(3-aminopiperidin-1-yl)(2-(1-(2,2-difluoroethyl)-1H-indol-2-yl)-3-methylimidazo[1,2-a]pyridin-7-yl)methanone trifluoroacetic acid salt (78),
(R)-(3-aminopiperidin-1-yl)(2-(1-((5-fluoropyridin-2-yl)methyl)-1H-indol-2-yl)-3-methylimidazo[1,2-a]pyridin-7-yl)methanone (79),
(R)-(3-aminopiperidin-1-yl)(2-(1-(4-fluorobenzyl)-6-methoxy-1H-indol-2-yl)-3-methylimidazo[1,2-a]pyridin-7-yl)methanone (80),
(R)-(3-aminopiperidin-1-yl)(2-(1-(4-(hydroxymethyl)benzyl)-1H-indol-2-yl)-3-methylimidazo[1,2-a]pyridin-7-yl)methanone (81),
(R)-(3-aminopiperidin-1-yl)(2-(1-isobutyl-6-methoxy-1H-indol-2-yl)-3-methylimidazo[1,2-a]pyridin-7-yl)methanone (82),
(R)-(3-aminopiperidin-1-yl)(2-(1-(2,2-difluoroethyl)-6-methoxy-1H-indol-2-yl)-3-methylimidazo[1,2-a]pyridin-7-yl)methanone (83),
(R)-(3-aminopiperidin-1-yl)(2-(5-fluoro-1-isobutyl-1H-indol-2-yl)-3-methylimidazo[1,2-a]pyridin-7-yl)methanone (84),
(R)-(3-aminopiperidin-1-yl)(2-(1-(4-fluoro-3-methoxybenzyl)-1H-indol-2-yl)-3-methylimidazo[1,2-a]pyridin-7-yl)methanone (85),
(R)-(3-aminopiperidin-1-yl)(2-(1-(cyclopropylmethyl)-6-methoxy-1H-indol-2-yl)-3-(methoxymethyl)imidazo[1,2-a]pyridin-7-yl)methanone (86),
(R)-(3-aminopiperidin-1-yl)(2-(1-(cyclopropylmethyl)-1H-indol-2-yl)-3,4-dihydro-5-oxa-1,2a-diazaacenaphthylen-7-yl)methanone (87),
(R)-(3-aminopyrrolidin-1-yl)(2-(1-(cyclopropylmethyl)-1H-indol-2-yl)-3,4-dihydro-5-oxa-1,2a-diazaacenaphthylen-7-yl)methanone (88),
(R)-(3-aminopiperidin-1-yl)(2-(1-ethyl-1H-indol-2-yl)-3,4-dihydro-5-oxa-1,2a-diazaacenaphthylen-7-yl)methanone (89),
(2-(aminomethyl)piperidin-1-yl)(2-(1-(cyclopropylmethyl)-1H-indol-2-yl)-3,4-dihydro-5-oxa-1,2a-diazaacenaphthylen-7-yl)methanone (90),
(R)-(3-aminopiperidin-1-yl)(2-(1-ethyl-1H-pyrrolo[2,3-b]pyridin-2-yl)-3,4-dihydro-5-oxa-1,2a-diazaacenaphthylen-7-yl)methanone (91),
(R)-(3-aminopiperidin-1-yl)(1-(1-(cyclopropylmethyl)-1H-indol-2-yl)-8,9-dihydro-7H-6-oxa-2,9a-diazabenzo[cd]azulen-4-yl)methanone (92),
(R)-(3-aminopyrrolidin-1-yl)(1-(1-(cyclopropylmethyl)-1H-indol-2-yl)-8,9-dihydro-7H-6-oxa-2,9a-diazabenzo[cd]azulen-4-yl)methanone (93),
(R)-(3-aminopiperidin-1-yl)(2-(1-(cyclopropylmethyl)-7-methyl-1H-indol-2-yl)-3,4-dihydro-5-oxa-1,2a-diazaacenaphthylen-7-yl)methanone (94),
(R)-(3-aminopiperidin-1-yl)(2-(1-(cyclopropylmethyl)-5-fluoro-1H-indol-2-yl)-3,4-dihydro-5-oxa-1,2a-diazaacenaphthylen-7-yl)methanone (95),
(R)-(3-aminopiperidin-1-yl)(2-(1-(pyridin-4-ylmethyl)-1H-indol-2-yl)-3,4-dihydro-5-oxa-1,2a-diazaacenaphthylen-7-yl)methanone (96),
(R)-(3-aminopiperidin-1-yl)(2-(1-(pyridin-2-ylmethyl)-1H-indol-2-yl)-3,4-dihydro-5-oxa-1,2a-diazaacenaphthylen-7-yl)methanone (97),
(R)-(3-aminopiperidin-1-yl)(2-(3-ethylbenzo[b]thiophen-2-yl)-3,4-dihydro-5-oxa-1,2a-diazaacenaphthylen-7-yl)methanone (98),
(R)-(3-aminopiperidin-1-yl)(2-(1-(4-chlorobenzyl)-1H-indol-2-yl)-3,4-dihydro-5-oxa-1,2a-diazaacenaphthylen-7-yl)methanone (99),
(R)-(3-aminopiperidin-1-yl)(2-(1-(2-fluorobenzyl)-1H-indol-2-yl)-3,4-dihydro-5-oxa-1,2a-diazaacenaphthylen-7-yl)methanone (100),
(R)-(3-aminopiperidin-1-yl)(2-(1-(4-fluorobenzyl)-1H-indol-2-yl)-3,4-dihydro-5-oxa-1,2a-diazaacenaphthylen-7-yl)methanone (101),
(R)-(3-aminopiperidin-1-yl)(2-(1-(pyridin-3-ylmethyl)-1H-indol-2-yl)-3,4-dihydro-5-oxa-1,2a-diazaacenaphthylen-7-yl)methanone (102),
(R)-(3-aminopiperidin-1-yl)(2-(1-(cyclopropylmethyl)-5,6-dimethoxy-1H-indol-2-yl)-3,4-dihydro-5-oxa-1,2a-diazaacenaphthylen-7-yl)methanone (103),
(R)-(3-aminopiperidin-1-yl)(2-(1-benzyl-1H-indol-2-yl)-3,4-dihydro-5-oxa-1,2a-diazaacenaphthylen-7-yl)methanone (104),
(R)-(3-aminopiperidin-1-yl)(2-(1-(4-methoxybenzyl)-1H-indol-2-yl)-3,4-dihydro-5-oxa-1,2a-diazaacenaphthylen-7-yl)methanone (105),
(R)-(3-aminopiperidin-1-yl)(2-(1-(2-methoxyethyl)-1H-indol-2-yl)-3,4-dihydro-5-oxa-1,2a-diazaacenaphthylen-7-yl)methanone (106),
(R)-(3-aminopiperidin-1-yl)(2-(6-methoxy-1-(2-methoxyethyl)-1H-indol-2-yl)-3,4-dihydro-5-oxa-1,2a-diazaacenaphthylen-7-yl)methanone (107),
(R)-(3-aminopiperidin-1-yl)(2-(1-(2-hydroxyethyl)-1H-indol-2-yl)-3,4-dihydro-5-oxa-1,2a-diazaacenaphthylen-7-yl)methanone (108),
(R)-(3-aminopiperidin-1-yl)(2-(1-(cyclopropylmethyl)-6-methoxy-1H-indol-2-yl)-3,4-dihydro-5-oxa-1,2a-diazaacenaphthylen-7-yl)methanone (109),
(R)-(3-aminopiperidin-1-yl)(2-(1-((3-fluoropyridin-4-yl)methyl)-1H-indol-2-yl)-3,4-dihydro-5-oxa-1,2a-diazaacenaphthylen-7-yl)methanone (110),
(R)-(3-aminopiperidin-1-yl)(2-(1-(pyrazin-2-ylmethyl)-1H-indol-2-yl)-3,4-dihydro-5-oxa-1,2a-diazaacenaphthylen-7-yl)methanone (111),
(R)-(3-aminopiperidin-1-yl)(2-(1-((3-fluoropyridin-2-yl)methyl)-1H-indol-2-yl)-3,4-dihydro-5-oxa-1,2a-diazaacenaphthylen-7-yl)methanone (112),
(R)-(3-aminopiperidin-1-yl)(2-(1-(pyrimidin-2-ylmethyl)-1H-indol-2-yl)-3,4-dihydro-5-oxa-1,2a-diazaacenaphthylen-7-yl)methanone (113),
(R)-(3-aminopiperidin-1-yl)(2-(1-(cyclopropylmethyl)-6-fluoro-1H-indol-2-yl)-3,4-dihydro-5-oxa-1,2a-diazaacenaphthylen-7-yl)methanone (114),
(R)-(3-aminopiperidin-1-yl)(2-(1-(pyrimidin-5-ylmethyl)-1H-indol-2-yl)-3,4-dihydro-5-oxa-1,2a-diazaacenaphthylen-7-yl)methanone (115),
(R)-(3-aminopiperidin-1-yl)(2-(1-(pyridazin-3-ylmethyl)-1H-indol-2-yl)-3,4-dihydro-5-oxa-1,2a-diazaacenaphthylen-7-yl)methanone (116),
(R)-(3-aminopiperidin-1-yl)(2-(1-isobutyl-1H-indol-2-yl)-3,4-dihydro-5-oxa-1,2a-diazaacenaphthylen-7-yl)methanone (117),
(R)-(3-aminopiperidin-1-yl)(2-(1-(cyclopropylmethyl)-5,6-difluoro-1H-indol-2-yl)-3,4-dihydro-5-oxa-1,2a-diazaacenaphthylen-7-yl)methanone (118),
(R)-(3-aminopiperidin-1-yl)(2-(1-((3-fluoropyridin-2-yl)methyl)-6-methoxy-1H-indol-2-yl)-3,4-dihydro-5-oxa-1,2a-diazaacenaphthylen-7-yl)methanone (119),
(R)-(3-aminopiperidin-1-yl)(2-(7-chloro-1-((3-fluoropyridin-2-yl)methyl)-1H-indol-2-yl)-3,4-dihydro-5-oxa-1,2a-diazaacenaphthylen-7-yl)methanone (120),
(R)-(3-aminopiperidin-1-yl)(2-(6-fluoro-1-((3-fluoropyridin-2-yl)methyl)-1H-indol-2-yl)-3,4-dihydro-5-oxa-1,2a-diazaacenaphthylen-7-yl)methanone (121),
(R)-(3-aminopiperidin-1-yl)(2-(7-chloro-1-(2-methoxyethyl)-1H-indol-2-yl)-3,4-dihydro-5-oxa-1,2a-diazaacenaphthylen-7-yl)methanone (122),
(R)-(3-aminopiperidin-1-yl)(2-(1-(4-(hydroxymethyl)benzyl)-1H-indol-2-yl)-3,4-dihydro-5-oxa-1,2a-diazaacenaphthylen-7-yl)methanone trifluoroacetic acid salt (123),
(R,E)-N-(1-(2-(1-(cyclopropylmethyl)-1H-indol-2-yl)-3,4-dihydro-5-oxa-1,2a-diazaacenaphthylene-7-carbonyl)piperidin-3-yl)-4-(dimethylamino)but-2-enamide trifluoroacetic acid salt (124),
(R)-(3-aminopiperidin-1-yl)(2-(7-chloro-1-(4-methoxybenzyl)-1H-indol-2-yl)-3,4-dihydro-5-oxa-1,2a-diazaacenaphthylen-7-yl)methanone (125),
(R)-(3-aminopiperidin-1-yl)(2-(1-((tetrahydro-2H-pyran-4-yl)methyl)-1H-indol-2-yl)-3,4-dihydro-5-oxa-1,2a-diazaacenaphthylen-7-yl)methanone (126),
(R)-(3-aminopiperidin-1-yl)(2-(1-(cyclobutylmethyl)-1H-indol-2-yl)-3,4-dihydro-5-oxa-1,2a-diazaacenaphthylen-7-yl)methanone (127),
(R)-2-(2-(7-(3-aminopiperidine-1-carbonyl)-3,4-dihydro-5-ox1,2adiazaacenaphthylen-2-yl)-1H-indol-1-yl)acetic acid (128),
(R)-(3-aminopiperidin-1-yl)(2-(1-(piperidin-4-ylmethyl)-1H-indol-2-yl)-3,4-dihydro-5-oxa-1,2a-diazaacenaphthylen-7-yl)methanone (129),
(R)-(3-aminopiperidin-1-yl)(2-(1-(oxetan-3-ylmethyl)-1H-indol-2-yl)-3,4-dihydro-5-oxa-1,2a-diazaacenaphthylen-7-yl)methanone (130),
(R)-(3-aminopiperidin-1-yl)(2-(1-((1-methylpiperidin-4-yl)methyl)-1H-indol-2-yl)-3,4-dihydro-5-oxa-1,2a-diazaacenaphthylen-7-yl)methanone (131),
(R)-(3-aminopiperidin-1-yl)(2-(5-fluoro-1-(4-methoxybenzyl)-1H-indol-2-yl)-3,4-dihydro-5-oxa-1,2a-diazaacenaphthylen-7-yl)methanone (132),
(R)-(3-aminopiperidin-1-yl)(2-(1-(2,2-difluoroethyl)-1H-indol-2-yl)-3,4-dihydro-5-oxa-1,2a-diazaacenaphthylen-7-yl)methanone (133),
(R)-(3-aminopiperidin-1-yl)(2-(5-fluoro-1-(2-methoxyethyl)-1H-indol-2-yl)-3,4-dihydro-5-oxa-1,2a-diazaacenaphthylen-7-yl)methanone (134),
(R)-(3-aminopiperidin-1-yl)(2-(6-fluoro-1-(4-fluorobenzyl)-1H-indol-2-yl)-3,4-dihydro-5-oxa-1,2a-diazaacenaphthylen-7-yl)methanone (135),
(R)-(3-aminopiperidin-1-yl)(2-(6-fluoro-1-(4-methoxybenzyl)-1H-indol-2-yl)-3,4-dihydro-5-oxa-1,2a-diazaacenaphthylen-7-yl)methanone (136),
(R)-(3-aminopiperidin-1-yl)(2-(1-(4-fluorobenzyl)-6-methoxy-1H-indol-2-yl)-3,4-dihydro-5-oxa-1,2a-diazaacenaphthylen-7-yl)methanone (137),
3-aminopiperidin-1-yl)(2-(6-fluoro-1-(2-methoxyethyl)-1H-indol-2-yl)-3,4-dihydro-5-oxa-1,2a-diazaacenaphthylen-7-yl)methanone (138),
(R)-(3-aminopiperidin-1-yl)(2-(7-chloro-1-(cyclobutylmethyl)-1H-indol-2-yl)-3,4-dihydro-5-oxa-1,2a-diazaacenaphthylen-7-yl)methanone (139),
(R)-(3-aminopiperidin-1-yl)(2-(5,6-difluoro-1-(2-methoxyethyl)-1H-indol-2-yl)-3,4-dihydro-5-oxa-1,2a-diazaacenaphthylen-7-yl)methanone (140),
(R)-(3-aminopiperidin-1-yl)(2-(7-chloro-1-isobutyl-1H-indol-2-yl)-3,4-dihydro-5-oxa-1,2a-diazaacenaphthylen-7-yl)methanone (141),
(R)-(3-aminopiperidin-1-yl)(2-(7-chloro-1-(2,2-difluoroethyl)-1H-indol-2-yl)-3,4-dihydro-5-oxa-1,2a-diazaacenaphthylen-7-yl)methanone (142),
(R)-(3-aminopiperidin-1-yl)(2-(7-chloro-1-(cyclopropylmethyl)-1H-indol-2-yl)-3,4-dihydro-5-oxa-1,2a-diazaacenaphthylen-7-yl)methanone (143),
(R,E)-(3-aminopiperidin-1-yl)(2-(1-(cyclopropylmethyl)-5-styryl-1H-indol-2-yl)-3,4-dihydro-5-oxa-1,2a-diazaacenaphthylen-7-yl)methanone (144),
(R)-(3-aminopiperidin-1-yl)(2-(1-((4-methylthiazol-2-yl)methyl)-1H-indol-2-yl)-3,4-dihydro-5-oxa-1,2a-diazaacenaphthylen-7-yl)methanone (145),
(R)-(3-aminopiperidin-1-yl)(2-(1-(cyclopropylmethyl)-5-methoxy-1H-indol-2-yl)-3,4-dihydro-5-oxa-1,2a-diazaacenaphthylen-7-yl)methanone (146),
(R)-(3-aminopiperidin-1-yl)(2-(1-(cyclopropylmethyl)-6-(hydroxymethyl)-1H-indol-2-yl)-3,4-dihydro-5-oxa-1,2a-diazaacenaphthylen-7-yl) methanone (147),
(R)-(3-aminopiperidin-1-yl)(2-(1-(cyclopropylmethyl)-1H-indol-2-yl)-3,3-dimethyl-3,4-dihydro-5-oxa-1,2a-diazaacenaphthylen-7-yl)methanone (148),
(R)-(3-aminopiperidin-1-yl)(2-(1-(cyclopropylmethyl)-1H-indol-2-yl)-5,6-dihydro-4H-imidazo[1,5,4-de]quinoxalin-8-yl)methanone (149),
(R)-(3-aminopiperidin-1-yl)(2-(1-(cyclopropylmethyl)-5-fluoro-1H-indol-2-yl)-5,6-dihydro-4H-imidazo[1,5,4-de]quinoxalin-8-yl)methanone (150),
(R)-(3-aminopiperidin-1-yl)(2-(1-(cyclopropylmethyl)-6-fluoro-1H-indol-2-yl)-5,6-dihydro-4H-imidazo[1,5,4-de]quinoxalin-8-yl)methanone (151),
(R)-(3-aminopiperidin-1-yl)(2-(1-(pyridin-3-ylmethyl)-1H-indol-2-yl)-5,6-dihydro-4H-imidazo[1,5,4-de]quinoxalin-8-yl)methanone (152),
(R)-(3-aminopiperidin-1-yl)(2-(5-bromo-1-(cyclopropylmethyl)-1H-indol-2-yl)-5,6-dihydro-4H-imidazo[1,5,4-de]quinoxalin-8-yl)methanone (153),
(R)-(3-aminopiperidin-1-yl)(2-(1-(cyclopropylmethyl)-5-(pyridin-3-yl)-1H-indol-2-yl)-5,6-dihydro-4H-imidazo[1,5,4-de]quinoxalin-8-yl)methanone (154),
(R)-(3-aminopiperidin-1-yl)(2-(1-(pyridin-2-ylmethyl)-1H-indol-2-yl)-5,6-dihydro-4H-imidazo[1,5,4-de]quinoxalin-8-yl)methanone (155),
(R)-(3-aminopiperidin-1-yl)(2-(1-(2-fluorobenzyl)-1H-indol-2-yl)-5,6-dihydro-4H-imidazo[1,5,4-de]quinoxalin-8-yl)methanone (156),
(R)-(3-aminopiperidin-1-yl)(2-(1-(pyridin-4-ylmethyl)-1H-indol-2-yl)-5,6-dihydro-4H-imidazo[1,5,4-de]quinoxalin-8-yl)methanone (157),
(R)-(3-aminopiperidin-1-yl)(2-(1-(cyclopropylmethyl)-1H-indol-2-yl)-6-methyl-5,6-dihydro-4H-imidazo[1,5,4-de]quinoxalin-8-yl)methanone (158),
(R)-(3-aminopiperidin-1-yl)(2-(1-(cyclopropylmethyl)-6-methoxy-1H-indol-2-yl)-5,6-dihydro-4H-imidazo[1,5,4-de]quinoxalin-8-yl)methanone (159),
(R)-(3-aminopiperidin-1-yl)(2-(1-(cyclopropylmethyl)-6-methoxy-1H-indol-2-yl)-6-methyl-5,6-dihydro-4H-imidazo[1,5,4-de]quinoxalin-8-yl)methanone (160),
(R)-(3-aminopiperidin-1-yl)(2-(1-(cyclopropylmethyl)-5-fluoro-1H-indol-2-yl)-6-methyl-5,6-dihydro-4H-imidazo[1,5,4-de]quinoxalin-8-yl)methanone (161),
(R)-(3-aminopiperidin-1-yl)(2-(1-(cyclopropylmethyl)-1H-indol-2-yl)-6-(methylsulfonyl)-5,6-dihydro-4H-imidazo[1,5,4-de]quinoxalin-8-yl)methanone (162),
(R)-(3-aminopiperidin-1-yl)(2-(1-(cyclopropylmethyl)-5,6-difluoro-1H-indol-2-yl)-5,6-dihydro-4H-imidazo[1,5,4-de]quinoxalin-8-yl)methanone (163),
(R)-(3-aminopiperidin-1-yl)(6-cyclopropyl-2-(1-(cyclopropylmethyl)-1H-indol-2-yl)-5,6-dihydro-4H-imidazo[1,5,4-de]quinoxalin-8-yl)methanone (164),
(R)-(3-aminopiperidin-1-yl)(2-(1-(cyclopropylmethyl)-1H-indol-2-yl)-6-(phenethylsulfonyl)-5,6-dihydro-4H-imidazo[1,5,4-de]quinoxalin-8-yl)methanone (165),
(R)-(3-aminopiperidin-1-yl)(2-(1-(cyclopropylmethyl)-4-fluoro-1H-indol-2-yl)-5,6-dihydro-4H-imidazo[1,5,4-de]quinoxalin-8-yl)methanone (166),
(R)-(3-aminopiperidin-1-yl)(6-((4-chlorophenyl)sulfonyl)-2-(1-(cyclopropylmethyl)-1H-indol-2-yl)-5,6-dihydro-4H-imidazo[1,5,4-de]quinoxalin-8-yl)methanone (167),
(R)-(3-aminopiperidin-1-yl)(2-(1-(cyclopropylmethyl)-1H-indol-2-yl)-6-(cyclopropylsulfonyl)-5,6-dihydro-4H-imidazo[1,5,4-de]quinoxalin-8-yl)methanone (168),
(R)-(3-aminopiperidin-1-yl)(2-(1-(cyclopropylmethyl)-1H-indol-2-yl)-6-((2-ethoxyethyl)sulfonyl)-5,6-dihydro-4H-imidazo[1,5,4-de]quinoxalin-8-yl)methanone (169),
(R)-(3-aminopiperidin-1-yl)(2-(1-(cyclopropylmethyl)-7-methyl-1H-indol-2-yl)-5,6-dihydro-4H-imidazo[1,5,4-de]quinoxalin-8-yl)methanone (170),
(R)-1-(8-(3-aminopiperidine-1-carbonyl)-2-(1-(cyclopropylmethyl)-1H-indol-2-yl)-4,5-dihydro-6H-imidazo[1,5,4-de]quinoxalin-6-yl)ethan-1-one (171),
(R)-(3-aminopiperidin-1-yl)(2-(1-(cyclopropylmethyl)-1H-indol-2-yl)-5,6-dihydro-4H-imidazo[1,5,4-de]quinoxalin-8-yl)methanethione (172),
(R)-(3-aminopiperidin-1-yl)(2-(7-chloro-1-(cyclopropylmethyl)-6-fluoro-1H-indol-2-yl)-3,4-dihydro-5-oxa-1,2a-diazaacenaphthylen-7-yl)methanone (173), and
(R)-(3-aminopiperidin-1-yl)(2-(7-chloro-1-(pyrimidin-5-ylmethyl)-1H-indol-2-yl)-3,4-dihydro-5-oxa-1,2a-diazaacenaphthylen-7-yl)methanone (174).

4. The method as claimed in claim 1, wherein a disease or disorder or condition is selected from cancer, cancer metastasis, immune disorder, immunodeficiency disorder, inflammatory disorder, and transplant rejection.

5. The method as claimed in claim 4, wherein the disease or disorder or condition is cancer.

6. The method as claimed in claim 1, wherein the disease or disorder or condition is selected from systemic inflammatory response syndrome, decreasing neutrophil extra cellular traps (NETs) release, acute lung injury (ALI), acute respiratory distress syndrome (ARDS), COVID-19 (corona virus disease-2019) related lung infection, multi-organ failure or multi-organ dysfunction syndrome from ARDS, anti neutrophil cytoplasmic antibody (ANCA) associated vasculitis (AAV), hidradenitis suppurativa (HS), sepsis, infections, or cytokine storms induced by drugs or any agent, ischemic or haemorrhagic stroke, ischemic or drug-induced haemorrhagic transformation in the brain, haemorrhagic encephalopathy, traumatic brain injury, anoxic brain injury, chronic kidney disease, diabetes, deep vein thrombosis, systemic microthrombosis, atherosclerotic thrombosis, thromboembolism, systemic lupus erythematosus (SLE), rheumatoid arthritis, chronic obstructive pulmonary disease (COPD), cystic fibrosis, pulmonary disease, inflammatory bowel disease (IBS), crohn's disease, ulcerative colitis, indeterminate colitis, or alzheimer's disease.

7. The method as claimed in claim 5, wherein the cancer is selected from breast cancer, prostate cancer, pancreatic cancer, gastric cancer, lung cancer, colon cancer, rectal cancer, esophagus cancer, duodenal cancer, tongue cancer, pharyngeal cancer, brain tumor, neurinoma, clear cell carcinoma, non-small cell lung cancer, small cell lung cancer, liver cancer, kidney cancer, bile duct cancer, uterine body cancer, cervical cancer, ovarian cancer, urinary bladder, skin cancer, hemangioma, malignant lymphoma, malignant melanoma, thyroid cancer, bone tumor, vascular fibroma, glioblastoma, sarcoma, neuroendocrine tumors, retinoblastoma, penile cancer, pediatric solid cancer, renal cell carcinoma, lymphoma, myeloma, leukemia, acute myelogenous leukemia (AML), chronic myelogenous leukemia (CML), chronic neutrophilic leukemia, chronic eosinophilic leukemia, chronic lymphocytic leukemia (CLL), acute lymphoblastic leukemia (ALL), hairy cell leukemia, cutaneous T-cell lymphoma (CTCL), multiple myeloma (MM), myeloproliferative neoplasms (MPN), polycythemia vera (PV), essential thrombocythemia, essential thrombocytosis (ET), myelofibrosis (MF), chronic myelogenous leukemia (CML), chronic neutrophilic leukemia (CNL), or chronic eosinophilic leukemia (CEL).

8. The method as claimed in claim 4, wherein the condition is cancer metastasis, and the cancer metastasis is selected from the group consisting of liver cancer metastasis, lung cancer metastasis, and omentum cancer metastasis.

9. The method as claimed in claim 8, wherein the cancer metastasis is selected from liver cancer metastasis originating from colorectal cancer and pancreatic cancer, lung cancer metastasis originating from breast cancer, and omentum cancer metastasis originating from ovarian cancer.

10. The method as claimed in claim 1, wherein the composition is a pharmaceutical composition comprising the compound together with a pharmaceutically acceptable carrier, optionally in combination with one or more other pharmaceutical compositions.

11. The method as claimed in claim 10, wherein the composition is in the form selected from a group consisting of a tablet, capsule, powder, syrup, solution, aerosol, and suspension.

12. The method as claimed in claim 1, wherein the method comprising administering a combination of the compounds or the composition of claim 1, with other clinically relevant cytotoxic agents or non-cytotoxic agents to a subject in need thereof.

13. The method as claimed in claim 1, wherein the method comprising administering a combination of the compounds or the composition of claim 1, with other clinically relevant immune modulator agents or anti-inflammatory agents to a subject in need of thereof.

14-23. (canceled)
